(19) United States
(12) Patent Application Publication
(10) Pub. No.: US 2009/0143368 A1
(43) Pub. Date: Jun. 4, 2009

(54) Use of Cinnamoyl Compound

(76) Inventors: Hiroaki Shiraki, Rockville, MD (US); Kiyoshi Higashi, Osaka-shi (JP); Yoshitaka Tomigahara, Toyonaka-shi (JP); Junya Takahashi, Kawabe-gun (JP)

Correspondence Address: WENDEROTH, LIND & PONACK, L.L.P., 1030 15th Street, N.W.,, Suite 400 East, Washington, DC, 20005-1503, US

(21) Appl. No.: 11/885,379

(22) PCT Filed: Mar. 2, 2006
(86) PCT No.: PCT/JP2006/304539

§ 371 Date: Oct. 1, 2007

(30) Foreign Application Priority Data
Mar. 2, 2005 (JP) 2005-057364

(51) Int. Cl. A61K 31/5415; C07D 471/04; A61K 31/4375; A61K 31/5377; C07D 413/14; C07D 279/16

(52) U.S. Cl. 514/226.5; 546/123; 514/300; 514/232.5; 544/80; 544/49

(57) ABSTRACT

A composition for inhibiting the extracellular matrix gene transcription comprising a cinnamoyl compound represented by the formula (I):

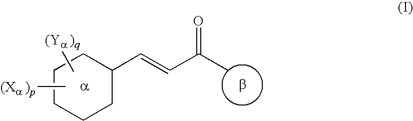

and an inert carrier, or the like.

### TECHNICAL FIELD

The present invention relates to use of a cinnamoyl compound and the like.

### BACKGROUND ART

In diseases and disorders such as hepatic cirrhosis, chronic pancreatitis, scirrhous gastric cancer, interstitial pulmonary disease, asthma, chronic obstructive pulmonary diseases, glomerular nephritis, lupus nephritis, tubulointerstitial nephritis, IgA nephritis, renal sclerosis, diabetic nephropathy, hereditary renal disease, myocardial fibrosis, heart failure, restenosis after PTCA, atherosclerosis, marrow fibrosis, rheumatoid arthritis, hyperplasia scar after inflammation, postoperative scars or burn scars, atopic dermatitis, hypertrophic scar, hysteromyoma, prostate hypertrophy, scleroderma, Alzheimer disease, sclerotic peritonitis, diabetic retinopathy, and type I diabetes, excessive accumulation of an extracellular matrix, a representative of which is collagen or fibronectin, causes fibrosis and sclerosis of tissues, resulting in decreased functions, cicatrization and the like in the organs or tissues. Such excessive accumulation of an extracellular matrix is induced by increased production of the extracellular matrix due to a breakdown of balance between biosynthesis and degradation of the extracellular matrix. In fact, it has been observed that expression of an extracellular matrix gene such as a collagen gene (in particular, a Type I collagen gene, a Type III collagen gene, or a Type IV collagen gene), a fibronectin gene, a laminin gene, a proteoglycan gene or the like has been increased in a fibrotic tissue [J. Invest. Dermatol., 94, 365, (1990); Proc. Natl. Acad. Sci. USA, 88, 6642, (1991); J. Am. Soc. Nephrol., 15, 2637, (2004); Cardiovasc. Pathol., 13, 119, (2004); Clin. Nephrol., 44, 211, (1995); J. Hepatol., 29, 263, (1998))].

It has been also observed that the amount of TGF-β, which is a cytokine, has been increased in a fibrotic tissue [e.g. J. Invest. Dermatol., 94, 365, (1990) and Proc. Natl. Acad. Sci. USA, 88, 6642, (1991)]. It has been suggested that TGF-β has increased expression of an extracellular matrix gene and been involved in increased production of an extracellular matrix protein and, consequently, fibrosis of a tissue [e.g. J. Invest. Dermatol., 94, 365, (1990); and Lab. Invest., 63, 171, (1990)]. It has been also shown that by administering an anti-TGF-β antibody or a soluble anti-TGF-β receptor to a model animal of tissue fibrosis, improvement of tissue fibrosis has been achieved and thereby the tissue function has been also improved [e.g. Diabetes, 45, 522-530, (1996), Proc. Natl. Acad. Sci. USA, 96, 12719-12724, (1999); and Proc. Natl. Acad. Sci. USA, 97, 8015-8020, (2000)]. It has been also known that by administering a compound which suppressively acts on intracellular signal transduction via TGF-β, improvement in fibrosis of a tissue has been achieved and thereby the tissue function has been also improved [e.g. Autoimmunity, 35, 277-282, (2002); J. Hepatol., 37, 331-339, (2002); and Life Sci., 71, 1559-1606, (2002)].

On the other hand, it is believed that a cause of heart failure such as left ventricular diastolic failure or renal failure such as diabetic nephropathy or renal sclerosis is cardiac fibrosis under a hypertensive condition.

Thus, there is a need for development and provision of a drug which improves fibrosis of a tissue by decreasing expression of an extracellular matrix gene in the tissue to reduce accumulation of the extracellular matrix (i.e. an extracellular matrix accumulation-suppressing agent, a fibrosing disease-treating agent, or a heart failure-treating agent).

### DISCLOSURE OF INVENTION

The present invention relates to compounds represented by the following formulas (I) to (VI), (I′), (II′) and (V′) having the ability to suppress transcription of an extracellular matrix gene.

That is, the present invention provides:

1. A composition for suppressing transcription of an extracellular matrix gene which comprises an inert carrier and a cinnamoyl compound represented by the formula (I):

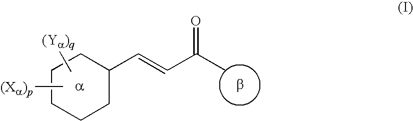

wherein,

I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;

(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ″ and R_{d} are as defined above), a C2-C10 alkenyl group or a C2-C10 alkynyl group;

(2) the Y₀ group: a M_{b0}-R_{d}— group, wherein M_{b0} represents a M_{c0}- group

[wherein M_{co} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by

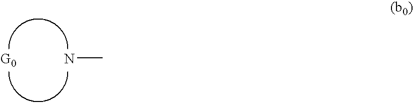

(wherein G₀ forms an optionally substituted, saturated or unsaturated, nonaromatic 5 to 14-membered hydrocarbon ring or heterocyclic ring), a (c₀)- group represented by

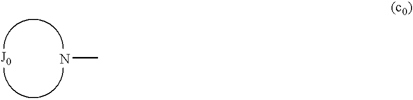

(wherein, J₀ forms a 5 to 7-membered aromatic ring optionally containing a nitrogen atom), a (d₀)- group represented by

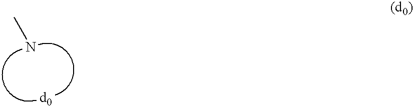

[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group), a sulfinyl group, or a sulfonyl group], or a (e₀)- group represented by

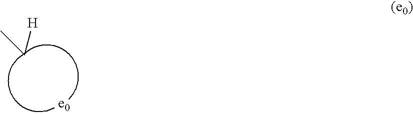

{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group); and R_{d}′ is the same as or different from R_{d} and has the same meaning as R_{d} has],
a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;

(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and

II. β represents

a group represented by formula (I-1):

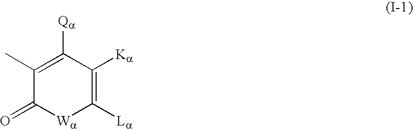

wherein,
(1) Q_{α} represents an optionally substituted hydroxyl group, or an optionally substituted amino group,
(2) W_{α} represents an oxygen atom or a —NT_{α}— group (wherein T_{α} represents a hydrogen atom, or a substituent on the nitrogen atom),
(3) K_{α} and L_{α} form a —V_{α}═V_{α}′V_{α}′″═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (I-2):

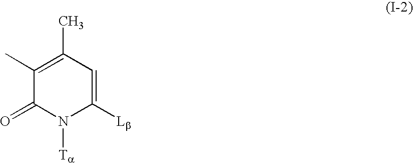

wherein T_{α} is as defined above, and L_{β} represents a hydroxyl group or a methyl group;
a group represented by formula (I-3):

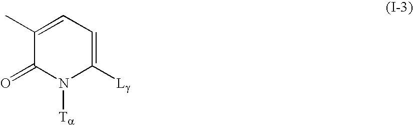

wherein T_{α} is as defined above, and L_{γ} represents a C1-C10 alkyl group;
a group represented by formula (I-4):

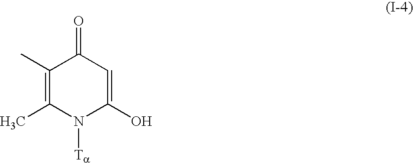

wherein T_{α} is as defined above;
a group represented by formula (I-5):

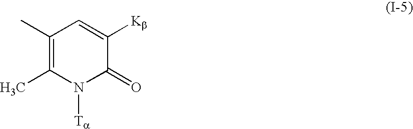

wherein T_{α} is as defined above, and K_{β} represents a cyano group or a UOCO— group (wherein U represents a hydrogen atom or a C1-C10 alkyl group);
a group represented by formula (I-6):

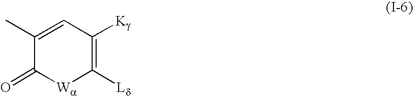

wherein W_{α} is as defined above, and K_{γ} and L_{δ} form an optionally substituted C3-C10 alkylene group or an optionally substituted C4-C10 alkenylene group;
a group represented by formula (I-7):

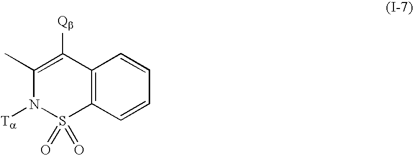

wherein T_{α} is as defined above, and Q_{β} represents an optionally substituted hydroxyl group; or
a group represented by formula (I-8):

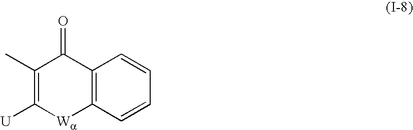

wherein U and W_{α} are as defined above;

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

2. A cinnamoyl compound represented by the formula (II):

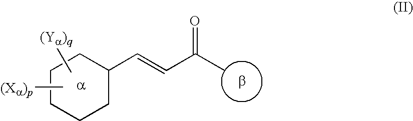

wherein,

I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})_{p, X}_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;

(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d} group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;

(2) the Y₀ group: a M_{b0}-R_{d}— group, wherein M_{b0} represents a M_{c0}- group

[wherein M_{c0} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by

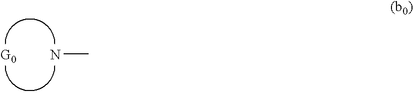

(wherein G₀ forms an optionally substituted, saturated or unsaturated, nonaromatic 5 to 14-membered hydrocarbon ring or heterocyclic ring), a (c₀)- group represented by

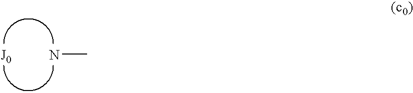

(wherein, J₀ forms a 5 to 7-membered aromatic ring optionally containing a nitrogen atom), a (d₀)- group represented by

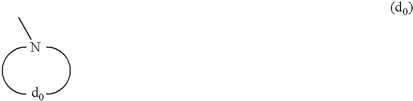

[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group}, a sulfinyl group, or a sulfonyl group], or a (e₀)- group represented by

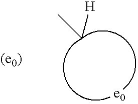

{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}; and R_{d}′ is the same as or different from R_{d} and has the same meaning as R_{d} has],
a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}OCO—NRₑ— group (wherein M_{c0} and Rₑ are as defined above), a M_{c0}RₑN—CO— group (wherein M_{c0} and Rₑ are as defined above), a M_{c0}RₑN—CO NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;

(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and

II. β represents

a group represented by formula (II-1):

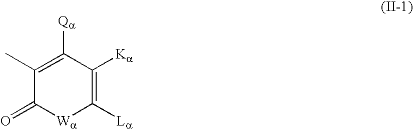

wherein,
(1) Q_{α} represents an optionally substituted hydroxyl group, or an optionally substituted amino group,
(2) W_{α} represents an oxygen atom or a —NT_{α}— group (wherein T_{α} represents a hydrogen atom, or a substituent on the nitrogen atom),
(3) K_{α} and L_{α} form a —V_{α}═V_{α}″−V_{α}═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″, and V_{α}″ represents a —N═ group);
a group represented by formula (II-2):

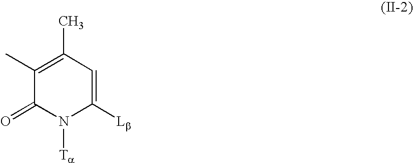

wherein T_{α} is as defined above, and L_{β} represents a hydroxyl group or a methyl group;
a group represented by formula (II-3):

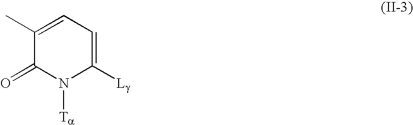

wherein T_{α} is as defined above, and L_{γ} represents a C1-C10 alkyl group;
a group represented by formula (II-4):

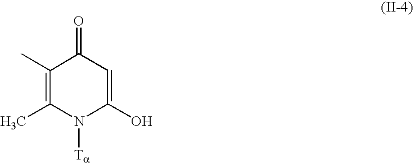

wherein T_{α} is as defined above;
a group represented by formula (II-5):

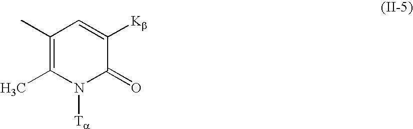

wherein T_{α} is as defined above, and K_{β} represents a cyano group or a UOCO— group (wherein U represents a hydrogen atom or a C1-C10 alkyl group);
a group represented by formula (II-6):

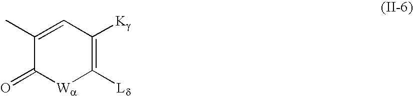

wherein W_{α} is as defined above, and K_{γ} and L_{δ} form an optionally substituted C3-C10 alkylene group or an optionally substituted C4-C10 alkenylene group;
a group represented by formula (II-7):

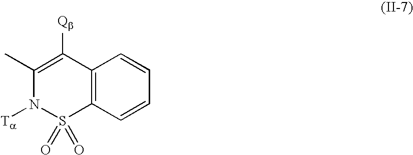

wherein T_{α} is as defined above, and Q_{β} represents an optionally substituted hydroxyl group; or
a group represented by formula (II-8):

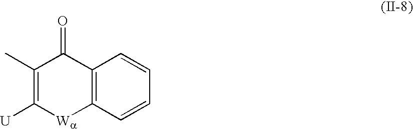

wherein U and W_{α} are as defined above;

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

3. A cinnamoyl compound represented by the formula (III):

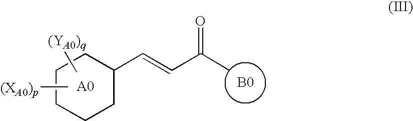

wherein

I. A0 represents a benzene ring or pyridine ring;

II. in (X_{A0})ₚ, X_{A0} is a substituent on a carbon atom and represents a group included in any group of the following A₀ group to the N₀ group, p represents 0, 1, 2, 3, 4 or 5, and when p is not lees than 2, X_{A0}s are the same or different;

(1) the A₀ group:

a D₁-R₄— group, wherein D₁ represents a (R₁—(O)ₖ—)A₁N—(O)_{k′}— group [wherein R₁ represents a hydrogen atom, or a C1-C10 alkyl group, or a C2-C10 alkyl group substituted with a halogen atom or a R₂—B₁-group (wherein R₂ represents a C1-C10 alkyl group, a C3-C10 alkenyl group or a C3-C10 alkynyl group, and B₁ represents an oxy group, a thio group, a sulfinyl group or a sulfonyl group), or a C3-C10 alkenyl group, or a C3-C10 alkynyl group, k represents 0 or 1, A₁ represents a R₃—(CHR₀)ₘ—(B₂—B₃)_{m′}— group {wherein R₃ represents a hydrogen atom, or a C1-C10 alkyl group optionally substituted with a halogen atom or a R₂—B₁— group (wherein R₂ and B₁ are as defined above), or a C2-C10 alkenyl group, or a C2-C10 alkynyl group, Rₒ represents a hydrogen atom, a C1-C10 alkyl group or a C2-C10 haloalkyl group, m represents 0 or 1, B₂ represents a single bond, an oxy group, a thio group or a —N((O)R₁′)- group (wherein R₁′ is the same as or different from R₁, and has the same meaning as R₁ has, and n represents 0 or 1), B₃ represents a carbonyl group, a thiocarbonyl group or a sulfonyl group, m′ represents 0 or 1, and B₃ is not a sulfonyl group when m is 0 and R₃ is a hydrogen atom}, and k′ represents 0 or 1], and R₄ represents a C1-C10 alkylene group, provided that a R₀′R₀″N—R₄— group (wherein R₀′ and R₀″ are the same as or different from Rₒ and have the same meaning as R₀ has, and R₄ is as defined above) is excluded,

a D₂-R₄— group, wherein D₂ represents a cyano group, a R₁R₁′NC(═N—(O)ₙ-A₁)- group (wherein R₁, R₁′, n and A₁ are as defined above), an A₁N═C(—OR₂)— group (wherein A₁ and R₂ are as defined above) or a NH₂—CS— group, and R₄ is as defined above,

a D₃-R₄— group, wherein D₃ represents a nitro group or a R₁OSO₂— group (wherein R₁ is as defined above), and R₄ is as defined above, and

a R₁OSO₂— group, wherein R₁ is as defined above;

(2) the B₀ group: an (a₀)- group represented by

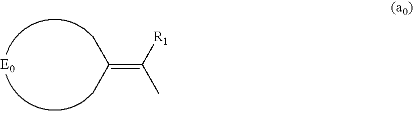

wherein E₀ forms an optionally substituted, saturated or unsaturated, aromatic or nonaromatic 5 to 14-membered hydrocarbon ring or heterocyclic ring, and R₁ is as defined above;

(3) the Co group: a C2-C10 alkenyl group substituted with a halogen atom, a R₂—B₁— group (wherein R₂ and B₁ are as defined above), a D₄-R₄— group [wherein D₄ represents a hydroxyl group or an A₁-O— group (wherein A₁ is as defined above), and R₄ is as defined above], a D₅- group [wherein D₅ represents a O═C(R₃)— group (wherein R₃ is as defined above), an A₁-(O)ₙ—N═C(R₃)— group (wherein A₁, n and R₃ are as defined above), a R₁—B₀—CO—R₄—(O)ₙ—N═C(R₃)— group {wherein R₁, R₄, n and R₃ are as defined above, and Bo represents an oxy group, a thio group or a —N((O)ₘR₁′)— group (wherein R₁′ and m are as defined above)}, a D₂-R₄—(O)ₙ—N═C(R₃)— group (wherein D₂, R₄, n and R₃ are as defined above) or a R₁A₁N—N═C(R₃)— group (wherein R₁, A₁ and R₃ are as defined above)], a R₁A₁N—O—R₄— group (wherein R₁, A₁ and R₄ are as defined above), a R₁(A₁-(O)ₙ—)N— group (wherein R₁, A₁ and n are as defined above), a D₂- group (wherein D₂ is as defined above) or a D₃- group (wherein D₃ is as defined above);

(4) the D₀ group: a C2-C10 alkynyl group substituted with a (b₀)-R₄— group (in (b₀)

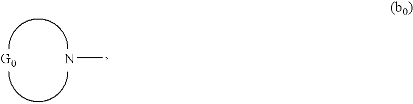

G₀ forms an optionally substituted, saturated or unsaturated, nonaromatic 5 to 14-membered hydrocarbon ring or heterocyclic ring), a (c₀)-R₄— group (in (c₀)

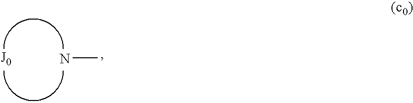

J₀ forms an aromatic 5 to 7-membered ring optionally containing a nitrogen atom and R₄ is as defined above), a halogen atom, a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above) or a D₃-R₄— group (wherein D₃ and R₄ are as defined above);

(5) the E₀ group: an A₂-CO—R₅— group, provided that R₅ is not a vinylene group when A₂ is a hydroxyl group [wherein A₂ represents

(i) an A₃-B₄— group

wherein A₃ represents a hydrogen atom, or a C1-C10 alkyl group, or a C2-C10 haloalkyl group, or a C2-C10 alkenyl group optionally substituted with a halogen atom, or a C3-C10 alkynyl group optionally substituted with a halogen atom, or a RₐO—(R₄)ₘ— group (wherein Rₐ₀ represents an optionally substituted 5 to 7-membered aryl group or heteroaryl group, and R₄ and m are as defined above), or a C1-C10 alkyl group substituted with a (b₀)-R₄— group (wherein (b₀) and R₄ are as defined above), a (c₀)-R₄— group (wherein (c₀) and R₄ are as defined above), a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above), a D₃-R₄— group (wherein D₃ and R₄ are as defined above) or an A₄-SO₂—R₄— group (wherein A₄ represents a (b₀)- group (wherein (b₀) is as defined above), a (c₀)- group (wherein (c₀) is as defined above) or a R₁R₁′N— group (wherein R₁ and R₁′ are as defined above), and R₄ is as defined above}, and
B₄ represents an oxy group, a thio group or a —N((O)ₘR₁)— group (wherein R₁ and m are as defined above), provided that A₃ is not a hydrogen atom when B₄ is a thio group;

(ii) a R₁—B₄—CO—R₄—B₄′— group (wherein R₁, B₄ and R₄ are as defined above, B₄′ is the same as or different from B₄ and has the same meaning as B₄ has, provided that R₂ is not a hydrogen atom when B₄ is a thio group) or a D₂-R₄—B₄— group (wherein D₂, R₄ and B₄ are as defined above);

(iii) a R₂—SO₂—NR₁— group (wherein R₂ is as defined above, provided that a hydrogen atom is excluded, and R₁ is as defined above);

(iv) a (b₀)- group, wherein (b₀) is as defined above;

(v) a (c₀)- group, wherein (c₀) is as defined above; or

(vi) a R₁A₁N—NR₁′— group, wherein R₁, A₁ and R₁′ are as defined above; and

R₅ represents a C2-C10 alkenylene group optionally substituted with a halogen atom or a C2-C10 alkynylene group];

(6) the F₀ group: an A₅-B₅—R₆— group, wherein

A₅ represents a C2-C10 alkyl group substituted with a D₄- group (wherein D₄ is as defined above), a D₁- group (wherein D₁ is as defined above), a D₃- group (wherein D₃ is as defined above) or an A₄-SO₂— group (wherein A₄ is as defined above), or a C1-C10 alkyl group substituted with a R₂—B₁— group (wherein R₂ and B₁ are as defined above), a D₂- group (wherein D₂ is as defined above), a D₅- group (wherein D₅ is as defined above) or an A₂-CO— group (wherein A₂ is as defined above), and

B₅ represents a B₁— group (wherein B₁ is as defined above) or a —NA₁- group (wherein A₁ is as defined above) and R₆ represent a single bond or a C1-C10 alkylene group;

(7) the G₀ group: an A₆-B₅—R₆— group wherein

A₆ represents an (a₀)-R₄— group (wherein (a₀) and R₄ are as defined above), or a C2-C10 alkenyl group, or a C2-C10 alkynyl group, or a C2-C10 alkenyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as defined above), or a C2-C10 alkynyl group substituted with a halogen atom, a R₂—B₁— group (wherein R₂ and B₁ are as defined above), a D₅- group (wherein D₅ is as defined above), D₂- group (wherein D₂ is as defined above) or an A₂-CO— group (wherein A₂ is as defined above), or a C3-C10 alkenyl group substituted with a (b₀)- group (wherein (b₀) is as defined above), a (c₀)- group (wherein (c₀) is as defined above), a D₄- group (wherein D₄ is as defined above), a D₁- group (wherein D₁ is as defined above) or a D₃- group (wherein D₃ is as defined above), or a C3-C10 alkynyl group substituted with a D₄- group (wherein D₄ is as defined above), a D₁- group (wherein D₁ is as defined above) or a D₃- group (wherein D₃ is as defined above), and

B₅ and R₆ are as defined above;

(8) the H₀ group:

a D₂-N(—(O)ₙ-A₁)-R₆— group (wherein D₂, n, A₁ and R₆ are as defined above),

a D₂- group (wherein D₂ is as defined above, provided that a cyano group is excluded),

a R₁(R₁′(O)ₙ)N—CR₁″═N—R₆— group (wherein R₁, R₁′, n and R₆ are as defined above, R₁″ is the same as or different from R₁ and has the same meaning as R₁ has),

a R₁— (O)ₙ—N═CR₁′—NR₂—R₆— group (wherein R₁, n, R₁′, R₂ and R₆ are as defined above),

a R₂—B₃—NR₁—CO—NR₁′—R₆— group (wherein R₂, B₃, R₁, R₁′ and R₆ are as defined above),

a D₂-CO—NR₁—R₆— group (wherein D₂, R₁ and R₆ are as defined above), and

an A₂-COCO—NR₁—R₆— group (wherein A₂, R₁ and R₆ are as defined above);

(9) the I₀ group:

an A₇-B₆—N((O)ₙR₁)—R₆— group [wherein A₇ represents a C2-C10 alkenyl group optionally substituted with a halogen atom, or a C2-C10 alkynyl group, or a C3-C10 haloalkynyl group, or a R₂—B₁—R₄— group (wherein R₂, B₁ and R₄ are as defined above), or a D₄-R₄— group (wherein D₄ and R₄ are as defined above), or a D₅-R₄— group (wherein D₅ and R₄ are as defined above), or a D₁-R₄— group (wherein D₁ and R₄ are as defined above), or a (b₀)-R₄— group (wherein (b₀) and R₄ are as defined above), or a (c₀)-R₄— group (wherein (c₀) and R₄ are as defined above), or a D₂-R₄— group (wherein D₂ and R₄ are as defined above), or a D₃-R₄— group (wherein D₃ and R₄ are as defined above), or an A₄-SO₂—R₄— group (wherein A₄ and R₄ are as defined above), or an A₂-CO—R₄— group (wherein A₂ and R₄ are as defined above), B₆ represents a carbonyl group or a thiocarbonyl group, and n, R₁ and R₆ are as defined above],

an A₈-CS—N((O)ₙR₁)—R₆— group [wherein A₈ represents a hydrogen atom or a C1-C10 alkyl group optionally substituted with a halogen atom, and n, R₁ and R₄ are as defined above],

an A₇′-B₂′—B₃—N((O)ₙR₁′)—R₆— group [wherein A₇′ represents a C3-C10 alkenyl group optionally substituted with a halogen atom, or a C3-C10 alkynyl group optionally substituted with a halogen atom, or a R₂—B₁—R₄′— group (wherein R₂ and B₁ are as defined above, and R₄′ represents a C2-C10 alkylene group), or a D₄-R₄′— group (wherein D₄ and R₄′ are as defined above), or a D₁-R₄′— group (wherein D₁ and R₄′ are as defined above), or a (b₀)-R₄′— group (wherein (b₀) and R₄′ are as defined above), or a (c₀)-R₄′— group (wherein (c₀) and R₄′ are as defined above), or a D₂-R₄— group (wherein D₂ and R₄ are as defined above), or a D₃-R₄′— group (wherein D₃ and R₄′ are as defined above), or an A₂-CO—R₄— group (wherein A₂ and R₄ are as defined above), B₂′ represents an oxy group, a thio group or a —N((O)_{n′}R₁′)— group (wherein n′ is the same as or different from n and has the same meaning as n has, and R₁′ is as defined above), and B₃, n, R₁ and R₆ are as defined above], an A₈′-B₂′-CS—N((O)ₙR₁)—R₆— group [wherein A₈′ represents a C1-C10 alkyl group or a C2-C10 haloalkyl group, B₂′ is as defined above, and n, R₁ and R₆ are as defined above],

an A₈′-S—B₃′—N((O)ₙR₁)—R₆— group [wherein A₈′, n, R₁ and R₆ are as defined above, and B₃′ represents a carbonyl group or a sulfonyl group], and

an A₇″-SO₂—N((O)ₙR₁)—R₆— group [wherein A₇″ represents a C2-C10 alkenyl group, or a C3-C10 alkenyl group substituted with a halogen atom, or a C3-C10 alkynyl group optionally substituted with a halogen atom, or a R₂—B₁—R₄′— group (wherein R₂, B₁ and R₄′ are as defined above), or a D₄-R₄′— group (wherein D₄ and R₄′ are as defined above), or a D₅-R₄— group (wherein D₅ and R₄ are as defined above), or a D₁-R₄′— group (wherein D₁ and R₄′ are as defined above), or a (b₀)-R₄′— group (wherein (b₀) and R₄′ are as defined above), or a (c₀)-R₄′— group (wherein (c₀) and R₄′ are as defined above), or a D₂-R₄— group (wherein D₂ and R₄ are as defined above), or a NO₂—R₄— group (wherein R₄ is as defined above), or an A₂-CO—R₄— group (wherein A₂ and R₄ are as defined above), and n, R₁ and R₆ are as defined above];

(10) the J₀ group:

an A₇-CO— group (wherein A₇ is as defined above),

an A₉-CS— group (wherein A₉ represents A₇ or A₈),

an A₉′ (O)ₘN═C(A₉)- group (wherein A₉′ represents A₇′ or A₈′, and m and A₉ are as defined above),

a D₂-CO— group (wherein D₂ is as defined above),

an A₂-COCO— group (wherein A₂ is as defined above),

an A₉-CO—B₁′ —R₆— group (wherein A₉ and R₆ are as defined above, and B₁′ represents an oxy group or a thio group, provided that A₉ is not A₈ when B₁′ is an oxy group),

an A₉-CS—B₁′—R₆— group (wherein A₉, B₁′ and R₆ are as defined above),

an A₇″-SO₂—B₁′—R₆— group (wherein A₇″, B₁′ and R₆ are as defined above),

an A₈-SO₂—B₁′—R₆— group (wherein A₈, B₁′ and R₆ are as defined above, provided that A₈ is not a hydrogen atom),

an A₉′-B₂′—B₃—B₁′ —R₆— group (wherein A₉′, B₂′, B₃, B₁′ and R₆ are as defined above), and

a C2-C10 alkenyl group substituted with a (b₀)- group (wherein (b₀) is as defined above) or a (c₀)- group (wherein (c₀) is as defined above);

(11) the K₀ group: an A₁₀-N((O)ₙR)—CO—R₆— group, wherein A₁₀ represents a hydrogen atom (provided that n is not 0), an A₇″-SO₂— group (wherein A₇″ is as defined above),

an A₈-SO₂— group (wherein A₈ is as defined above, provided that A₈ is not a hydrogen atom), an A₉′O— group (wherein A₉′ is as defined above, (provided that n is not 1), an A₉′— group (wherein A₉′ is as defined above, provided that A₈′ is excluded when n is 0), a R₂OCH₂— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above;

(12) the L₀ group:

an A₁₀′-N((O)ₙR₁)—SO₂—R₆— group [wherein A₁₀′ represents a hydrogen atom (provided that n is not 1), an A₉′O— group (wherein A₉′ is as defined above, provided that n is not 0), an A₉′- group (wherein A₉′ is as defined above, provided that A₈′ is excluded when n is 0), a R₂—CO— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above],

an A₉″R₁N—SO₂—N((O)ₙR₁′)—R₆— group [wherein A₉″ represents a hydrogen atom or an A₉′- group (wherein A₉′ is as defined above), and R₁, n, R₁′ and R₆ are as defined above] and

a (b₀)-SO₂—N((O)ₙR₁′)—R₆— group [wherein (b₀), n, R₁′ and R₆ are as defined above];

(13) the M₀ group:

a R₁(R₂S)C═N—R₆— group (wherein R₁, R₂ and R₆ are as defined above),

a R₂B(R₂′B′)C═N—R₆— group (wherein R₂ and R₆ are as defined above, R₂′ is the same as or different from R₂ and has the same meaning as R₂ has, and B and B′ are the same or different and represent an oxy group or a thio group),

a R₁R₁′N—(R₂S)C═N—R₆— group (wherein R₁, R₁′, R₂ and R₆ are as defined above),

a R₁N═C(SR₂)—NR₂′—R₆— group (wherein R₁, R₂, R₂′ and R₆ are as defined above), and

a R₁(R₁′O)N—R₆— group (wherein R₁, R₁′ and R₆ are as defined above);

(14) the N₀ group: a A₁₁-P(═O) (OR₁′)—R₄— group, wherein A₁₁ represents a R₁— group (wherein R₁ is as defined above), a R₁O—R₆— group (wherein R₁ and R₆ are as defined above) or a R₁OCO—CHR₀— group (wherein R₁ and R₀ are as defined above), and R₁′ and R₄ are as defined above;

III. in (Y_{A0})_{q}, Y_{A0} is a substituent on a carbon atom and represents a group included in the following X₀ group and Y₀ group, q represents 0, 1, 2, 3, 4 or 5, the sum of p (wherein p is as defined above) and q is not more than 5, and when q is not less than 2, Y_{A0}s are the same or different, and when q is not less than 2, adjacent two same or different Y_{A0}s may together form a group included in the Z₀ group to be fused to the A0 ring;

(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;

(2) the Y₀ group: a M_{b0}-R_{d}— group, wherein M_{b0} represents a M_{c0}- group [wherein M_{co} represents a M_{do}-R_{d}′— group (wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by

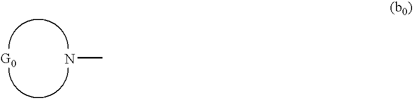

(wherein (b₀) is as defined above), a (c₀)- group represented by

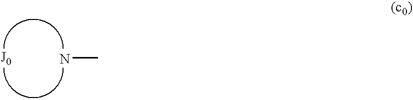

(wherein, (c₀) is as defined above), a (d₀)- group represented by

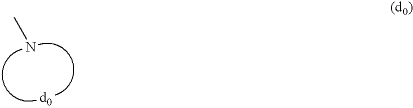

{wherein d₀ forms a 5 to 12-membered hydrocarbon ring which is substituted with a carbonyl group or a thiocarbonyl group and further which may be optionally substituted with an oxy group, a thio group, a —NR₁— group (wherein R₁ is as defined above), a sulfinyl group or a sulfonyl group}, or a (e₀)- group represented by

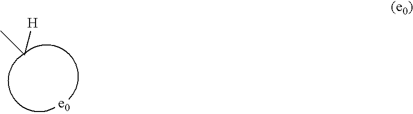

{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}, and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above), or a M_{c0}RₑN—SO₂— group (wherein M_{c0} and Rₑ are as defined above), and R_{d} is as defined above;

(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the A0 ring; and

IV. B0 is a group represented by formula (III-1):

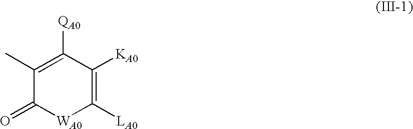

[wherein,
(1) Q_{A0} represents a hydroxyl group, a (b₀)- group (wherein (b₀) is as defined above), an A₉-B₆—B_{c}— group [wherein A₉ and B₆ are as defined above, and B_{c} represents an oxy group or a N—((O)ₘR₁) group (wherein m and R₁ are as defined above), provided that B_{c} is not a sulfonyl group when A₉ is a hydrogen atom], an A₇″-SO₂—B_{c}— group (wherein A₇″ and B_{c} are as defined above), an A₈-SO₂—B_{c}— group (wherein A₈ and B_{c} are as defined above, provided that A₈ is not a hydrogen atom), a R₁R₁′N—SO₂—B_{c}— group (wherein R₁, R₁′ and B_{c} are as defined above), a (b₀)-SO₂—B_{c}— group (wherein (b₀) and B_{c} are as defined above), an A₉′-B_{c}— group (wherein A₉′ and B_{c} are as defined above), a D₅-R₄—B_{c}— group (wherein D₅, R₄ and B_{c} are as defined above), a M_{c0}-B₃—B_{c}— group (wherein M_{c0}, B₃ and B_{c} are as defined above) or a M_{c0}-B_{c}— group (wherein M_{c0} and B_{c} are as defined above),
(2) W_{A0} represents an oxygen atom or a —NT_{A0}- group wherein T_{A0} represents a hydrogen atom, an A₉′- group (wherein A₉′ is as defined above), a D₅-R₄— group (wherein D₅ and R₄ are as defined above) or a M_{c0}- group (wherein M_{c0} is as defined above),
(3) K_{A0} and L_{A0} form a —V_{A0}═V_{A0}″, —V_{A0}″═V_{A0}′″— group {wherein V_{A0}, V_{A0}′, V_{A0}″ and V_{A0}′″ are the same or different, and represent a methine group optionally substituted with a Mₐ- group (wherein Mₐ is as defined above), or a —N═ group, and at least one of V_{A0}, V_{A0}′, V_{A}0″ and V_{A0}′″ represents a —N═ group},

a group represented by formula (III-2):

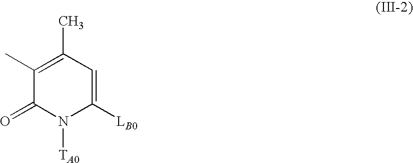

[wherein T_{A0} is as defined above, and L_{B0} represents a hydroxyl group or a methyl group],

a group represented by formula (III-3):

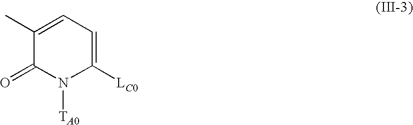

[wherein T_{A0} is as defined above, and L_{C0} represents a C1-C10 alkyl group],

a group represented by formula (III-4):

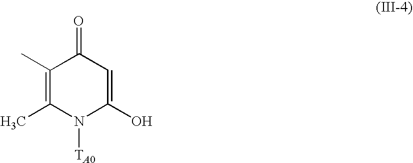

[wherein T_{A0} is as defined above],

a group represented by formula (III-5):

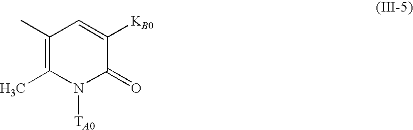

[wherein T_{A0} is as defined above, and K_{B0} represents a cyano group or a UOCO— group (wherein U represents a hydrogen atom or a C1-C10 alkyl group)],

a group represented by formula (III-6):

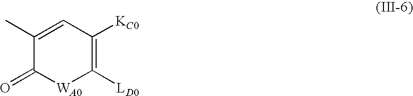

[wherein W_{A0} is as defined above, and K_{c0} and L_{D0} form a C3-C10 alkylene group, or a C4-C10 alkenylyne optionally substituted with 1 or more same or different Mₐ- groups (wherein Mₐ is as defined above)],

a group represented by formula (III-7):

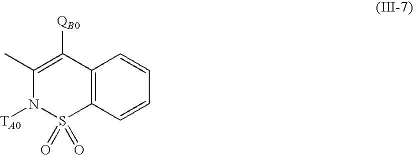

[wherein T_{A0} is as defined above, and Q_{B0} represents a hydroxyl group, a A₉-B₆—O— group (wherein A₉ and B₆ are as defined above), an A₇″-SO₂—O— group (wherein A₇″ is as defined above), an A₈-SO₂—O— group, (wherein A₈ is as defined above, provided that A₈ is not a hydrogen), a R₁R₁′N—SO₂—O— group (wherein R₁ and R₁′ are as defined above), a (b₀)-SO₂—O— group (wherein (b₀) is as defined above), an A₉′—O— group (wherein A₉′ is as defined above), a D₅-R₄—O— group (wherein D₅ and R₄ are as defined above), a M_{c0}-B₃—O— group (wherein M_{c0} and B₃ are as defined above) or a M_{c0}-O—O— group (wherein M_{c0} is as defined above)], or

a group represented by formula (III-8):

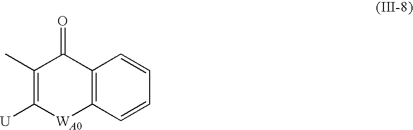

[wherein U and W_{A0} are as defined above]; and

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

4. A cinnamoyl compound represented by the formula (IV):

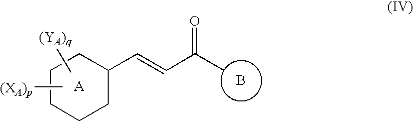

wherein,

I. A represents a benzene ring or a pyridine ring;

II. in (X_{A})ₚ, X_{A} is a substituent on a carbon atom, and represents a group included in any group of the following A group to N group, p represents 0, 1, 2, 3, 4 or 5 and, when p is not less than 2, X_{A}s are the same or different;

(1) the A group:

a D₁-R₄— group, wherein D₁ represents a (R₁—(O)ₖ-(A₁N—(O)ₖ′— group [wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), or a C3-C10 alkenyl group, or a C3-C10 alkynyl group, k represents 0 or 1, A₁ represents a R₃—(CHR₀)ₘ—(B₂—B₃)ₘ— group {wherein R₃ represents a hydrogen atom, or a C1-C10 alkyl group optionally substituted with a halogen atom or a R₂—B₁— group (wherein R₂ and B₁ are as defined above), or a C2-C10 alkenyl group, or a C2-C10 alkynyl group, Rₒ represents a hydrogen atom, a C1-C10 alkyl group or a C2-C10 haloalkyl group, m represents 0 or 1, B₂ represents a single bond, an oxy group, a thio group or a —N((O)ₙR₁′)- group (wherein R₁′ is the same as or different from R₁ and has the same meaning as R₁ has, and n represents 0 or 1), B₃ represents a carbonyl group, a thiocarbonyl group or a sulfonyl group, m′ represents 0 or 1, and B₃ is not a sulfonyl group when m is 0 and R₃ is a hydrogen atom}, and k′ represents 0 or 1], and R₄ represents a C1-C10 alkylene group, provided that a R₀′R₀″N—R₄— group (wherein R₀′ and R₀″ are the same as or different from Rₒ and have the same meaning as Rₒ has, and R₄ is as defined above) is excluded,

a D₂-R₄— group, wherein D₂ represents a cyano group, a R₁R₁′NC(═N—(O)ₙ-A₁)- group (wherein R₁, R₁′, n and A₁ are as defined above), an A₁N═C(—OR₂)— group (wherein A₁ and R₂ are as defined above) or a NH₂—CS— group, and R₄ is as defined above,

a D₃-R₄— group, wherein D₃ represents a nitro group or a R₁OSO₂— group (wherein R₁ is as defined above), and R₄ is as defined above, and

a R₁OSO₂— group, wherein R₁ is as defined above;

(2) the B group: an (a)-group represented by

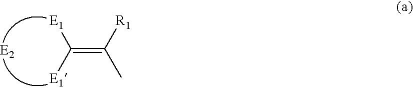

wherein E₁ and E₁′ represent a methylene group optionally substituted with a C1-C10 alkyl group or a C1-C10 alkoxy group, or a carbonyl group, provided that E₁ and E₁′ are not a carbonyl group at the same time, E₂ represents a C2-C10 alkylene group optionally substituted with an oxy group, a thio group, a sulfinyl group, a sulfonyl group or a —NR₁′— group (wherein R₁′ is as defined above), or a C3-C10 alkenylene group optionally substituted with an oxy group, a thio group, a sulfinyl group, a sulfonyl group or a —NR₁′— group (wherein R₁′ is as defined above), and R₁ is as defined above;

(3) the C group: a C2-C10 alkenyl group substituted with a halogen atom, a R₂—B₁— group (wherein R₂ and B₁ are as defined above), a D₄-R₄— group [wherein D₄ represents a hydroxyl group or an A₁-O— group (wherein A₁ is as defined above), and R₄ is as defined above], a D₅- group [wherein D₅ represents an O═C(R₃)— group (wherein R₃ is as defined above), an A₁-(O)ₙ—N═C(R₃)— group (wherein A₁, n and R₃ are as defined above), a R₁—B₀—CO—R₄—(O)ₙ—N═C(R₃)— group {wherein R₁, R₄, n and R₃ are as defined above, and B₀ represents an oxy group, a thio group or a —N((O)ₘR₁′)— group (wherein R₁′ and m are as defined above)), a D₂-R₄—(O)ₙ—N═C(R₃)— group (wherein D₂, R₄, n and R₃ are as defined above) or a R₁A₁N—N═C(R₃)— group (wherein R₁, A₁ and R₃ are as defined above)], a R₁A₁N—O—R₄— group (wherein R₁, A₁ and R₄ are as defined above), a R₁ (A₁-(O)ₙ—)N— group (wherein R₁, A₁ and n are as defined above), a D₂- group (wherein D₂ is as defined above) or a D₃- group (wherein D₃ is as defined above);

(4) the D group: a C2-C10 alkynyl group substituted with a (b) —R₄— group [wherein in (b)

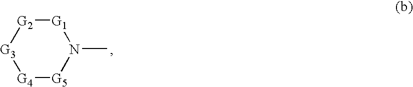

G₁, G₂, G₄ and G₅ represent a methylene group which is connected with the adjacent atom via a single bond and which may be optionally substituted with a methyl group, or a methine group which is connected with the adjacent atom via a double bond and which may be optionally substituted with a methyl group, and G₃ represents a single bond, or a double bond, or a C1-C10 alkylene group optionally substituted with a methyl group, an oxy group, a thio group, a sulfinyl group, a sulfonyl group or a —NR₁— group (wherein R₁ is as defined above), or a C2-C10 alkenylene group optionally substituted with a methyl group, an oxy group, a thio group, a sulfinyl group, a sulfonyl group or a —NR₁— group (wherein R₁ is as defined above); and R₄ is as defined above], a (c) —R₄— group [wherein, in (c)

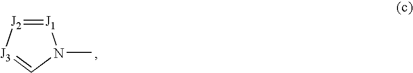

J₁, J₂ and J₃ are the same or different, and represent a methine group optionally substituted with a methyl group, or a nitrogen atom; and R₄ is as defined above), a halogen atom, a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above) or a D₃-R₄— group (wherein D₃ and R₄ are as defined above);

(5) the E group: an A₂-CO—R₅— group, provided that R₅ is not a vinylene group when A₂ is a hydroxyl group, [wherein A₂ represents

(i) an A₃-B₄— group

wherein A₃ represents a hydrogen atom, or a C1-C10 alkyl group, or a C2-C10 haloalkyl group, or a C2-C10 alkenyl group optionally substituted with a halogen atom, or a C3-C10 alkynyl group optionally substituted with a halogen atom, or Rₐ—(R₄)ₘ— group (wherein Rₐ represents a phenyl group, a pyridyl group, a furyl group or a thienyl group, which may be optionally substituted with a halogen atom, a C1-C10 alkyl group, a C1-C10 alkoxy group or a nitro group, and R₄ and m are as defined above), or a C1-C10 alkyl group substituted with a (b) —R₄— group (wherein (b) and R₄ are as defined above), a (c) —R₄— group (wherein (c) and R₄ are as defined above), a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above), a D₃-R₄— group (wherein D₃ and R₄ are as defined above) or an A₄-SO₂—R₄— group {wherein A₄ represents a (b)- group (wherein (b) is as defined above), a (c)- group (wherein (c) is as defined above) or a R₁R₁′N— group (wherein R₁ and R₁′ are as defined above), and R₄ is as defined above}, and B₄ represents an oxy group, a thio group or a —N((O)ₘR₁)— group (wherein R₁ and m are as defined above), provided that A₃ is not a hydrogen atom when B₄ is a thio group;

(ii) a R₁—B₄—CO—R₄—B₄′— group, wherein R₁, B₄ and R₄ are as defined above, B₄′ is the same as or different from B₄ and has the same meaning as B₄ has, provided that R₂ is not a hydrogen atom when B₄ is a thio group, or

a D₂-R₄—B₄— group, wherein D₂, R₄ and B₄ are as defined above;

(iii) a R₂—SO₂—NR₁— group, wherein R₂ is as defined above, provided that a hydrogen atom is excluded, and R₁ is as defined above;

(iv) a (b)- group, wherein (b) is as defined above,

(v) a (c)- group, wherein (c) is as defined above, or

(vi) a R₁A₁N—NR₁′— group, wherein R₁, A₁ and R₁′ are as defined above; and

R₅ represents a C2-C10 alkenylene group optionally substituted with a halogen atom or a C2-C10 alkynylene group];

(6) the F group: an A₅-B₅—R₆— group

[wherein A₅ represents a C2-C10 alkyl group substituted with a D₄- group (wherein D₄ is as defined above), a D₁- group (wherein D₁ is as defined above), a D₃- group (wherein D₃ is as defined above) or an A₄-SO₂— group (wherein A₄ is as defined above), or a C1-C10 alkyl group substituted with a R₂—B₁— group (wherein R₂ and B₁ are as defined above), a D₂- group (wherein D₂ is as defined above), a D₅- group (wherein D₅ is as defined above) or an A₂-CO— group (wherein A₂ is as defined above),

B₅ represents a B₁— group (wherein B₁ is as defined above) or a —NA₁- group (wherein A₁ is as defined above), and

R₆ represents a single bond or a C1-C10 alkylene group];

(7) the G group: an A₆-B₅—R₆— group

[wherein A₆ represents an (a) —R₄— group (wherein (a) and R₄ are as defined above), or a C2-C10 alkenyl group, or a C2-C10 alkynyl group, or a C2-C10 alkenyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as define above), or a C2-C10 alkynyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as defined above), or a C3-C10 alkenyl group substituted with a (b)- group (wherein (b) is as defined above), a (c)- group (wherein (c) is as defined above), a D₄- group (wherein D₄ is as defined above), a D₁- group (wherein D₁ is as defined above) or a D₃- group (wherein D₃ is as defined above), or a C3-C10 alkynyl group substituted with a D₄- group (wherein D₄ is as defined above), a D₁- group (wherein D₁ is as defined above) or a D₃- group (wherein D₃ is as defined above), and B₅ and R₆ are as defined above];

(8) the H group:

a D₂-N(—(O)ₙ-A₁)-R₆— group, wherein D₂, n, A₁ and R₆ are as defined above,

a D₂- group, wherein D₂ is as defined above, provided that a cyano group is excluded,

a R₁ (R₁′ (O)ₙ)N—CR₁″═N—R₆— group, wherein R₁, R₁′, n and R₆ are as defined above, R₁″ is the same as or different from R₁ and has the same meaning as R₁ has,

a R₁—(O)ₙ—N═CR₁′-NR₂—R₆— group, wherein R₁, n, R₁′, R₂ and R₆ are as defined above,

a R₂—B₃—NR₁—CO—NR₁′-R₆— group, wherein R₂, B₃, R₁, R₁′ and R₆ are as defined above,

a D₂-CO—NR₁—R₆— group, wherein D₂, R₁ and R₆ are as defined above, or

an A₂-COCO—NR₁—R₆— group, wherein A₂, R₁ and R₆ are as defined above;

(9) the I group:

an A₇-B₆—N((O)ₙR₁′)—R₆— group [wherein A₇ represents a C2-C10 alkenyl group optionally substituted with a halogen atom, or a C2-C10 alkynyl group, or a C3-C10 haloalkynyl group, or a R₂—B₁—R₄— group (wherein R₂, B₁ and R₄ are as defined above), or a D₄-R₄— group (wherein D₄ and R₄ are as defined above), or a D₅-R₄— group (wherein D₅ and R₄ are as defined above), or a D₁-R₄— group (wherein D₁ and R₄ are as defined above), or a (b) —R₄— group (wherein (b) and R₄ are as defined above), or a (c) —R₄— group (wherein (c) and R₄ are as defined above), or a D₂-R₄— group (wherein D₂ and R₄ are as defined above), or a D₃-R₄— group (wherein D₃ and R₄ are as defined above), or an A₄-SO₂—R₄— group (wherein A₄ and R₄ are as defined above), or an A₂-CO—R₄— group (wherein A₂ and R₄ are as defined above), B₆ represents a carbonyl group or a thiocarbonyl group, and n, R₁ and R₆ are as defined above],

an A₈-CS—N((O)ₙR₁)—R₆— group [wherein A₈ represents a hydrogen atom or a C1-C10 alkyl group optionally substituted with a halogen atom, and n, R₁ and R₆ are as defined above],

an A₇′-B₂′—B₃—N((O)ₙR₁)—R₆— group [wherein A₇′ represents a C3-C10 alkenyl group optionally substituted with a halogen atom, or a C3-C10 alkynyl group optionally substituted with a halogen atom, or a R₂—B₁—R₄′— group (wherein R₂ and B₁ are as defined above, and R₄′ represents a C2-C10 alkylene group), or a D₄-R₄′— group (wherein D₄ and R₄′ are as defined above), or a D₁-R₄′— group (wherein D₁ and R₄′ are as defined above), or a (b) —R₄′— group (wherein (b) and R₄′ are as defined above), or a (c) —R₄′— group (wherein (c) and R₄′ are as defined above), or a D₂-R₄— group (wherein D₂ and R₄ are as defined above), or a D₃-R₄′— group (wherein D₃ and R₄′ are as defined above), or an A₂-CO—R₄— group (wherein A₂ and R₄ are as defined above), B₂′ represents an oxy group, a thio group or a —N((O)_{n′}R₁′)— group (wherein n′ is the same as or different from n and has the same meaning as n has, and R₁′ is as defined above), and B₃, n, R₁ and R₆ are as defined above],

an A₈′-B₂′-CS—N((O)ₙR₁′)—R₄— group [wherein A₈′ represents a C1-C10 alkyl group or a C2-C10 haloalkyl group, B₂′ is as defined above, and n, R₁ and R₆ are as defined above],

an A₈′-S—B₃′—N((O)ₙR)—R₆— group [wherein A₈′, n, R₁ and R₆ are as defined above, and B₃′ represents a carbonyl group or a sulfonyl group] or

an A₇″-SO₂—N((O)ₙR₁)—R₆— group [wherein A₇″ represents a C2-C10 alkenyl group, or a C3-C10 alkenyl group substituted with a halogen atom, or a C3-C10 alkynyl group optionally substituted with a halogen atom, or a R₂—B₁—R₄′— group (wherein R₂, B₁ and R₄′ are as defined above), or a D₄-R₄′— group (wherein D₄ and R₄′ are as defined above), or a D₅-R₄— group (wherein D₅ and R₄ are as defined above), or a D₁-R₄′— group (wherein D₁ and R₄′ are as defined above), or a (b) —R₄′— group (wherein (b) and R₄′ are as defined above), or a (c) —R₄′— group (wherein (c) and R₄′ are as defined above), or a D₂-R₄— group (wherein D₂ and R₄ are as defined above), or a NO₂—R₄— group (wherein R₄ is as defined above), or an A₂-CO—R₄— group (wherein A₂ and R₄ are as defined above), and n, R₁ and R₄ are as defined above];

(10) the J group:

an A₇-CO— group (wherein A₇ is as defined above),

an A₉-CS— group (wherein A₉ represents A₇ or A₈),

an A₉′ (O)ₙN═C(A₉)- group (wherein A₉′ represents A₇′ or A₈′, and m and A₉ are as defined above),

a D₂-CO— group (wherein D₂ is as defined above),

an A₂-COCO— group (wherein A₂ is as defined above),

an A₉-CO—B₁′ —R₆— group (wherein A₉ and R₆ are as defined above, and B₁′ represents an oxy group or a thio group, provided that A₉ is not A₈ when B₁₁ is an oxy group),

an A₉-CS—B₁′—R₆— group (wherein A₉, B₁′ and R₆ are as defined above),

an A₇″-SO₂—B₁′—R₆— group (wherein A₇″, B₁′ and R₆ are as defined above),

an A₈-SO₂—B₁′—R₆— group (wherein A₈, B₁′ and R₆ are as defined above, provided that A₈ is not a hydrogen atom),

an A₉′-B₂′—B₃—B₁′—R₆— group (wherein A₉′, B₂′, B₃, B₁′ and R₆ are as defined above), and

a C2-C10 alkenyl group substituted with a (b)- group (wherein (b) is as defined above) or a (c)- group (wherein (c) is as defined above);

(11) the K group: an A₁₀-N((O)ₙR₁)—CO—R₆— group [wherein A₁₀ represents a hydrogen atom (provided that n is not 0), an A₇″-SO₂— group (wherein A₇″ is as defined above), an A₈-SO₂— group (wherein A₈ is as defined above, provided that A₈ is not a hydrogen atom), an A₉′O— group (wherein A₉′ is as defined above, provided that n is not 1), an A₉′- group (wherein A₉′ is as defined above, provided that A₈′ is excluded when n is 0), a R₂OCH₂— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above];

(12) the L group:

an A₁₀′-N((O)ₙR₁)—SO₂—R₆— group (wherein A₁₀′ represents a hydrogen atom (provided that n is not 0), an A₉′O— group (wherein A₉′ is as defined above, provided that n is not 1), an A₉′- group (wherein A₉′ is as defined above, provided that A₈′ is excluded when n is 0), a R₂—CO— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above],

an A₉″R₁N—SO₂—N((O)ₙR₁′)—R₆— group [wherein A₉″ represents a hydrogen atom or an A₉′- group (wherein A₉′ is as defined above), and R₁, n, R₁′ and R₆ are as defined above], and

a (b)-SO₂—N((O)ₙR₁′)—R₆— group [wherein (b), n, R₁′ and R₆ are as defined above];

(13) the M group:

a R₁(R₂S)C═N—R₆— group (wherein R₁, R₂ and R₆ are as defined above),

a R₂B(R₂′B′)C═N—R₆— group (wherein R₂ and R₆ are as defined above, R₂′ is the same as or different from R₂ and has the same meaning as R₂ has, and B and B′ are the same or different and represent an oxy group or a thio group),

a R₁R₁′N—(R₂S)C═N—R₆— group (wherein R₁, R₁′, R₂ and R₆ are as defined above),

a R₁N═C(SR₂)—NR₂′—R₆— group (wherein R₁, R₂, R₂′ and R₆ are as defined above), and

a R₁(R₁′O)N—R₆— group (wherein R₁, R₁′ and R₆ are as defined above);

(14) the N group: an A₁₁-P(═O) (OR₁′)—R₄— group [wherein A₁₁ represents a R₁— group (wherein R₁ is as defined above), a R₁O—R₆— group (wherein R₁ and R₆ are as defined above) or a R₁OCO—CHR₀— group (wherein R₁ and R₀ are as defined above), and R₁′ and R₄ are as defined above];

III. in (Y_{A})_{q}, Y_{A} is a substituent on a carbon atom and represents a group included in the following X group or Y group, q represents 0, 1, 2, 3, 4 or 5, and the sum of p (wherein p is as defined above) and q is not more than 5, and when q is not less than 2, Y_{A}s are the same or different, and when q is not less than 2, adjacent two same or different Y_{A}s may together form a group included in the Z group to be fused to the A ring;

(1) the X group: a Mₐ-group, wherein Mₐ represents a R_{b}— group (wherein R_{b} represents a C1-C10 alkyl group optionally substituted with a halogen atom), a halogen atom, a nitro group, a cyano group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;

(2) the Y group: a M_{b}-R_{d}— group, wherein M_{b} represents a M_{c}-group [wherein M_{c} represents a M_{d}-R_{d}′— group {wherein M_{d} represents a phenyl group optionally substituted with a Mₐ- group (wherein Mₐ is as defined above), a pyridyl group optionally substituted with a Mₐ- group (wherein Mₐ is as defined above), a naphthyl group optionally substituted with a Mₐ- group (wherein Mₐ is as defined above), a (b)- group (wherein (b) is as defined above), a (c)- group (wherein (c) is as defined above), a (d)- group

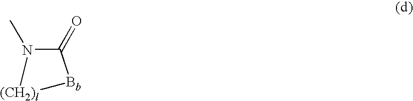

(wherein l is 2, 3 or 4, B_{b} represents an oxy group or a thio group) or an (e)- group

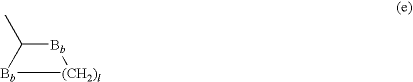

(wherein l and B_{b} are as defined above)}, and R_{d}′ is the same as or different from R_{d} and has the same meaning as R_{d} has], a M_{c}-Bₐ— group (wherein M_{c} and Bₐ are as defined above), a M_{c}-CO— group (wherein M_{c} is as defined above), a M_{c}-CO—O— group (wherein M_{c} is as defined above), a M_{c}O—CO— group (wherein M_{c} is as defined above), a M_{c}RₑN— group (wherein M_{c} and Rₑ are as defined above), a M_{c}-CO—NRₑ— group (wherein M_{c} and Rₑ are as defined above), a M_{c}O—CO—NRₑ— group (wherein M_{c} and Rₑ are as defined above), a M_{c}RₑN—CO— group (wherein M_{c} and Rₑ are as defined above), a M_{c}RₑN—CO—NRₑ′— group (wherein M_{c}, Rₑ and Rₑ′ are as defined above), a M_{c}RₑN—C(═NRₑ′)—NRₑ″— group (wherein M_{c}, Rₑ, Rₑ′ and Rₑ″ are as defined above), a M_{c}-SO₂—NRₑ— group (wherein M_{c} and Rₑ are as defined above) or a M_{c}RₑN—SO₂— group (wherein M_{c} and Rₑ are as defined above), and

R_{d} is as defined above;

(3) the Z group: a —N═C(Yₐ)—Yₐ′— group (wherein Yₐ represents a hydrogen atom, or a C1-C10 alkyl group optionally substituted with a halogen atom, or a C1-C10 alkoxy group, and Yₐ′ represents an oxy group, or a thio group, or an imino group optionally substituted with a C1-C10 alkyl group),

a —Y_{b}—Y_{b}′—Y_{b}″— group (wherein Y_{b} and Y_{b}″ are the same or different, and represent a methylene group, or an oxy group, or a thio group, or a sulfinyl group, or an imino group optionally substituted with a C1-C10 alkyl group, and Y_{b}′ represents a C1-C4 alkylene group optionally substituted with a halogen atom, or a C1-C4 alkylene group optionally substituted with an oxo group), or

a —Y_{c}—O—Y_{c}′—O— group (wherein Y_{c} and Y_{c}′ are the same or different, and represent a C1-C10 alkylene group);

IV. B is

a group represented by formula (IV-1):

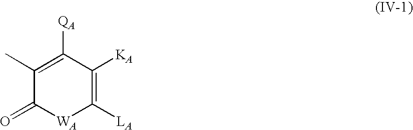

wherein,

(1) Q_{A} represents a hydroxyl group, a (b)- group (wherein (b) is as defined above), an A₉-B₆—B_{c}— group [wherein A₉ and B₆ are as defined above, and B_{c} represents an oxy group or a —N((O)ₘR₁)— group (wherein m and R₁ are as defined above), provided that B_{c} is not a sulfonyl group when A₉ is a hydrogen atom], an A₇″-SO₂—B_{c}— group (wherein A₇″ and B_{c} are as defined above), an A₈-SO₂—B_{c}— group (wherein A₈ and B_{c} are as defined above, provided A_{B} is not a hydrogen atom), a R₁R₁′N—SO₂—B_{c}— group (wherein R₁, R₁′ and B_{c} are as defined above), a (b₀)-SO₂—O— group (wherein (b) and B_{c} are as defined above), an A₉′B_{c}— group (wherein A₉′ and B_{c} are as defined above), a D₅-R₄—B_{c} group (wherein D₅, R₄ and B_{c} are as defined above), a M_{c}-B₃—B_{c}— group (wherein M_{c}, B₃ and B_{c} are as defined above) or a M_{c}-B_{c}— group (wherein M_{c} and B_{c} are as defined above),

(2) W_{A} represents an oxygen atom or a —NT_{A}- group [wherein T_{A} represents a hydrogen atom, an A₉′— group (wherein A₉′ is as defined above), a D₅-R₄— group (wherein D₅ and R₄ are as defined above) or a M_{c}- group (wherein M_{c} is as defined above)], and

(3) K_{A} and L_{A} form a —V_{A}═V_{A}′—V_{A}′″═V_{A}′″— group {wherein V_{A}, V_{A}′, V_{A}′ and V_{A}′″ are the same or different, and represent a methine group optionally substituted with a Mₐ- group (wherein Mₐ is as defined above), or a —N═ group, and at least one of V_{A}, V_{A}′, V_{A}″ and V_{A}′″ represents a —N═ group};

a group represented by formula (IV-2):

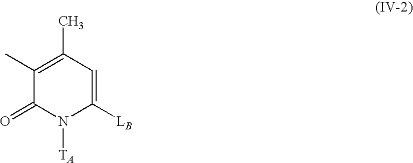

wherein T_{A} is as defined above, and L_{B} represents a hydroxyl group or a methyl group;

a group represented by formula (IV-3):

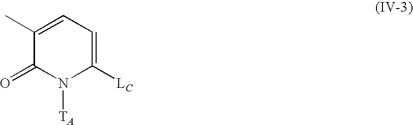

wherein T_{A} is as defined above, and L_{C} represents a C1-C10 alkyl group;

a group represented by formula (IV-4):

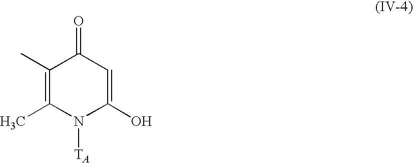

wherein T_{A} is as defined above;

a group represented by formula (IV-5):

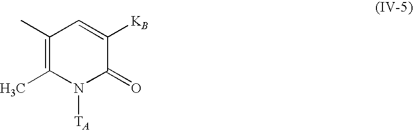

wherein T_{A} is as defined above, and K_{B} represents a cyano group or a UOCO— group (wherein U represents a hydrogen atom or C1-C10 alkyl group);

a group represented by formula (IV-6):

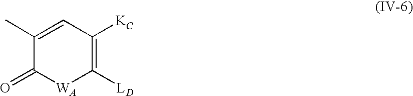

wherein W_{A} is as defined above, and K_{C} and L_{D} form a C3-C10 alkylene group, or a C4-C10 alkenylene group optionally substituted with 1 or more same or different Mₐ- groups (wherein Mₐ is as defined above);

a group represented by formula (IV-7):

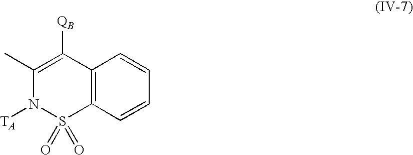

wherein T_{A} is as defined above, Q_{B} represents a hydroxyl group, an A₉-B₆—O— group [wherein A₉ and B₆ are as defined above], an A₇″-SO₂—O— group (wherein A₇″ is as defined above), an A₈-SO₂—O— group (wherein A₈ is as defined above, provided that A₈ is not a hydrogen atom), a R₁R₁′N—SO₂—O— group (wherein R₁ and R₁′ are as defined above), a (b)-SO₂—O— group (wherein (b) is as defined above), an A₉′-O— group (wherein A₉′ is as defined above), a D₅-R₄—O— group (wherein D₅ and R₄ are as defined above), a M_{c}-B₃—O— group (wherein M_{c} and B₃ are as defined above), or a M_{c}-O— group (wherein M_{c} is as defined above); or

a group represented by formula (IV-8)

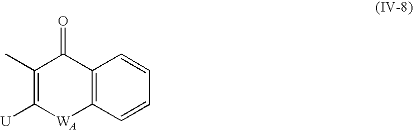

wherein U and W_{A} are as defined above; and

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range];

5. A cinnamoyl compound represented by the formula (V):

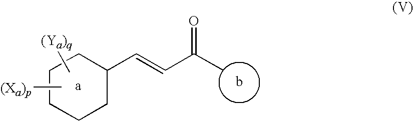

wherein,

I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a₁ represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)ₙ—CONH—CH₂— group (wherein r₀ represents a C1-C10 alkyl group, and l represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), a C1-C10 alkyl group substituted with a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,

Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring;

II. b is

a group represented by formula (V-1):

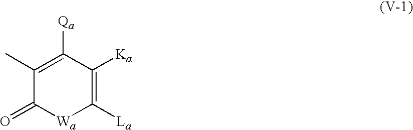

wherein Qₐ represents a rₐ-O— group {rₐ represents a hydrogen atom, or a C1-C10 alkyl group, or a C3-C10 alkenyl group, or a C3-C10 alkynyl, or a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r₁ is as defined above)}, or a piperidino group, or a morpholino group, or a r₄r₄′N-group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein Tₐ represents a r_{b}- group (wherein r_{b} is the same as or different from rₐ, and represents the same meaning as rₐ has) or a r₃′— group (wherein r₃′ is the same as or different from r₃, and represents the same meaning as r₃ has)], and Kₐ and Lₐ form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′Vₐ″ and Vₐ′″ represents a —N═ group);

a group represented by formula (V-2):

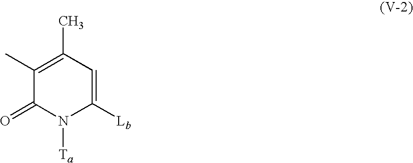

wherein Tₐ is as defined above, and L_{b} represents a hydroxyl group or a methyl group;

a group represented by formula (V-3):

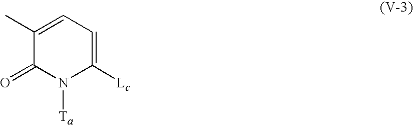

wherein Tₐ is as defined above, and L_{c} represents a C1-C10 alkyl group;

a group represented by formula (V-4):

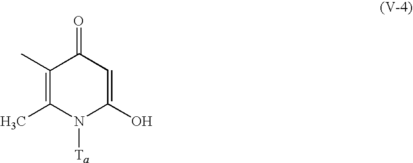

wherein Tₐ is as defined above;

a group represented by formula (V-5):

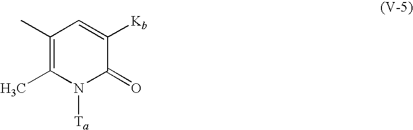

wherein Tₐ is as defined above, and K_{b} represents a cyano group or a UOCO— group (wherein U represents a hydrogen atom or C1-C10 alkyl group);

a group represented by formula (V-6):

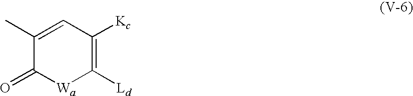

wherein Wₐ is as defined above, and K_{c} and L_{d} form a C3-C10 alkylene group or a C4-C10 alkenylene group;

a group represented by formula (V-7):

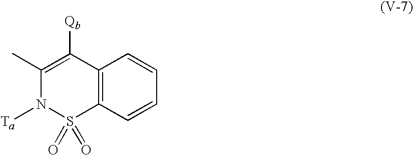

wherein Tₐ is as defined above, and Q_{b} represents a rₐ-O— group (wherein rₐ is as defined above); or

a group represented by formula (V-8):

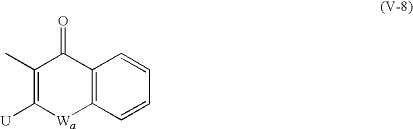

wherein U and Wₐ are as defined above; and

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

6. A process for producing a cinnamoyl compound represented by formula (VI′):

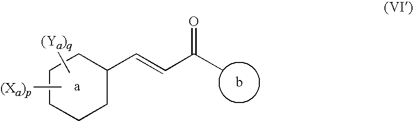

wherein a, Xₐ, Yₐ, p and q are defined below, and

b represents a group represented by formula (VI′-1):

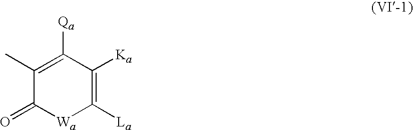

wherein Qₐ, Wₐ, Kₐ and Lₐ are as defined below, a group represented by formula (VI′-2):

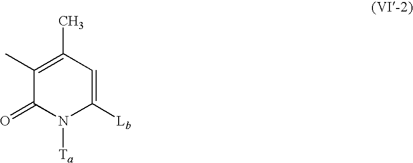

wherein Tₐ and L_{b} are as defined below, a group represented by formula (VI′-3):

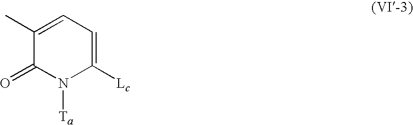

wherein Tₐ and L_{c} are as defined below, a group represented by formula (VI′-4):

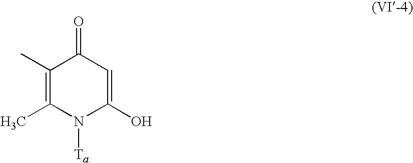

wherein Tₐ is as defined below, a group represented by formula (VI′-5):

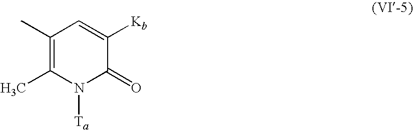

wherein Tₐ and K_{b} are as defined below, a group represented by formula (VI′-6):

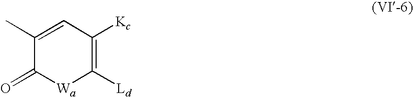

wherein Wₐ, K_{c} and L_{d} are as defined below, a group represented by formula (VI′-7):

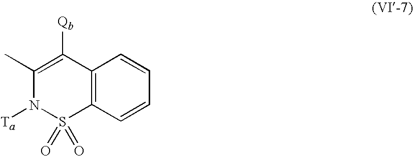

wherein Tₐ and Q_{b} are as defined below, or a group represented by formula (VI′-8):

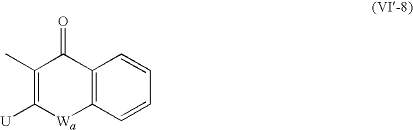

wherein U and Wₐ are as defined below; which comprises reacting a benzaldehyde derivative represented by the formula (VI):

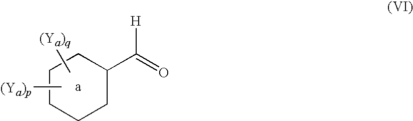

[wherein,

I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a₁ represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)ₗ—CONH—CH₂— group (wherein r₀ represents a C1-C10 alkyl group, and l represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), or a C1-C10 alkyl group substituted with a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,

Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring] with a compound represented by formula (VI-1):

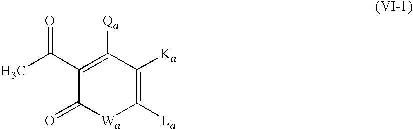

[wherein

Qₐ represents a rₐ-O— group {rₐ represents a hydrogen atom, or a C1-C10 alkyl group, or a C3-C10 alkenyl group, or a C3-C10 alkynyl, a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r₁ is as defined above)}, or a piperidino group, or a morpholino group, or a r₄r₄′N-group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein Tₐ represents a r_{b}- group (wherein r_{b} is the same as or different from rₐ, and represents the same meaning as rₐ has) or a r₃′— group (wherein r₃′ is the same as or different from r₃, and represents the same meaning as r₃ has)], and Kₐ and Lₐ form a —Vₐ═Vₐ′—Vₐ″═Vₐ′″— group (wherein Vₐ, Vₐ″, Vₐ′″ and Vₐ′″ are the same or different, and represent a methine group or a —N═ group, and at least one of Vₐ, Vₐ′Vₐ″ and Vₐ′″ represents a —N═ group)], a compound represented by formula (VI-2):

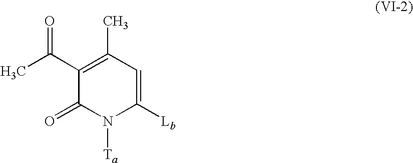

[wherein Tₐ is as defined above, and L_{b} represents a hydroxyl group or a methyl group], a compound represented by formula (VI-3):

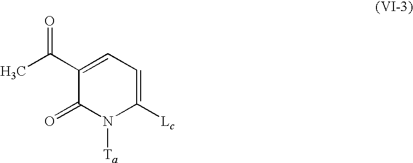

[wherein Tₐ is as defined above, and L_{c} represents a C1-C10 alkyl group], a compound represented by formula (VI-4):

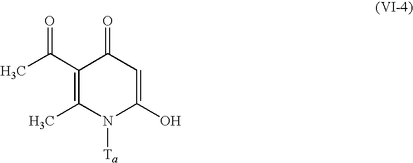

[wherein Tₐ is as defined above], a compound represented by formula (VI-5):

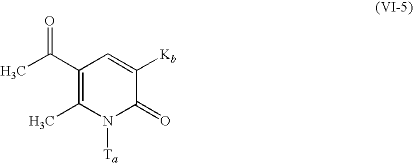

[wherein Tₐ is as defined above, and K_{b} represents a cyano group or a UOCO— group (wherein U represents a hydrogen atom or C1-C10 alkyl group)], a compound represented by formula (VI-6):

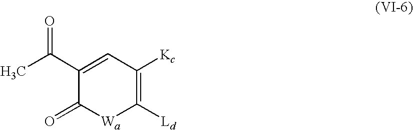

[wherein Wₐ is as defined above, and K_{c} and L_{d} form a C3-C10 alkylene group or a C4-C10 alkenylene group], a compound represented by formula (VI-7):

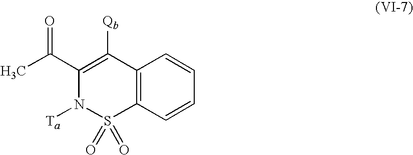

[wherein Tₐ is as defined above, and Q_{b} represents a rₐ-O— group (wherein rₐ is as defined above)], or a compound represented by formula (VI-8):

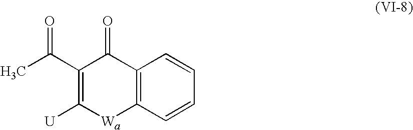

[wherein U and Wₐ are as defined above]; and

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

7. A process for producing a compound represented by formula (VII-3):

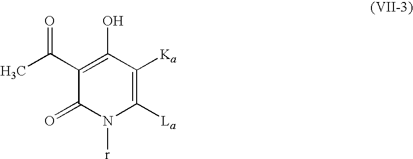

[wherein r, Kₐ and Lₐ are as defined below] which comprises 3-oxobutyrylamidating a compound represented by formula (VII-1):

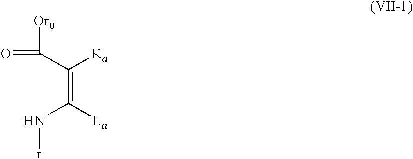

[wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group)] to produce a compound represented by formula (VII-2):

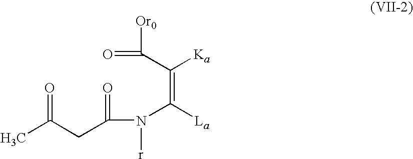

[wherein r, r₀, Kₐ and Lₐ are as defined above], and then cyclizing the compound represented by the formula (VII-2);
8. A process for producing a compound represented by formula (VIII-2):

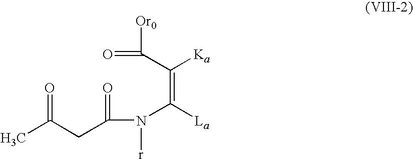

[wherein r, r₀, Kₐ and Lₐ are as defined below] which comprises 3-oxobutyrylamidating a compound represented by formula (VIII-1):

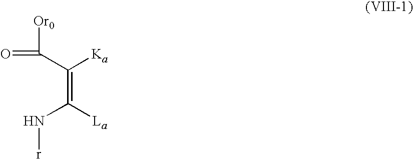

[wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group)];
9. A compound represented by the formula (IX):

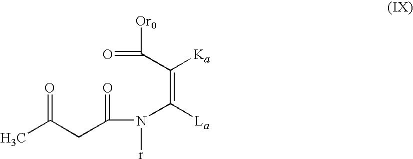

wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group);
10. A compound represented by the formula (X):

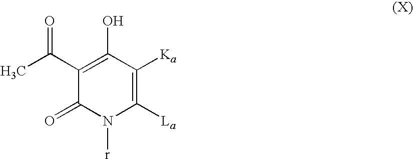

wherein r represents a hydrogen atom or a C1-C10 alkyl group, Kₐ and Lₐ form a —Vₐ═Vₐ′—Vₐ″═Vₐ′″— group (wherein Vₐ, Vₐ′, Vₐ″ and Vₐ′″ are the same or different and represent a methine group or a —N═ group, and at least one of Vₐ, Vₐ′, Vₐ″ and Vₐ′″ represents a —N═ group);
11. A process for producing a compound represented by formula (XI-2):

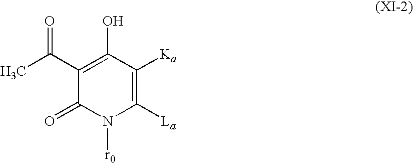

[wherein r₀ represents a C1-C10 alkyl group, and Kₐ and Lₐ are as defined below] which comprises alkylating a compound represented by formula (XI-1):

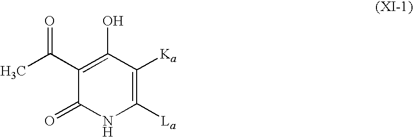

[wherein Kₐ and Lₐ form a —Vₐ═Vₐ′—Vₐ′″═Vₐ′″— group (wherein Vₐ, Vₐ′, Vₐ″ and Vₐ′″ are the same or different, and represent a methine group or a —N═ group, and at least one of Vₐ, Vₐ′, Vₐ″ and Vₐ′″ represents a —N═ group)];
12. A composition for suppressing transcription of an extracellular matrix gene, which comprises an inert carrier and a cinnamoyl compound represented by formula (I′):

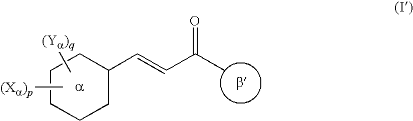

wherein,

I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;

(1) the X₀ group: a M_{α}-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ′″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;

(2) the Y₀ group: a M_{b0}-R_{d}— group, wherein M_{b0} represents a M_{c0}- group

[wherein M_{co} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a M_{α}-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by

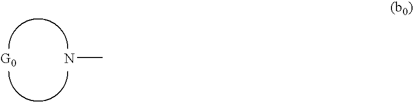

(wherein G₀ forms an optionally substituted, saturated or unsaturated, nonaromatic 5 to 14-membered hydrocarbon ring or heterocyclic ring), a (c₀)- group represented by

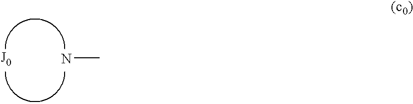

(wherein, J₀ forms a 5 to 7-membered aromatic ring optionally containing a nitrogen atom), a (d₀)- group represented by

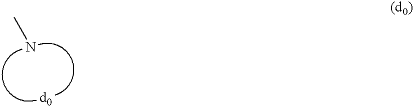

[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group}, a sulfinyl group, or a sulfonyl group], or a (e₀)- group represented by

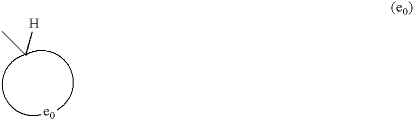

{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group); and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}-CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ group (wherein M_{c0} and Rₑ are as defined above), a M_{c0}RₑN—CO group (wherein M_{c0} and Rₑ are as defined above), a M_{c0}RₑN NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;

(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and

II. β′ represents

a group represented by formula (I′-1):

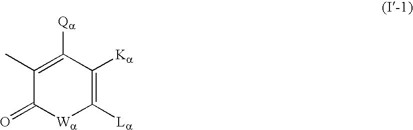

wherein,
(1) Q_{α} represents an optionally substituted hydroxyl group, or an optionally substituted amino group,
(2) W_{α} represents an oxygen atom or a —NT_{α}— group (wherein T_{α} represents a hydrogen atom, or a substituent on the nitrogen atom),
(3) K_{α} and L_{α} form a —V_{α}═V_{α}′—V_{α}″═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (I′-2):

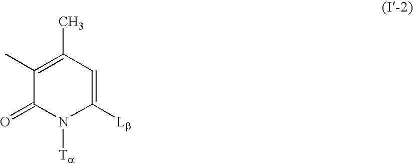

wherein T_{α} is as defined above, and L_{β} represents a hydroxyl group or a methyl group;
a group represented by formula (I′-3):

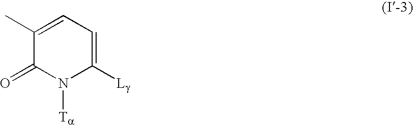

wherein T_{α} is as defined above, and L_{γ} represents a C1-C10 alkyl group;
a group represented by formula (I′-4):

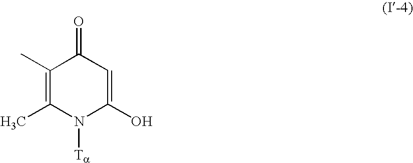

wherein T_{α} is as defined above;
a group represented by formula (I′-5):

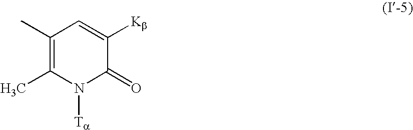

wherein T_{α} is as defined above, and K_{β} represents a cyano group or a UOCO— group (wherein U represents a hydrogen atom or a C1-C10 alkyl group);
a group represented by formula (I′-6):

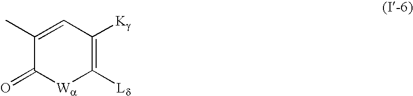

wherein W_{α} is as defined above, and K_{γ} and L_{δ} form an optionally substituted C3-C10 alkylene group or an optionally substituted C4-C10 alkenylene group;
a group represented by formula (I′-7):

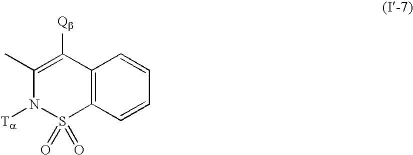

wherein T_{α} is as defined above, and Q_{β} represents an optionally substituted hydroxyl group; or
a group represented by formula (I′-8):

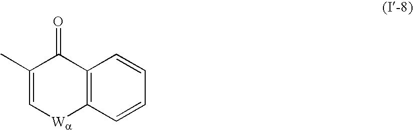

wherein W_{α} is as defined above;

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

13. A cinnamoyl compound represented by formula (II′):

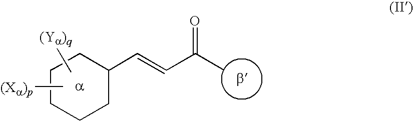

wherein,

I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;

(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}; Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ″—R_{d}— group (wherein Rₑ, Rₑ′, Rₑ″ and Rₑ′″ are the same or different, Rₑ, Rₑ′ and Rₑ″ are as defined above, Rₑ′ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ″ and R_{d} are as defined above), a C2-C10 alkenyl group or a C2-C10 alkynyl group;

(2) the Y₀ group: a M_{b0}-R_{d}— group, wherein M_{b0} represents a M_{c0}- group

[wherein M_{co} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by

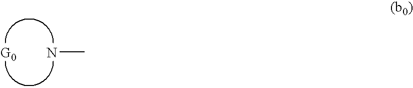

(wherein G₀ forms an optionally substituted, saturated or unsaturated, nonaromatic 5 to 14-membered hydrocarbon ring or heterocyclic ring), a (c₀)- group represented by

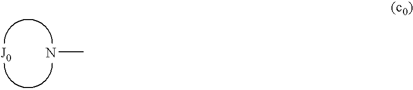

(wherein, J₀ forms a 5 to 7-membered aromatic ring optionally containing a nitrogen atom), a (d₀)- group represented by

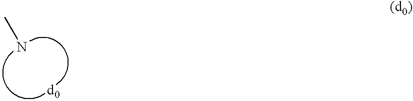

[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group), a sulfinyl group, or a sulfonyl group], or a (e₀)- group represented by

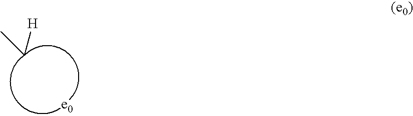

{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}; and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}-CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ group (wherein M_{c0} and Rₑ are as defined above), a M_{c0}RₑN—CO—group (wherein M_{c0} and Rₑ are as defined above), a M_{c0}RₑN—CO NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;

(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and

II. β′ represents

a group represented by formula (II′-1):

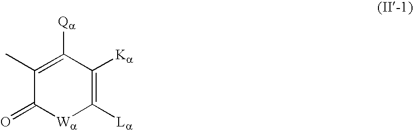

wherein,
(1) Q_{α} represents an optionally substituted hydroxyl group, or an optionally substituted amino group,
(2) W_{α} represents an oxygen atom or a —NTₐ- group (wherein T_{α} represents a hydrogen atom, or a substituent on the nitrogen atom),
(3) K_{α} and L_{α} form a —V_{α}═V_{α}′—V_{α}″═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (II′-2):

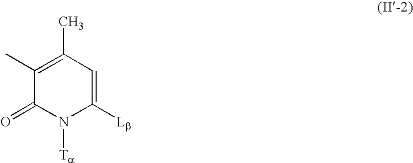

wherein T_{α} is as defined above, and L_{β} represents a hydroxyl group or a methyl group;
a group represented by formula (II′-3):

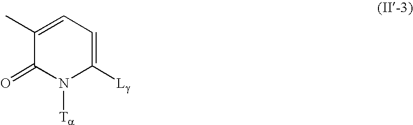

wherein T_{α} is as defined above, and L_{γ} represents a C1-C10 alkyl group;
a group represented by formula (II′-4):

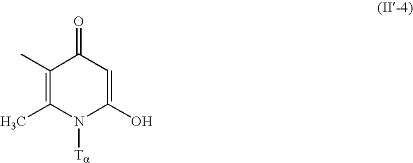

wherein T_{α} is as defined above;
a group represented by formula (II′-5):

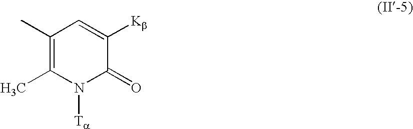

wherein T_{α} is as defined above, and K_{β} represents a cyano group or a UOCO— group (wherein U represents a hydrogen atom or a C1-C10 alkyl group);
a group represented by formula (II′-6):

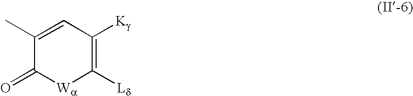

wherein W_{α} is as defined above, and K_{γ} and L_{δ} form an optionally substituted C3-C10 alkylene group or an optionally substituted C4-C10 alkenylene group; a group represented by formula (II′-7):

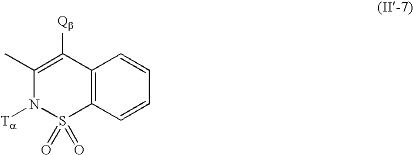

wherein T_{α} is as defined above, and Q_{β} represents an optionally substituted hydroxyl group; or
a group represented by formula (III-8):

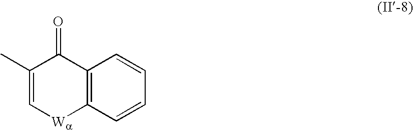

wherein W_{α} is as defined above;

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

14. A cinnamoyl compound represented by formula (V′):

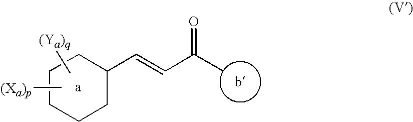

[wherein,

I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a₁ represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)ₙ—CONH—CH₂— group (wherein r₀ represents a C1-C10 alkyl group, and 1 represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), or a C1-C10 alkyl group substituted with a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,

Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring;

II. b is

a group represented by formula (V′-1):

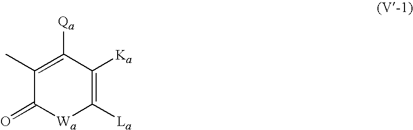

wherein Qₐ represents a rₐ-O— group {rₐ represents a hydrogen atom, or a C1-C10 alkyl group, or a C3-C10 alkenyl group, or a C3-C10 alkynyl, or a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r, is as defined above)}, or a piperidino group, or a morpholino group, or a r₄r₄′N-group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein T_{α} represents a r_{b}- group (wherein r_{b} is the same as or different from rₐ, and represents the same meaning as rₐ has) or a r₃′- group (wherein r₃′ is the same as or different from r₃, and represents the same meaning as r₃ has)], and Kₐ and Lₐ form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′Vₐ″ and Vₐ′″ represents a —N═ group);

a group represented by formula (V′-2):

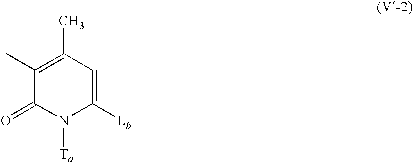

wherein Tₐ is as defined above, and L_{b} represents a hydroxyl group or a methyl group;

a group represented by formula (V′-3):

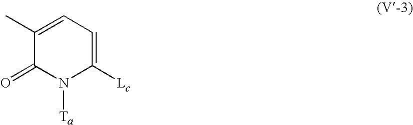

wherein Tₐ is as defined above, and L_{c} represents a C1-C10 alkyl group;

a group represented by formula (V′-4):

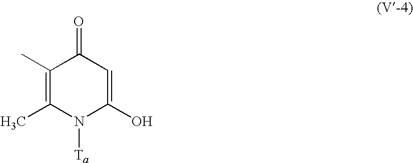

wherein Tₐ is as defined above;

a group represented by formula (V′-5):

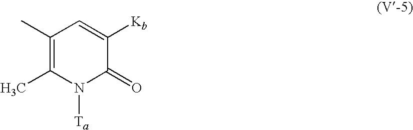

wherein Tₐ is as defined above, and K_{b} represents a cyano group or a UOCO— group (wherein U represents a hydrogen atom or C1-C10 alkyl group);

a group represented by formula (V′-6):

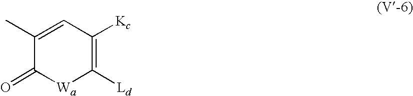

wherein Wₐ is as defined above, and K_{c} and L_{d} form a C3-C10 alkylene group or a C4-C10 alkenylene group;

a group represented by formula (V′-7):

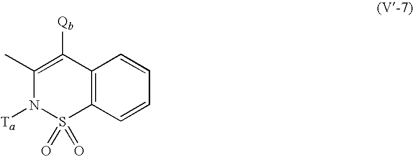

wherein Tₐ is as defined above, and Q_{b} represents a rₐ-O— group (wherein rₐ is as defined above); or

a group represented by formula (V′-8):

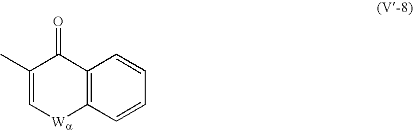

wherein Wₐ is as defined above; and

the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range;

15. A composition for suppressing transcription of an extracellular matrix gene, which comprises the compound according to any one of the above items 2 to 5, 13 and 14, and an inert carrier;
16. A composition for suppressing transcription of an extracellular matrix gene, which comprises the compound according to the above item 5 or 14, and an inert carrier;
17. Use of a cinnamoyl compound contained in the composition according to the above item 1 or 12 as an active ingredient, as an active ingredient for suppressing transcription of an extracellular matrix gene;
18. Use of the compound according to any one of the above items 2 to 5, 13 and 14, as an active ingredient for suppressing transcription of an extracellular matrix gene;
19. Use of the compound according to the above item 5 or 14, as an active ingredient for suppressing transcription of an extracellular matrix gene;
20. Use of a cinnamoyl compound contained in the composition according to the above item 1 or 12 as an active ingredient, as an active ingredient for decreasing expression of an extracellular matrix gene to induce a reduction in accumulation of an extracellular matrix and thereby improving tissue fibrosis;
21. Use of the compound according to any one of the above items 2 to 5, 13 and 14, as an active ingredient for decreasing expression of an extracellular matrix gene to induce a reduction in accumulation of an extracellular matrix and thereby improving tissue fibrosis;
22. A method for improving tissue fibrosis, which comprises administering an effective amount of a cinnamoyl compound contained in the composition according to the above item 1 or 12 as an active ingredient to a mammalian patient in need thereof;
23. A method for improving tissue fibrosis, which comprises administering an effective amount of the compound according to any one of the above items 2 to 5, 13 and 14 to a mammalian patient in need thereof;
24. An agent for treating chronic renal failure, which comprises the compound according to any one of the above items 2 to 5, 13 and 14 and an inert carrier;
25. Use of a cinnamoyl compound contained in the composition according to the above item 1 or 12 as an active ingredient, or the compound according to any one of the above items 2 to 5, 13 and 14, as an active ingredient for treating chronic renal failure;
26. A method for treating chronic renal failure, which comprises administering an effective amount of a cinnamoyl compound contained in the composition according to the above item 1 or 12 as an active ingredient, or the compound according to any one of the above items 2 to 5, 13 and 14 to a mammalian patient in need thereof;
27. An agent for treating heart failure, which comprises a cinnamoyl compound contained in the composition according to the above item 1 or 12 as an active ingredient, or the compound according to any one of the above items 2 to 5, 13 and 14, and an inert carrier;
28. Use of a cinnamoyl compound contained in the composition according to the above item 1 or 12 as an active ingredient, or the compound according to any one of the above items 2 to 5, 13 and 14, as an active ingredient for treating heart failure;
29. A method for treating heart failure, which comprises administering an effective amount of a cinnamoyl compound contained in the composition according to the above item 1 or 12 as an active ingredient, or the composition according to any one of the above items 2 to 5, 13 and 14 to a mammalian patient in need thereof;
30. A composition for suppressing the activity of TGF-β, which comprises a cinnamoyl compound contained in the composition according to the above item 1 or 12 as an active ingredient, and an inert carrier;
31. A composition for suppressing the activity of TGF-β, which comprises the compound according to any one of the above items 2 to 5, 13 and 14, and an inert carrier;
32. Use of a cinnamoyl compound contained in the composition according to the above item 1 or 12 as an active ingredient, as an active ingredient for suppressing the activity of TGF-β;
33. Use of the compound according to any one of the above items 2 to 5, 13 and 14, as an active ingredient for suppressing the activity of TGF-β;
34. A composition for hair growth which comprises a cinnamoyl compound contained in the composition according to the above item 1 or 12 as an active ingredient, or the compound according to any one of the above items 2 to 5, 13 and 14, and an inert carrier;
35. Use of a cinnamoyl compound contained in the composition according to the above item 1 or 12 as an active ingredient, or the compound according to any one of the above items 2 to 5, 13 and 14, as an active ingredient for inhibiting a promoting effect of TGF-β on transition to a hair regression phase to induce extension of a hair growth phase and thereby providing a hair-growing effect;
36. A method for growing hair, which comprises administering an effective amount of a cinnamoyl compound contained in the composition according to the above item 1 or 12 as an active ingredient, or the compound according to any one of the above items 2 to 5, 13 and 14, to a mammalian patient in need thereof; and the like.

### BEST MODE FOR CARRYING OUT THE INVENTION

The present invention will be explained in detail below.

In the present invention, a saturated hydrocarbon group in an alkyl group, a haloalkyl group, an alkoxy group, an alkoxycarbonyl group, an alkylthio group, an alkylsulfinyl group, an alkylsulfonyl group and an alkylene group may be branched, or a part or all of carbon atoms thereof may form a ring. An unsaturated hydrocarbon group in an alkenyl group, an alkenyloxy group, an alkynyl group, an alkynyloxy group, an alkenylene group and an alkynylene group may be branched, or a part or all of carbon atoms thereof may form a ring, and the number of unsaturated bonds thereof may be singular or plural.

In the present invention, examples of an alkyl group include a methyl group, an ethyl group, an isopropyl group, a cyclohexyl group, a cyclopropylmethyl group and the like. Examples of a haloalkyl group include a 2,2,2-trifluoroethyl group and the like. Examples of an alkoxy group include a methoxy group, an ethoxy group, a cyclopentyloxy group, a 2-cyclohexylethoxy group and the like. Examples of an alkylthio group include a methylthio group and the like. Examples of an alkylsulfinyl group include a methylsulfinyl group and the like. Examples of an alkylsulfonyl group include a methylsulfonyl group and the like. Examples of an alkylene group include a methylene group, an ethylethylene group, a 1,4-cyclohexylene group and the like. Examples of an alkenyl group include a vinyl group, a 2-propenyl group, a 3-methyl-2-butenyl group, a 1,3-butadienyl group, a 3-cyclohexenyl group and the like. Examples of an alkynyl group include an ethynyl group, a 2-propynyl group, a 2-penten-4-ynyl group and the like. Examples of an alkenylene group include a vinylene group, a propenylene group, a 1,3-butadienylene group and the like. Examples of an alkynylene group include an ethynylene group, a propynylene group and the like.

In the present invention, examples of a halogen atom include a fluorine atom, a chlorine atom, a bromine atom and an iodine atom.

In the present invention, a pyridyl group includes a 2-pyridyl group, a 3-pyridyl group and a 4-pyridyl group. A furyl group includes a 2-furyl group and a 3-furyl group. A thienyl group includes a 2-thienyl group and a 3-thienyl group. A naphthyl group includes a 1-naphthyl group and a 2-naphthyl group.

In cinnamoyl compounds represented by the formulas (I), (II), (III), (IV), (V), (VI), (VI′), (I′), (II′) and (V′) (hereinafter, referred to as the compounds (I), (II), (III), (IV), (V), (VI), (VI′), (I′), (II′) and (V′), respectively, in some cases), when the α ring, the A0 ring, the A ring or the a ring is a pyridine ring, an N-oxide thereof is also included.

The compounds (I) to (V), (I′), (II′) or (V′) (hereinafter referred to as present compound in some cases) includes a pharmaceutically acceptable salt thereof. The pharmaceutically acceptable salt includes a salt of the compound (I) to (V), (I′) (II′) or (V′) with an inorganic acid, an organic acid, an inorganic base, or an organic base. Examples of a salt with an inorganic acid include hydrochloride, hydrobromide and the like. Examples of a salt with an organic acid include acetate, benzoate and the like. Examples of a salt with an inorganic base include a potassium salt, a sodium salt and the like. Examples of a salt with an organic base include a pyridine salt, a morpholine salt and the like.

X_{A0}, Y_{A0}, G_{A0}, K_{A0}, L_{A0} and T_{A0} in the compound (III) are independently represented by a group represented by D₁, D₂, D₃, D₄, D₅, R₀, R₀′, R₀″, R₁, R₁′, R₁″, R₂, R₂′, R₃, R₄, R₄′, R₅, R₆, A₁, A₂, A₃, A₄, A₅, A₆, A₇, A₇′, A₇″, A₈, A₈′, A₉, A₉′, A₉″, A₁₀, A₁₀′, A₁₁, B, B′, B₀, B₁, B₁′, B₂, B₂′, B₃, B₃′, B₄, B₄′, B₅, B₆, (a₀), (b₀), (c₀), (d₀), (e₀), Mₐ, Mₐ′, Mₐ″, Mₐ′″ Mₐ″″, M_{b0}, Mc₀, Md₀, Ra₀, R_{b}, R_{c}, R_{d}, R_{d}′, Rₑ, Rₑ′, Rₑ″, Rₑ′″, Bₐ, B_{b}, B_{c}, Yₐ, Yₐ′, Y_{b}, Y_{b}′, Y_{b}″, Y_{c} and Y_{c}′, and an integer represented by k, k′, l, m, m′, n and n′.

X_{A}, Y_{A}, Q_{A}, K_{A}, L_{A} and T_{A} in the compound (IV) are independently represented by a group represented by D₁, D₂, D₃, D₄, D₅, R₀, R₀′, R₀″, R₁, R₁′, R₁″, R₂, R₂′, R₃, R₄, R₄′, R₅, R₆, A₁, A₂, A₃, A₄, A₅, A₆, A₇, A₇′, A₇″, A₈, A₈′, A₉, A₉′, A₉″, A₁₀, A₁₀′, A₁₁, B, B′, B₀, B₁, B₁′, B₂, B₂′, B₃, B₃′, B₄, B₄′, B₅, B₆, (a), (b), (c), (d), (e), Mₐ, Mₐ′, Mₐ″, Mₐ′″, Mₐ″″, M_{b}, M_{c}, M_{d}, Rₐ, R_{b}, R_{c}, R_{d}, R_{d}′, Rₑ, Rₑ′, Rₑ″, Rₑ′″, Bₐ, B_{b}, B_{c}, Yₐ, Yₐ′, Y_{b}, Y_{b}′, Y_{b}″, Y_{c} and Y_{c}′, and an integer represented by k, k′, l, m, m′, n and n′.

Xₐ, Yₐ, qₐ and tₐ in compounds (V) and (V′) are independently represented by a group represented by a₀, a₀′, a₁, a₁′, a₂, a₃, a₄, a₅, b, b′, r, r′, r₀, r₀′, r₁, r₁′, r₂, r₃, r₃′, r₄, r₄′, rₐ, r_{b}, y and z, and an integer represented by l.

The “6- to 10-membered aryl group”, in the Y₀ group of substituents which may be selected as Y_{α} of the compounds (I), (II), (I′) and (II′), represents a group forming a monocyclic or fused aromatic hydrocarbon ring, and examples thereof include a phenyl group, a 1-naphthyl group, a 2-naphtyl group, a 6-indanyl group and the like. The “5- to 10-membered heteroaryl group” represents a group forming a monocyclic or fused aromatic heterocycle, and examples thereof include a 2-furyl group, a 3-furyl group, a 2-thienyl group, a 3-thienyl group, a 2-pyridyl group, a 3-pyridyl group, a 4-pyridyl group, a 2-quinolyl group, and the like. The “3- to 10-membered cyclic hydrocarbon or heterocyclic group optionally containing an unsaturated bond” includes a monocyclic or fused ring, and examples thereof include a 2-cyclohexenyl group, a 2-morpholinyl group, a 4-piperidyl group and the like. The “3- to 10-membered cyclic hydrocarbon or heterocyclic group optionally containing an unsaturated bond” may be substituted with 1 or more same or different Mₐ-groups as describe above.

The “group which is fused to the α ring”, in the Z₀ group of substituents which may be selected as Y_{α} of the compounds (I), (II), (I′) and (II′), may have 1 or more same or different atoms or groups selected from a halogen atom, a C1-C10 alkoxy group, a C3-C10 alkenyloxy group, a C3-C10 alkynyloxy group, a carbonyl group, a thiocarbonyl group, an oxy group, a thio group, a sulfinyl group and a sulfonyl group.

In Rₐ₀ of the E₀ group of substituents which may be selected as X_{A0} of the compound (III), the “optionally substituted 5- to 7-membered aryl group or heteroaryl group” represents a group forming a monocyclic or fused aromatic hydrocarbon ring or a group forming a monocyclic or fused aromatic heterocycle, and examples thereof include a phenyl group, a 1-naphthyl group, a 2-naphthyl group, a 6-indanyl group, a 2-furyl group, a 3-furyl group, a 2-thienyl group, a 3-thienyl group, a 2-pyridyl group, a 3-pyridyl group, a 4-pyridyl group, a 2-quinolyl group and the like. The “optionally substituted 5- to 7-membered aryl group or heteroaryl group” may be substituted with 1 or more same or different Mₐ-groups as described above.

In (d₀) of the Y₀ group of substituents which may be selected as Y_{α} or Y_{A0} of the compounds (I), (II), (III), (I′) and (II′), the phrase “forming a 5 to 12-membered hydrocarbon ring which is substituted with a carbonyl group or a thiocarbonyl group and further which may be optionally substituted with an oxy group, a thio group, a —NR₁— group (wherein R₁ is as defined above), a sulfinyl group or a sulfonyl group” means forming a 5- to 12-membered hydrocarbon ring in which one or more of carbon atoms are substituted with a carbonyl group or a thiocarbonyl group, and further, one or more of carbon atoms may be substituted with one or more same or different groups selected from an oxy group, a thio group, a —NR₁— group (wherein R₁ is as defined above), a sulfinyl group and a sulfonyl group.

In (e₀) of the Y₀ group of substituents which may be selected as Y_{α} or Y_{A0} of the compounds (I), (II), (III), (I′) and (II′), the phrase “forming a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group” means forming a 5- to 12-membered hydrocarbon ring in which one or more of carbon atoms are optionally substituted with one or more same or different groups selected from a carbonyl group, a thiocarbonyl group, an oxy group, a thio group, a —NR₁— group (wherein R₁ is as defined above), a sulfinyl group and a sulfonyl group.

In (a) of the B group of substituents which may be selected as X_{A} of the compound (IV), the “C2-C10 alkylene group optionally substituted with an oxy group, a thio group, a sulfinyl group, a sulfonyl group or a —NR₁′— group (wherein R₁′ is as defined above)” means a C2-C10 alkylene group in which one or more of carbon atoms may be substituted with one or more same or different groups selected from an oxy group, a thio group, a sulfinyl group, a sulfonyl group and a —NR₁′— group (wherein R₁′ is as defined above), and the “C3-C10 alkenylene group optionally substituted with an oxy group, a thio group, a sulfinyl group, a sulfonyl group or a —NR₁′— group (wherein R₁′ is as defined above)” means a C3-C10 alkenylene group in which one or more of carbon atoms may be substituted with one or more same or different groups selected from an oxy group, a thio group, a sulfinyl group, a sulfonyl group and a —NR₁′— group (wherein R₁′ is as defined above).

In (b) of the D group of substituents which may be selected as X_{A} of the compound (IV), the “C1-C10 alkylene group optionally substituted with a methyl group, an oxy group, a thio group, a sulfinyl group, a sulfonyl group or a —NR₁— group (wherein R₁ is as defined above)” means a C2-C10 alkylene group in which one or more of carbon atoms may be substituted with a methyl group, or one or more of carbon atoms may be substituted with one or more same or different groups selected from an oxy group, a thio group, a sulfinyl group, a sulfonyl group and a —NR₁— group (wherein R₁ is as defined above), and the “C2-C10 alkenylene group optionally substituted with a methyl group, an oxy group, a thio group, a sulfinyl group, a sulfonyl group or a —NR₁— group (wherein R₁ is as defined above)” means a C2-C10 alkenylene group in which one or more of carbon atoms may be substituted with a methyl group, or one or more carbon atoms may be substituted with one or more same or different groups selected from an oxy group, a thio group, a sulfinyl group, a sulfonyl group and a —NR₁— group (wherein R₁ is as defined above).

Examples of groups belonging to the X₀ group, the Y₀ group and the Z₀ group which may be selected as Y_{α} of the compounds (I), (II), (I′) and (II′) are shown in the following Table 24, Table 25 and Table 26, respectively.

Examples of groups belonging to the A₀ group, the Bo group, the C₀ group, the D₀ group, the E₀ group, the F₀ group, the G₀ group, the H₀ group, the I₀ group, the J₀ group, the K₀ group, the L₀ group, the M₀ group and the N₀ group which may be selected as X_{A0} of the compound (III) are shown in the following Table 1, Table 2, Table 3, Table 4, Tables 5 to 8, Tables 9 to 12, Tables 13 to 16, Table 17, Table 18, Table 19, Table 20, Table 21, Table 22 and Table 23, respectively. Examples of groups belonging to the X₀ group, the Y₀ group and the Z₀ group which may be selected as Y_{A0} of the compound (III) are shown in the following Table 24, Table 25 and Table 26. Examples of groups which may be selected as Q_{A0} and T_{A0} of the compound (III) are shown in the following Table 27 to Table 28 and Table 29, respectively.

Examples of groups belonging to the A group, the B group, the C group, the D group, the E group, the F group, the G group, the H group, the I group, the J group, the K group, the L group, the M group and the N group which may be selected as X_{A} of the compound (IV) are shown in the following Table 1, Table 2, Table 3, Table 4, Tables 5 to 8, Tables 9 to 12, Tables 13 to 16, Table 17, Table 18, Table 19, Table 20, Table 21, Table 22 and Table 23, respectively. Examples of groups belonging to the X group, the Y group and the Z group which may be selected as Y_{A} of the compound (IV) are shown in the following Table 24, Table 25 and Table 26, respectively. Examples of groups which may be selected as Q_{A} and T_{A} of the compound (IV) are shown in the following Table 27 to Table 28 and Table 29, respectively.

Examples of the aforementioned groups belonging to the A₀ group to N₀ group and the A group to N group are shown in the following Table 1 to Table 23. When said groups have geometrical isomers, all of the geometrical isomers are included, and when said groups have tautomers, all of the tautomers are included.

Examples of groups belonging to the A₀ group and the A group are shown in Table 1.

TABLE 1

| No. | Group |
|---|---|
| A-1 | —CH₂ONH₂ |
| A-2 | —CH₂ON(CH₃)₂ |
| A-3 | —CH₂ONHCOCH₃ |
| A-4 | —CH₂NHOCH₂CH═CH₂ |
| A-5 | —CH₂CN |
| A-6 | —CH₂CH₂CN |
| A-7 | —CH₂CH₂C(═NH)NH₂ |
| A-8 | —CH₂CH₂C(═NCH₂C≡CH)N(CH₃)₂ |
| A-9 | —CH₂C(═NH)NHCOCH₃ |
| A-10 | —CH₂C(═NOCOCH₃)—NH₂ |
| A-11 | —CH₂C(═NCOCH₃)—OCH₃ |
| A-12 | —CH₂CSNH₂ |
| A-13 | —CH₂NO₂ |
| A-14 | —CH₂SO₃H |
| A-15 | —SO₃H |

Examples of groups belonging to the B₀ group and the B group are shown in Table 2.

TABLE 2

| No. | Group |
|---|---|
| B-1 | 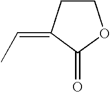 |
| B-2 | 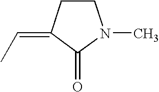 |
| B-3 | 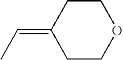 |
| B-4 | 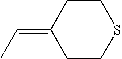 |
| B-5 | 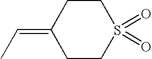 |
| B-6 | 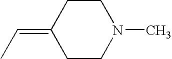 |

Examples of groups belonging to the CO group and the C group are shown in Table 3.

TABLE 3

| No. | Group |
|---|---|
| C-1 | —CH═CF₂ |
| C-2 | —CH═CHOCH₃ |
| C-3 | —CH═CHSCH₃ |
| C-4 | —CH═CHSOCH₃ |
| C-5 | —CH═CHSO₂CH₃ |
| C-6 | —CH═CHCH₂OH |
| C-7 | —CH═CHCH₂OCOCH₃ |
| C-8 | —CH═CHCHO |
| C-9 | —CH═CHCH═NCH₂CH═CH₂ |
| C-10 | —CH═CHCH═NOH |
| C-11 | —CH═CHCH═NOCH₂COOCH₃ |
| C-12 | —CH═CHCH═NOCH₂CN |
| C-13 | —CH═CHCH═NN(CH₃)₂ |
| C-14 | —CH═CHCH═NNHCOCH₃ |
| C-15 | —CH═CHCOCH₃ |
| C-16 | —CH═C(CH₃)COCH₃ |
| C-17 | —CH═CHCOCF₃ |
| C-18 | —CH═CHCH₂ON(CH₃)₂ |
| C-19 | —CH═CHCH₂ON(SO₂CH₃)CH₃ |
| C-20 | —CH═CHCH₂N(CH₂CH═CH₂)₂ |
| C-21 | —CH═CHCH₂N(OH)CH₃ |
| C-22 | —CH═CHNHCOCH₃ |
| C-23 | —CH═CHCN |
| C-24 | —CH═CHC(═NH)N(CH₃)₂ |
| C-25 | —CH═CHC(═NH)NHOCH₃ |
| C-26 | —CH═CHCSNH₂ |
| C-27 | —CH═CHNO₂ |
| C-28 | —CH═CHSO₃H |

Examples of groups belonging to the D₀ group and the D group are shown in Table 4.

TABLE 4

| No. | Group |
|---|---|
| D-1 | 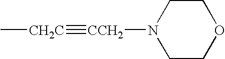 |
| D-2 | 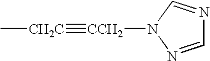 |
| D-3 | —C≡CI |
| D-4 | —C≡CCH₂SCH₃ |
| D-5 | —C≡CC(CH₃)₂OH |
| D-6 | —C≡CCH₂OCOOCH₃ |
| D-7 | —C≡CCH≡NCH₃ |
| D-8 | —C≡CCH≡NOCH₃ |
| D-9 | —C≡CCH≡NN(CH₃)₂ |
| D-10 | —C≡CCH₂ON(CH₃)₂ |
| D-11 | —C≡CCH₂N(CH₃)₂ |
| D-12 | —C≡CCH₂CH₂NO₂ |

Examples of groups belonging to the E₀ group and the E group are shown in Table 5 to Table 8.

TABLE 5

| No. | Group |
|---|---|
| E-1 | —CH═CHCOOCH₃ |
| E-2 | —CH═CHCOOC₂H₅ |
| E-3 | —CH═CHCOOCH₂CH₂Cl |
| E-4 | —CH═CHCOOCH₂CF₃ |
| E-5 | —CH═CHCOOCH₂CH═CH₂ |
| E-6 | —CH═CHCOOCH₂C≡CH |
| E-7 | 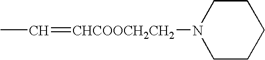 |
| E-8 | 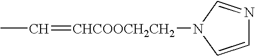 |
| E-9 | —CH═CHCOOCH₂CH₂OCH₃ |
| E-10 | —CH═CHCOOCH₂CH₂SCH₃ |
| E-11 | —CH═CHCOOCH₂CH₂SOCH₃ |
| E-12 | —CH═CHCOOCH₂CH₂SO₂CH₃ |
| E-13 | —CH═CHCOOCH₂CH₂OH |
| E-14 | —CH═CHCOOCH₂CH₂OSO₂N(CH₃)₂ |
| E-15 | —CH═CHCOOCH₂CH₂COCH₃ |
| E-17 | —CH═CHCOOCH₂CH₂ON(CH₃)₂ |
| E-18 | —CH═CHCOOCH₂CH₂N(CH₃)₂ |
| E-19 | —CH═CHCOOCH₂CH₂N(OC₂H₅)C₂H₅ |
| E-20 | —CH═CHCOOCH₂CH₂NRCOCH₃ |
| E-21 | —CH═CHCOOCH₂CH₂N(CH₃)COCH₃ |
| E-22 | —CH═CHCOOCH₂CH₂NHCOOCH₂CH₂OCH₃ |
| E-23 | —CH═CHCOOCH₂CH₂NHCOSCH₂CH═CH₂ |
| E-24 | —CH═CHCOOCH₂CH₂NHCONHC₂H₅ |
| E-25 | —CH═CHCOOCH₂CH₂NHCON(CH₃)₂ |

TABLE 6

| No. | Group |
|---|---|
| E-26 | —CH═CHCOOCH₂CH₂NHCON(OCH₃)CH₃ |
| E-27 | —CH═CHCOOCH₂CH₂NHCSNHCH₂CH₂Cl |
| E-28 | —CH═CHCOOCH₂CH₂NHSO₂N(CH₃)₂ |
| E-29 | —CH═CHCOOCH₂CH₂CN |
| E-30 | —CH═CHCOOCH₂CH₂NO₂ |
| E-31 | —CH═CHCOOCH₂CH₂SO₃H |
| E-32 | 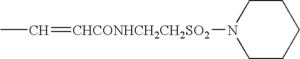 |
| E-33 | —CH═CHCONHCH₂CH₂SO₂N(CH₃)₂ |
| E-34 | —CH═CHCOSCH₃ |
| E-35 | —CH═CHCON(CH₃)CH₂C≡CH |
| E-36 | —CH═CHCON(OCH₃)CH₃ |
| E-37 | —CH═CHCONHOCH₃ |
| E-38 | —CH═CHCONHOCH₂CH═CH₂ |
| E-39 | —CH═CHCOOCH₂COOCH₃ |
| E-40 | —CH═CHCOSCH₂COOCH₃ |
| E-41 | —CH═CHCONHCH₂COOCH₃ |
| E-42 | —CH═CHCONHCH₂CON(CH₃)₂ |
| E-43 | —CH═CHCONHCH₂CN |
| E-44 | —CH═CHCONHCH₂C(═NH)N(CH₃)CH₂CH═CH₂ |
| E-45 | —CH═CHCONHCH₂C(═NH) NHOH |

TABLE 7

| No. | Group |
|---|---|
| E-46 | —CH═CHCONHSO₂CH₃ |
| E-47 | 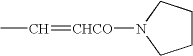 |
| E-48 | 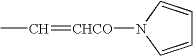 |
| E-49 | 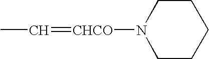 |
| E-50 | 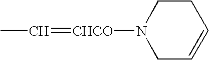 |
| E-51 | 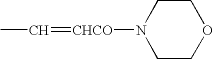 |
| E-52 | 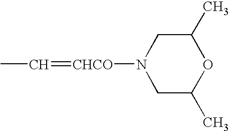 |
| E-53 | 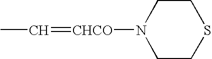 |
| E-54 | 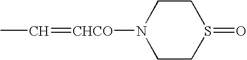 |
| E-55 | 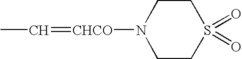 |

TABLE 8

| No. | Group |
|---|---|
| E-56 | 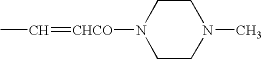 |
| E-57 | 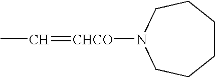 |
| E-58 | 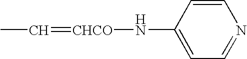 |
| E-59 | —CH═CHCONHN(CH₃)₂ |
| E-60 | —CH═CHCONHNHCOOC₂H₅ |
| E-61 | —CH═CHCONHNHCSNH(C)C₆H₁₁ |
| E-62 | —CH═CFCOOCH₃ |

Examples of groups belonging to the F₀ group and the F group are shown in Table 8 to Table 12.

TABLE 9

| No. | Group |
|---|---|
| F-1 | —OCH₂CH₂OH |
| F-2 | —OCH₂CH₂CH₂OH |
| F-3 | —CH₂OCH₂CH₂OH |
| F-4 | —OCH₂CH₂OCON(CH₃)₂ |
| F-5 | —OCH₂CH₂ONH₂ |
| F-6 | —OCH₂CH₂N(CH₃)₂ |
| F-7 | —OCH₂CH₂CH₂N(CH₃)₂ |
| F-8 | —OCH₂CH₂N(OCH₃) CH₃ |
| F-9 | —OCH₂CH₂NH₂ |
| F-10 | —OCH₂CH₂NHCOCH₃ |
| F-11 | —OCH₂CH₂N(CH₃) COCH₃ |
| F-12 | —OCH₂CH₂NHCOO(t)C₄H₉ |
| F-13 | —OCH₂CH₂NHCOSCH₂CH═CH₂ |
| F-14 | —OCH₂CH₂NHCONHC₂H₅ |
| F-15 | —OCH₂CH₂NHCON(CH₃)₂ |
| F-16 | —OCH₂CH₂NHCON(OCH₃) CH₃ |
| F-17 | —OCH₂CH₂NHCSNHCH₂CH₂Cl |
| F-18 | —OCH₂CH₂NO₂ |
| F-19 | —OCH₂CH₂SO₃H |
| F-20 | —OCH₂CH₂CH₂SO₃H |
| F-21 | —OCH₂CH₂CH₂CH₂SO₃H |
| F-22 | —OCH₂CH₂NHSO₂N(CH₃)₂ |
| F-23 | 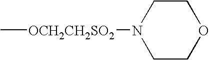 |
| F-24 | —OCH₂CH₂OCH₃ |
| F-25 | —OCH₂CH₂SCH₃ |

TABLE 10

| No. | Group |
|---|---|
| F-26 | —OCH₂CH₂SOCH₃ |
| F-27 | —OCH₂CH₂SO₂CH₃ |
| F-28 | —OCH₂CN |
| F-29 | —OCH₂C(═NH)NH₂ |
| F-30 | —OCH₂CSNH₂ |
| F-31 | —OCH₂COCH₃ |
| F-32 | —OCH₂COCF₃ |
| F-33 | —OCH₂CHO |
| F-34 | —OCH₂CH═NOCH₂C≡CH |
| F-35 | —OCH₂CH═NN(CH₃)₂ |
| F-36 | —OCH₂COOH |
| F-37 | —OCH₂COOCH₃ |
| F-38 | —OCH₂COOCH₂CH₂Cl |
| F-39 | —OCH₂COOCH₂CH═CH₂ |
| F-40 | —OCH₂COOCH₂C≡CH |
| F-41 | 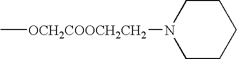 |
| F-42 | 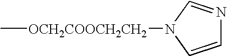 |
| F-43 | —OCH₂COOCH₂CH₂OCH₃ |
| F-44 | —OCH₂COOCH₂CH₂SCH₃ |
| F-45 | —OCH₂COOCH₂CH₂SOCH₃ |
| F-46 | —OCH₂COOCH₂CH₂SO₂CH₃ |
| F-47 | —OCH₂COOCH₂CH₂OH |
| F-48 | —OCH₂COO(CH₂)₉OH |
| F-49 | —OCH₂COOCH₂CH₂OSO₂N(CH₃)₂ |
| F-50 | —OCH₂COOCH₂CH₂COCH₃ |

TABLE 11

| No. | Group |
|---|---|
| F-51 | —OCH₂COOCH₂CH₂ON(CH₃)₂ |
| F-52 | —OCH₂COOCH₂CH₂N(CH₃)₂ |
| F-53 | —OCH₂COOCH₂CH₂N(OC₂H₅)C₂H₅ |
| F-54 | —OCH₂COOCH₂CH₂NHCOCH₃ |
| F-55 | —OCH₂COOCH₂CH₂N(CH₃)COCH₃ |
| F-56 | —OCH₂COOCH₂CH₂NHCOOCH₂CH₂OCH₃ |
| F-57 | —OCH₂COOCH₂CH₂NHCOSCH₂CH═CH₂ |
| F-58 | —OCH₂COOCH₂CH₂NHCONHC₂H₅ |
| F-59 | —OCH₂COOCH₂CH₂NHCON(CH₃)₂ |
| F-60 | —OCH₂COOCH₂CH₂NHCON(OCH₃)CH₃ |
| F-61 | —OCH₂COOCH₂CH₂NHCSNHCH₂CH₂Cl |
| F-62 | —OCH₂COOCH₂CH₂NHSO₂N(CH₃)₂ |
| F-63 | —OCH₂COOCH₂CH₂CN |
| F-64 | —OCH₂COOCH₂CH₂NO₂ |
| F-65 | —OCH₂COOCH₂CH₂SO₃H |
| F-66 | 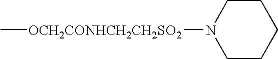 |
| F-67 | —OCH₂CONHCH₂CH₂SO₂N(CH₃)₂ |
| F-68 | —OCH₂COSCH₃ |
| F-69 | —OCH₂CONH₂ |
| F-70 | —OCH₂CONHCH₃ |
| F-71 | —OCH₂CON(CH₃)₂ |
| F-72 | —OCH₂CON(CH₃)CH₂C≡CH |
| F-73 | —OCH₂CON(OCH₃)CH₃ |
| F-74 | —OCH₂CONHOCH₃ |
| F-75 | —OCH₂CONHOCH₂CH═CH₂ |

TABLE 12

| No. | Group |
|---|---|
| F-76 | —OCH₂COOCH₂COOCH₃ |
| F-77 | —OCH₂COSCH₂COOCH₃ |
| F-78 | —OCH₂CONHCH₂COOCH₃ |
| F-79 | —OCH₂CONHCH₂CON(CH₃)₂ |
| F-80 | —OCH₂CONHCH₂CN |
| F-81 | —OCH₂CONHCH₂C(═NH)NH₂ |
| F-82 | —OCH₂CONHSO₂CH₃ |
| F-83 | 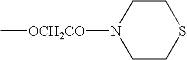 |
| F-84 | —OCH₂CONHN(CH₃)₂ |
| F-85 | —OCH₂CONHNHCOOC₂H₅ |
| F-86 | —OCH₂CONHNHCSNH(c)C₆H₁₁ |
| F-87 | —SCH₂CN |
| F-88 | —CH₂SCH₂COOCH₃ |
| F-89 | —CH₂SOCH₂COOCH₃ |
| F-90 | —CH₂SO₂CH₂COOCH₃ |
| F-91 | —NHCH₂COOCH₃ |
| F-92 | —NHCH₂CH₂N(CH₃)₂ |
| F-93 | —N(COCH₃)CH₂CH₂OH |
| F-94 | —CH₂OCH₂COOCH₃ |
| F-95 | —CH₂SCH₂COOH |
| F-96 | —CH₂SOCH₂COOH |
| F-97 | —CH₂SO₂CH₂COOH |

Examples of groups belonging to the G₀ group and the G group are shown in Table 13 to Table 16.

TABLE 13

| No. | Group |
|---|---|
| G-1 | 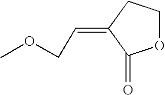 |
| G-2 | 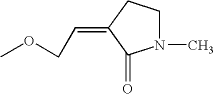 |
| G-3 | 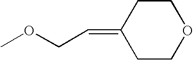 |
| G-4 | 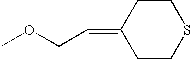 |
| G-5 | 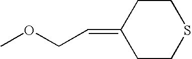 |
| G-6 | 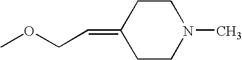 |
| G-7 | —OCH₂CH═CH₂ |
| G-8 | —OCH₂C≡CH |
| G-9 | —OCH₂CH═CHCl |
| G-10 | —SCH═CHOCH₃ |
| G-11 | —SO₂CH═CHOCH₃ |
| G-12 | —OCH═CHCOCH₃ |
| G-13 | —OCH═CHCHO |
| G-14 | —OCH═CHCH═NCH₂CH═CH₂ |
| G-15 | —OCH═CHCH═NOCH₃ |
| G-16 | —OCH═CHCH═NN(CH₃)₂ |
| G-17 | —OCH═CHCN |
| G-18 | —OCH═CHC(═NH)NH₂ |
| G-19 | —OCH═CHCOOH |
| G-20 | —OCH₂C≡CCOOH |

TABLE 14

| No. | Group |
|---|---|
| G-21 | —OCH═CHCOOCH₃ |
| G-22 | —OCH═CHCOOCH₂CH₂Cl |
| G-23 | —OCH═CHCOOCH₂CH═CH₂ |
| G-24 | —OCH═CHCOOCH₂C≡CH |
| G-25 | 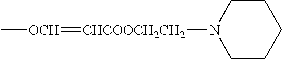 |
| G-26 | 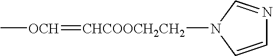 |
| G-27 | —OCH═CHCOOCH₂CH₂OCH₃ |
| G-28 | —OCH═CHCOOCH₂CH₂SCH₃ |
| G-29 | —OCH═CHCOOCH₂CH₂SOCH₃ |
| G-30 | —OCH═CHCOOCH₂CH₂SO₂CH₃ |
| G-31 | —OCH═CHCOOCH₂CH₂OH |
| G-32 | —OCH═CHCOOCH₂CH₂OSO₂N(CH₃)₂ |
| G-33 | —OCH═CHCOOCH₂CH₂COCH₃ |
| G-34 | —OCH═CHCOOCH₂CH₂ON(CH₃)₂ |
| G-35 | —OCH═CHCOOCH₂CH₂N(CH₃)₂ |
| G-36 | —OCH═CHCOOCH₂CH₂N(OC₂H₅)C₂H₅ |
| G-37 | —OCH═CHCOOCH₂CH₂NHCOCH₃ |
| G-38 | —OCH═CHCOOCH₂CH₂N(CH₃)COCH₃ |
| G-39 | —OCH═CHCOOCH₂CH₂NHCOOCH₂CH₂OCH₃ |
| G-40 | —OCH═CHCOOCH₂CH₂NHCOSCH₂CH═CH₂ |
| G-41 | —OCH═CHCOOCH₂CH₂NHCONHC₂H₅ |
| G-42 | —OCH═CHCOOCH₂CH₂NHCON(CH₃)₂ |
| G-43 | —OCH═CHCOOCH₂CH₂NHCON(OCH₃)CH₃ |
| G-44 | —OCH═CHCOOCH₂CH₂NHCSNHCH₂CH₂Cl |
| G-45 | —OCH═CHCOOCH₂CH₂NHSO₂N(CH₃)₂ |
| G-46 | —OCH═CHCOOCH₂CH₂C(═NH)NH₂ |
| G-47 | —OCH═CHCOOCH₂CH₂NO₂ |
| G-48 | —OCH═CHCOOCH₂CH₂SO₃H |

TABLE 15

| No. | Group |
|---|---|
| G-49 | 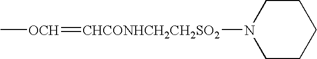 |
| G-50 | —OCH═CHCONHCH₂CH₂SO₂N(CH₃)₂ |
| G-51 | —OCH═CHCOSCH₃ |
| G-52 | —OCH═CHCON(CH₃)CH₂C≡CH |
| G-53 | —OCH═CHCON(OCH₃)CH₃ |
| G-54 | —OCH═CHCONHOCH₃ |
| G-55 | —OCH═CHCONHOCH₂CH═CH₂ |
| G-56 | —OCH═CHCONHCH₂COOCH₃ |
| G-57 | —OCH═CHCONHCH₂CON(CH₃)₂ |
| G-58 | —OCH═CHCONHSO₂CH₃ |
| G-59 | 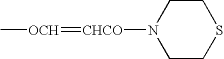 |
| G-60 | —OCH═CHCONHN(CH₃)₂ |
| G-61 | —OCH═CHCONHNHCOOC₂H₅ |
| G-62 | —OCH═CHCONHNHCSNH(c)C₆H₁₁ |
| G-63 | 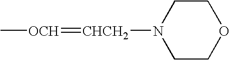 |
| G-64 | 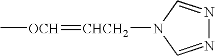 |
| G-65 | —OCH═CHCH₂OCH₃ |
| G-66 | —OCH═CHCH₂SCH₃ |
| G-67 | —OCH═CHCH₂SOCH₃ |
| G-68 | —OCH═CHCH₂SO₂CH₃ |
| G-69 | —OCH═CHCH₂OH |
| G-70 | —OCH═CHCH₂OCOCH₃ |

TABLE 16

| No. | Group |
|---|---|
| G-71 | —OCH₂C≡CCH₂OH |
| G-72 | —OCH═CHCH₂ON(CH₃)₂ |
| G-73 | —OCH═CHCH₂N(CH₃)₂ |
| G-74 | —OCH═CHCH₂N(CH₂CH═CH₂)₂ |
| G-75 | —OCH═CHCH₂N(OH)CH₃ |
| G-76 | —OCH═CHCH₂NO₂ |
| G-77 | —OCH═CHCH₂SO₃H |
| G-78 | —SCH₂CH═CH₂ |
| G-79 | —SOCH₂CH═CH₂ |
| G-80 | —SO₂CH₂CH═CH₂ |
| G-81 | —SCH═CHCOOH |
| G-82 | —CH₂NHCH═CHCOOH |
| G-83 | —CH₂OCH₂CH═CH₂ |
| G-84 | —CH₂OCH═CHCOOH |

Examples of groups belonging to the H₀ group and the H group are shown in Table 17.

TABLE 17

| No. | Group |
|---|---|
| H-1 | —CH₂NHCN |
| H-2 | —N(COCH₃)CN |
| H-3 | —NHC(═NH)NHOH |
| H-4 | —NHC(═NH)N(CH₂CH═CH₂)CH₃ |
| H-5 | —C(═NH)NHCH₂CH═CH₂ |
| H-6 | —N═CHN(CH₃)₂ |
| H-7 | —N(CH₃)C(CH₃)═NOCH₂C≡CH |
| H-8 | —NHCONHCOCH₃ |
| H-9 | —NHCONHSO₂CH₃ |
| H-10 | —NHCOCN |
| H-11 | —NHCOCOOCH₃ |
| H-12 | —NHCOCOOH |

Examples of groups belonging to the I₀ group and the I group are shown in Table 18.

TABLE 18

| No. | Group |
|---|---|
| I-1 | —NHCOCH═CH₂ |
| I-2 | —NHCSCH═CH₂ |
| I-3 | —NHCOCF═CH₂ |
| I-4 | —NHCOC≡CH |
| I-5 | —NHCOCH₂OCH₃ |
| I-6 | —NHCOCH₂SCH₃ |
| I-7 | —NHCOCH₂COCH₃ |
| I-8 | —NHCOCH₂OH |
| I-9 | —NHCOCH₂ONH₂ |
| I-10 | —NHCOCH₂N(CH₃)CH₂C≡CH |
| I-11 | —NHCOCH₂NHCOCH₃ |
| I-12 | —NHCOCH₂COOCH₃ |
| I-13 | —NHCOCH₂CN |
| I-14 | —NHCOCH₂NO₂ |
| I-15 | —NHCOCH₂SO₃H |
| I-16 | —NHCOCH₂SO₂N(CH₃)₂ |
| I-17 | —NHCSCH₃ |
| I-18 | —NHCSCH₂N(CH₃)₂ |
| I-19 | —NHCOOCH₂CH₂OCH₃ |
| I-20 | —NHCOOCH₂CN |
| I-21 | —NHCOOCH₂CH₂NO₂ |
| I-22 | —NHCOOCH₂CH₂NHCOCH₃ |
| I-23 | —NH(CS)OCH₃ |
| I-24 | —NH(CO)SCH₃ |
| I-24 | —NHCONHCH₂CH₂OCH₃ |
| I-25 | —NHCSNHCH₃ |
| I-26 | —NHSO₂CH═CH₂ |
| I-27 | —NHSO₂CH₂CH═CH₂ |
| I-28 | —NHSO₂CH₂C≡CH |
| I-29 | —NHSO₂CH₂COCH₃ |
| I-30 | —NHSO₂CH₂CN |
| I-31 | —NHSO₂CH₂NO₂ |
| I-32 | —NHSO₂CH₂COOH |
| I-33 | —NHSO₂CH₂COOCH₃ |

Examples of groups belonging to the J₀ group and the J group are shown in Table 19.

TABLE 19

| No. | Group |
|---|---|
| J-1 | —COCH═CH₂ |
| J-2 | —COC≡CH |
| J-3 | —COC≡CCF₃ |
| J-4 | —COCH₂SCH₃ |
| J-5 | —COCH₂OH |
| J-6 | —COCH₂N(CH₃)₂ |
| J-7 | —CSCH₃ |
| J-8 | —CSCF₃ |
| J-9 | —CH═NCH₃ |
| J-10 | —CH═NOCH₃ |
| J-11 | —COCN |
| J-12 | —COC(═NH)NH₂ |
| J-13 | —COCOOCH₃ |
| J-14 | —CH₂OCON(CH₃)₂ |

Examples of groups belonging to the K₀ group and the K group are shown in Table 20.

TABLE 20

| No. | Group |
|---|---|
| K-1 | —CONHSO₂CH₃ |
| K-2 | —CONHOH |
| K-3 | —CONHOCH₃ |
| K-4 | —CONHOCH₂CH═CH₂ |
| K-5 | —CONHCH₂CH₂OH |
| K-6 | —CONHCH₂CH₂OCH₃ |
| K-7 | —CONHCH₂OCH₃ |
| K-8 | —CONHCH₂CH═CH₂ |
| K-9 | —CONHCH₂C≡CH |
| K-10 | —CONHCH₂CN |
| K-11 | —CONHCH₂COOH |
| K-12 | —CONHCH₂COOCH₃ |
| K-13 | —CONHCH₂CONH₂ |
| K-14 | —CONHCH₂CONHCH₃ |
| K-15 | —CONHCH₂CONH(CH₃)₂ |
| K-16 | —CONHCH(CH₂COOH)COOH |
| K-17 | —CONHCH(CH₂COOCH₃)COOCH₃ |
| K-18 | —CONHCH₂CH₂N(CH₃)₂ |

Examples of groups belonging to the L₀ group and the L group are shown in Table 21.

TABLE 21

| No. | Group |
|---|---|
| L-1 | —SO₂NHOH |
| L-2 | —SO₂NHOCH₃ |
| L-3 | —SO₂NHOCH₂CH═CH₂ |
| L-4 | —SO₂NHCH₂CH₂OCH₃ |
| L-5 | —SO₂NHCH₂CH═CH₂ |
| L-6 | —SO₂NHCH₂C≡CH |
| L-7 | —SO₂NHCH₂CN |
| L-8 | —SO₂NHCOCH₃ |
| L-9 | —SO₂NHCH₂COOH |
| L-10 | —SO₂NHCH₂COOCH₃ |
| L-11 | —SO₂NHCH₂CONH₂ |
| L-12 | —SO₂NHCH₂CONHCH₃ |
| L-13 | —SO₂NHCH₂CON(CH₃)₂ |
| L-14 | —SO₂NHCH(CH₂COOH)COOH |
| L-15 | —NHSO₂N(CH₃)₂ |

Examples of groups belonging to the M₀ group and the M group are shown in Table 22.

TABLE 22

| No. | Group |
|---|---|
| M-1 | —N═C(—SCH₃)CH₃ |
| M-2 | —N═C(—OCH₃)OCH₃ |
| M-3 | —N═C(—SCH₃)OCH₃ |
| M-4 | —N═C(—SCH₃)SCH₃ |
| M-5 | —N═C(—SCH₃)NHCH₃ |
| M-6 | —N(CH₃)C(—SCH₃)═NCH₃ |
| M-7 | —N(CH₃)OCH₂CH═CH₂ |
| M-8 | —N(CH₂CH═CH₂)OCH₂CH═CH₂ |

Examples of groups belonging to the N₀ group and the N group are shown in Table 23.

TABLE 23

| No. | Group |
|---|---|
| N-1 | —CH₂P(═O)(OH)₂ |
| N-2 | —CH₂P(═O)(OCH₃)₂ |
| N-3 | —CH₂P(═O)(OCH₃)—CH₃ |
| N-4 | —CH₂P(═O)(OCH₃)—CH(OH)CH₃ |
| N-5 | —CH₂P(═O)(OCH₃)—CH₂CH₂OH |
| N-6 | —CH₂P(═O)(OCH₃)—CH₂COOCH₃ |

Examples of the aforementioned groups belonging to the X₀ group to the Z₀ group and the X group to the Z group are shown in the following Table 24 to Table 26. When said groups have geometrical isomers, all of the geometrical isomers are included, and when said groups have tautomers, all of the tautomers are included.

Examples of groups belonging to the X₀ group and the X group are shown in Table 24.

TABLE 24

| No. | Group |
|---|---|
| X-1 | —CH₃ |
| X-2 | —C₂H₅ |
| X-3 | —CF₃ |
| X-4 | —CH═CHCH₃ |
| X-5 | —CH₂CH═CH₂ |
| X-6 | —C≡CH |
| X-7 | —F |
| X-8 | —Cl |
| X-9 | —Br |
| X-10 | —NO₂ |
| X-11 | —CN |
| X-12 | —OCH₃ |
| X-13 | —SCH₃ |
| X-14 | —SOC₄H₉ |
| X-15 | —SO₂C₄H₉ |
| X-16 | —OCHF₂ |
| X-17 | —OCF₃ |
| X-18 | —OCF₂CHF₂ |
| X-19 | —SCF₃ |
| X-20 | —CH₂OCH₃ |
| X-21 | —COCH₃ |
| X-22 | —OCOCH₃ |
| X-23 | —COOH |
| X-24 | —COOCH₃ |
| X-25 | —CH═CHCOOH |
| X-26 | —N(CH₃)₂ |
| X-27 | —NHCOCH₃ |
| X-28 | —NHCOOCH₃ |
| X-29 | —CONH₂ |
| X-30 | —CON(CH₃)₂ |
| X-31 | —NHCON(CH₃)₂ |
| X-32 | —NHC(═NH)NH₂ |
| X-33 | —NHSO₂CF₃ |
| X-34 | —SO₂N(CH₃)₂ |

Examples of groups belonging to the Y₀ group and the Y group are shown in Table 25.

TABLE 25

| No. | Group |
|---|---|
| Y-1 | 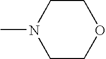 |
| Y-2 | 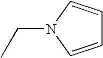 |
| Y-3 | 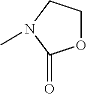 |
| Y-4 | 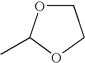 |
| Y-5 | 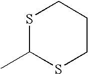 |
| Y-6 | 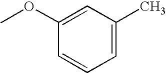 |
| Y-7 | 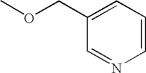 |
| Y-8 | 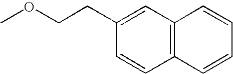 |
| Y-9 | 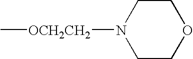 |
| Y-10 | 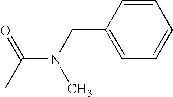 |

Examples of the α ring, the A0 ring or the A ring fused to the Z₀ group or the Z group are shown in Table 26.

TABLE 26

| | α ring, A0 |
|---|---|
| | ring or A ring |
| | fused to Z₀ |
| No. | group or Z group |
| Z-1 | 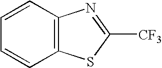 |
| Z-2 | 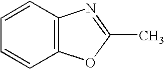 |
| Z-3 | 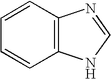 |
| Z-4 | 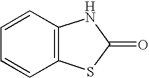 |
| Z-5 | 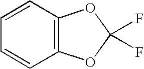 |
| Z-6 | 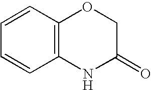 |
| Z-7 | 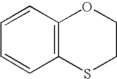 |
| Z-8 | 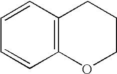 |
| Z-9 | 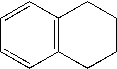 |
| Z-10 | 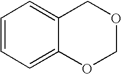 |

Examples of Q_{A0} and Q_{A} are shown in Table 27 to Table 28.

TABLE 27

| No. | Group |
|---|---|
| Q-1 | —OH |
| Q-2 | 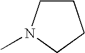 |
| Q-3 | 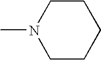 |
| Q-4 | 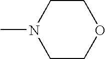 |
| Q-5 | —OCOCH₃ |
| Q-6 | —OSO₂N(CH₃)₂ |
| Q-7 | —NHCH₂CH═CH₂ |
| Q-8 | —NHCH₂C≡CH |
| Q-9 | —NHCH₂CH₂OCH₃ |
| Q-10 | —OCH₃ |
| Q-11 | —OCH₂CH₂(c)C₆H₁₁ |
| Q-12 | —OCH₂CH═CH₂ |
| Q-13 | —OCH₂C≡CH |
| Q-14 | —OCH₂COOH |
| Q-15 | —OCH₂COOCH₃ |
| Q-16 | —OCH₂CONH₂ |
| Q-17 | —OCH₂CN |
| Q-18 | —OCH₂CH₂OH |
| Q-19 | —OCH₂CH₂OCH₃ |
| Q-20 | —OCH₂CH₂N(CH₃)₂ |

TABLE 28

| No. | Group |
|---|---|
| Q-21 | —OCH₂COCH₃ |
| Q-22 | —OCOC₆H₅ |
| Q-23 | —OCH₂C₆H₅ |
| Q-24 | 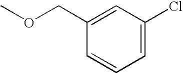 |
| Q-25 | 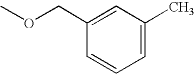 |
| Q-26 | 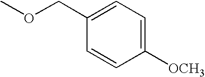 |

Examples of T_{A0} and T_{A} are shown in Table 29.

TABLE 29

| No. | Group |
|---|---|
| T-1 | —H |
| T-2 | —CH₃ |
| T-3 | —CH₂CH₂(c)C₆H₁₁ |
| T-4 | —CH₂CH═CH₂ |
| T-5 | —CH₂C≡CH |
| T-6 | —CH₂C₆H₅ |
| T-7 | —CH₂COOH |
| T-8 | —CH₂COOCH₃ |
| T-9 | —CH₂CONH₂ |
| T-10 | —CH₂CN |
| T-11 | —CH₂CH₂OH |
| T-12 | —CH₂CH₂OCH₃ |
| T-13 | —CH₂CH₂N(CH₃)₂ |
| T-14 | —CH₂COCH₃ |
| T-15 | —CH₂CF₃ |
| T-16 | —Ph |
| T-17 | 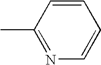 |

An example of the compound (II) includes a cinnamoyl compound represented by the formula (II″):

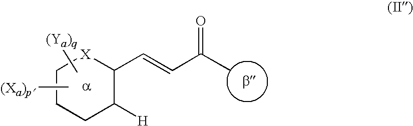

wherein α, X_{α}, Y_{α} and q are as defined above, x represent a methine group or a nitrogen atom, p′ represents 1, 2 or 3, and when p′ is not less than 2, X_{α}s are same or different, and the sum of p′ and q is not more than 3, and β″ represents a group represented by formula (II″-1):

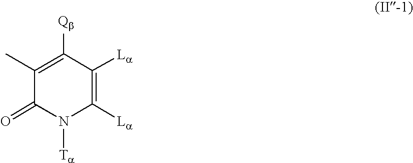

(wherein Q_{β}, T_{α}, K_{α} and L_{α} are as defined above), a group represented by the formula (II″-7):

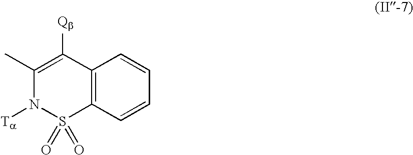

(wherein Q_{β} and T_{α} are as define above), or a group represented by the formula (II″-8):

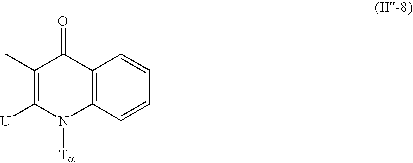

(wherein U and T_{α} are as defined above) In the cinnamoyl compound (II″), when x is a methine group, the methine group has no substituent. In a specific example of the cinnamoyl compound (II″), β″ is a group represented by the formula (II″-1), (II″-7) or (II″-8), wherein the group (II″-1) is a group represented by the formula (II′″-1):

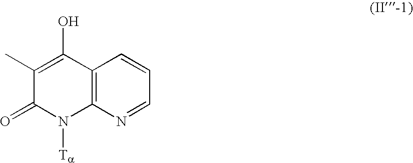

(wherein T_{α} is as defined above) and T_{α} is a hydrogen atom or a C1-C10 alkyl group, or Q_{β} of the group (II″-7) is a hydroxyl group and T_{α} of the group (II″-7) is a C1-C10 alkyl group, or T_{α} of the group (II″-8) is a hydrogen atom or a C1-C10 alkyl group and U of the group (II″-8) is a hydrogen atom. In a more specific example of the cinnamoyl group (II″), β″ is a group represented by the formula (II′″-1) and T_{α} is a hydrogen atom or a C1-C10 alkyl group.

An example of the compound (III) includes a cinnamoyl compound represented by the formula (III′):

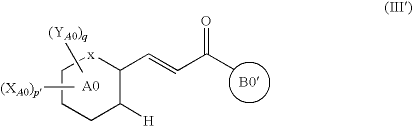

wherein A0, X_{A0}, Y_{A0}, p′, q and x are as defined above, and when p′ is not less than 2, X_{A0}s are the same or different, and the sum of p′ and q is not more than 3, and B0′ represents a group represented by the formula (III′-1):

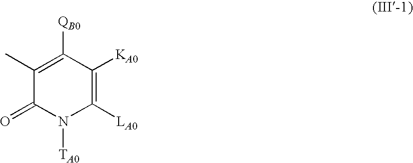

(wherein Q_{B0}, T_{A0}, K_{A0} and L_{A0} are as defined above) or a group represented by the formula (III′-7):

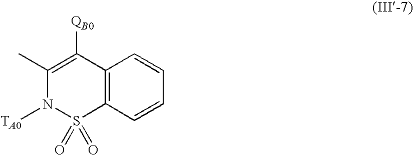

(wherein Q_{B0} and T_{A0} are as defined above) or a group represented by the formula (III′-8):

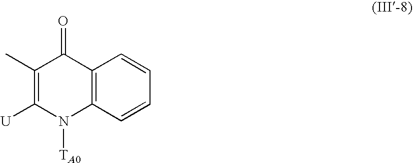

(wherein U and T_{A0} are as defined above). In the cinnamoyl compound (III′), when x is a methine group, the methine group has no substituent. In a specific example of the cinnamoyl compound (III′), B0′ is a group represented by the formula (III′-1), (III′-7) or (III′-8), wherein the group (III′-1) is a group represented by the formula (III″-1):

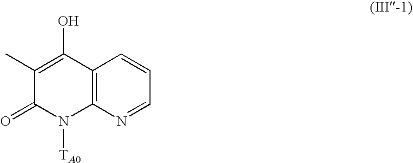

(wherein T_{A0} is as defined above) and T_{A0} is a hydrogen atom or C1-C10 alkyl group, or Q_{B0} of the group (III′-7) is a hydroxyl group and T_{A0} of the group (III′-7) is a C1-C10 alkyl group, or T_{A0} of the group (III′-8) is a hydrogen atom or a C1-C10 alkyl group and U of the group (III′-8) is a hydrogen atom. In a more specific example of the cinnamoyl compound (III′), B0′ is the group (III″-1) and T_{A0} is a hydrogen atom or a C1-C10 alkyl group.

An example of the compound (IV) includes a cinnamoyl compound represented by the formula (IV′):

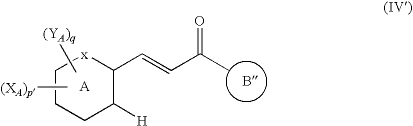

wherein A, X_{A}, Y_{A}, p′, q and x are as defined above, and when p′ is not less than 2, X_{A}s are the same or different, and the sum of p′ and q is not more than 3, and B′ represents a group represented by the formula (IV′-1):

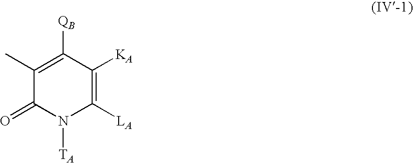

(wherein Q_{B}, T_{A}, K_{A} and L_{A} are as defined above) or a group represented by the formula (IV′-7):

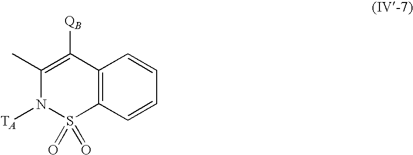

(wherein Q_{B} and T_{A} are as define above) or a group represented by the formula (IV′-8):

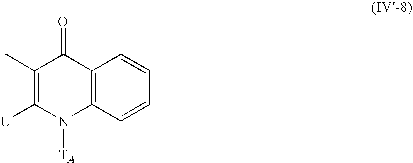

(wherein U and T_{A} are as defined above). In the cinnamoyl compound (IV′), when x is a methine group, the methine group has no substituent. In a specific example of the cinnamoyl compound (IV′), B′ is a group represented by the formula (IV′-1), (IV′-7) or (IV′-8), wherein the group (IV′-1) is a group represented by the formula (IV″-1):

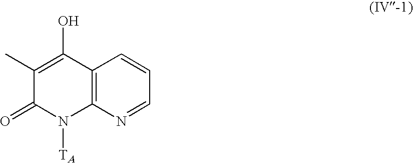

(wherein T_{A} is as defined above) and T_{A} is a hydrogen atom or a C1-C10 alkyl group, or Q_{B} of the group (IV′-7) is a hydroxyl group and T_{A} of the group (IV′-7) is a C1-C10 alkyl group, or T_{A} of the group (IV′-8) is a hydrogen atom or a C1-C10 alkyl group and U of the group (IV′-8) is a hydrogen atom. In a more specific example of the cinnamoyl compound (IV′), B′ is the group (IV″-1) and T_{A} is a hydrogen atom or a C1-C10 alkyl group.

An example of the compounds (V) includes a cinnamoyl compound represented by the formula (V″):

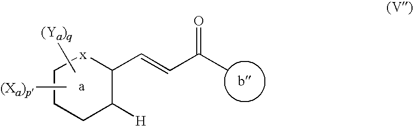

wherein a, Xₐ, Yₐ, p′, q and x are as defined above, and when p′ is not less than 2, Xₐs are the same or different, and the sum of p′ and q is not more than 3, and b″ represents a group represented by the formula (V″-1):

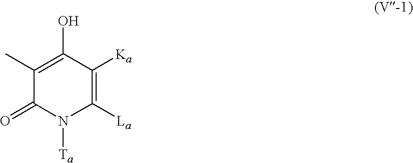

(wherein Kₐ, Lₐ and Tₐ are as defined above) or a group represented by the formula (V″-7):

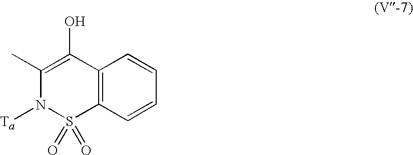

(wherein Tₐ is as defined above) or a group represented by the formula (V″-8):

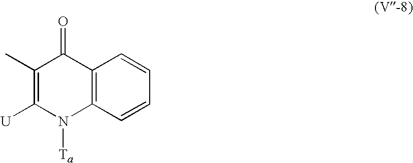

(wherein U and Tₐ are defined above). In the cinnamoyl compound (V″), when x is a methine group, the methine group has no substituent. In a specific example of the cinnamoyl compound (V″), b″ is a group represented by the formula (V″-1), (V″-7) or (V″-8), wherein the group (V″-1) is a group represented by the formula (V′″-1):

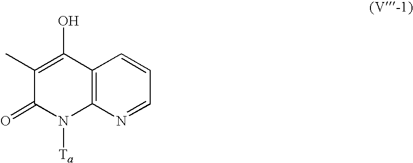

(wherein Tₐ is as defined above) and Ta is a hydrogen atom or a C1-C10 alkyl group, or Tₐ of the group (V″-7) is a C1-C10 alkyl group, or Tₐ of the group (V″-8) is a hydrogen atom or C1-C10 alkyl group and U of the group (V″-8) is hydrogen atom. In a more specific example of the cinnamoyl compound (V″), b″ is the group (V′″-1) and T_{A} is a hydrogen atom or a C1-C10 alkyl group.

A further specific example of the compound (V) includes a cinnamoyl compound represented by the formula (V′″):

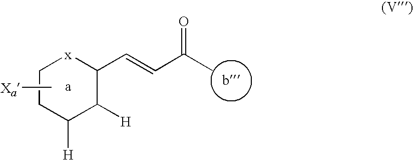

wherein a and x are as defined above, Xₐ′ represents a CH₃OCH₂CONH— group, a CH₃OCOCH₂NHCO— group, a HOCOCH₂NHCO— group, a CH₃OCH₂CH₂NHCO— group, a CH₃OCOCH₂O— group, a HOCOCH₂O— group, a HOCH₂CH₂O— group, a CH₃OCH₂CH₂OCONH— group or a (CH₃)₂NCH₂CH₂O— group, and b′″ represents a group represented by the formula (V″″-1):

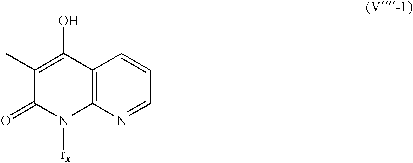

(wherein rₓ represents a hydrogen atom or a methyl group) or a group represented by the formula (V′″-7):

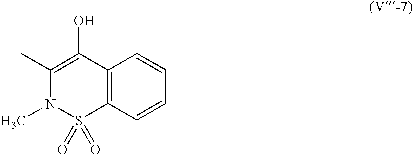

or a group represented by the formula (V′″-8):

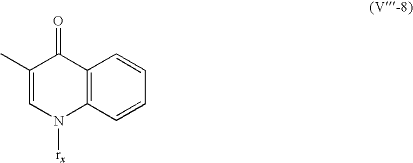

(wherein rₓ is as defined above). In the cinnamoyl compound (V′″), when x is a methine group, the methine group has no substituent. In a still more specific example of the cinnamoyl compound (V′″), b′″ is a group represented by the formula (V″″-1).

Although WO 97/35565, JP09227547, WO 00/20371, JP2002371078, WO 01/79187 and WO 92/18483 disclose compounds having a certain conceptional skeleton, they do not describe the effect of suppressing transcription of an extracellular matrix gene in a tissue, consequently, the effect of suppressing the accumulation of an extracellular matrix.

The compound (I) or (II) can be produced, for example, by reacting a compound represented by the formula (α) (wherein α, X_{α}, Y_{α}, p and q are as defined above) and a compound represented by the formula (β) (wherein β is as defined above).

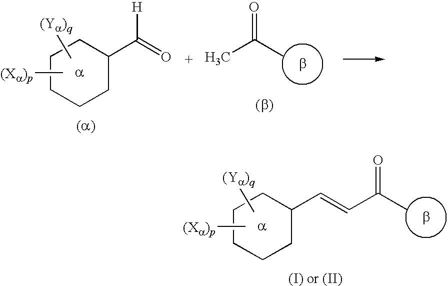

In the compound (β), when β is a group represented by the above formula (I-2), (I-3), (I-6) or (I-8), the compound (I) or (II) in which β is the group (I-2), (I-3), (I-6) or (I-8) can be produced, for example, according to the references (Liebigs Annalen der Chemie, (1979), D(6), 769, J. Heterocyclic Chem. (1981), 18(3), 603, Polish J. Chem. (1982), 56(2), 419, Synthetic Communication (1986), 16(10), 1195, Ukrainskii Khimicheskii Zhurnal (1982), 48(7), 772).

In the compound (β), when β is a group represented by the above formula (I-1), (I-4), (I-5) or (I-7), the compound (I) or (II) can be produced by reacting the compound (α) and the compound (β) in which β is the above group (I-1), (I-4), (I-5) or (I-7). In the reaction, the reaction temperature is usually from room temperature to the reflux temperature of a solvent, and the reaction time is usually from instant to about 24 hours. The reaction is usually performed in the presence of a base. Examples of the base used include organic basis such as piperidine, pyridine, triethylamine, N,N-dimethylaniline, tributylamine, morpholine, N-methylmorpholine and the like, and inorganic bases such as sodium hydroxide, potassium hydroxide, potassium carbonate and the like. In the reaction, usually, 0.5 to 2 mol of the compound (β) and 0.1 to 1.0 mol of the base are used per 1 mol of the compound (α). In the reaction, a solvent is not necessarily required, but the reaction is usually performed in the presence of a solvent. Examples of a solvent useable in the reaction include lower alcohols such as methanol, ethanol and the like, aliphatic hydrocarbons such as hexane, petroleum ether and the like, aromatic hydrocarbons such as benzene, toluene and the like, halogenated hydrocarbons such as chloroform, dichloroethane and the like, ethers such as dietyl ether, dioxane, tetrahydrofuran and the like, ketones such as acetone, methyl ethyl ketone and the like, esters such as ethyl acetate, diethyl carbonate and the like, nitrites such as acetonitrile, isobutyronitrile and the like, amides such as formamide, N,N-dimethylformamide and the like, sulfur compounds such as dimethyl sulfoxide and the like, and their mixtures. After completion of the reaction, a reaction solution is extracted with an organic solvent, washed with water, and subjected to a conventional post-treatment such as concentration of an organic layer under reduced pressure or the like, and then, if necessary, purified by chromatography, recrystallization or the like to obtain the compound (β).

The compound (III) can be produced, for example, by reacting a compound represented by the formula (A0) (wherein A, X_{A0}, Y_{A0}, p and q are as defined above) and a compound represented by the formula (B0) (wherein B0 is as defined above).

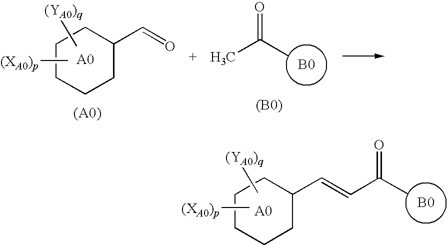

The compound (IV) can be produced, for example, by reacting a compound represented by the formula (A) (wherein A, X_{A}, Y_{A}, p and q are as defined above) and a compound represented by the formula (B) (wherein B is as defined above).

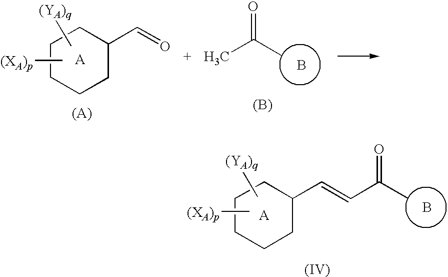

The compound (V) can be produced, for example, by reacting a compound represented by the formula (a) (wherein a, Xₐ, Yₐ, p and q are as defined above) and a compound represented by the formula (b) (wherein b is as defined above.

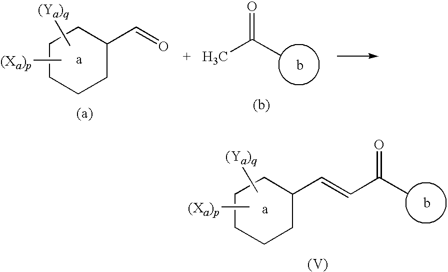

A part of compounds represented by the formula (a) are described, for example, in EP330645, and are known, and can be produced by the method described in WO 2005/028439.

A part of compounds represented by the formula (b) are described, for example, in J. Organic Chem. (1965) 30, 2241, and are known. However the compound represented by the formula (VII-3) which is a part of compounds represented by the formula (VI-1) has never been reported, and is a novel substance.

The compound (VII-3) can be produced, for example, by subjecting the compound represented by the formula (VII-2) to a cyclization reaction. In the cyclization reaction, the reaction temperature is usually from room temperature to the reflux temperature of a solvent, and the reaction time is usually from instant to about 24 hours. The cyclization reaction is usually performed in the presence of a base, and examples of the base used include organic bases such as sodium methoxide, sodium ethoxide, n-butyllithium, pyridine, triethylamine, N,N-dimethylaniline, tributylamine, N-methylmorpholine and the like, and inorganic bases such as sodium hydride, sodium hydroxide, potassium hydroxide, potassium carbonate and the like. In the reaction, usually, 0.8 to 2 mol of the base is used per 1 mol of the compound (VII-2). In the reaction, a solvent is not necessarily required, but the reaction is usually performed in the presence of a solvent. Examples of a solvent useable in the reaction include lower alcohols such as methanol, ethanol and the like, aliphatic hydrocarbons such as hexane, petroleum ether and the like, aromatic hydrocarbons such as benzene, toluene and the like, halogenated hydrocarbons such as chloroform, dichloroethane and the like, ethers such as dietyl ether, dioxane, tetrahydrofuran and the like, ketones such as acetone, methyl ethyl ketone and the like, esters such as ethyl acetate, diethyl carbonate and the like, nitrites such as acetonitrile, isobutyronitrile and the like, amides such as formamide, N,N-dimethylformamide and the like, sulfur compounds such as dimethyl sulfoxide and the like, and their mixtures. After completion of the reaction, a reaction solution is extracted with an organic solvent, washed with water, and subjected to a conventional post-treatment such as concentration of an organic layer under reduced pressure or the like, and then, if necessary, purified by chromatography, recrystallization or the like to obtain the compound (VII-3).

The compound (VII-2) has never been reported, and is a novel substance. The compound (VII-2) can be produced by 3-oxobutyrylamidating the compound represented by the formula (VII-1). In the reaction, the reaction temperature is usually from room temperature to the reflux temperature of a solvent, and the reaction time is usually from instant to about 24 hours. The reaction is performed in the presence of a 3-oxobutyrylamidating agent, and examples of the 3-oxobutyrylamidating agent include diketene, 2,2,6-trimethyl-4H-1,3-dioxin-4-one, methyl acetoacetate, t-butyl acetoacetate and the like. In the reaction, usually, 0.8 to 7 mol of the 3-oxobutyrylamidating agent is used per 1 mol of the compound (VII-1). In the reaction, a solvent is not necessarily required, but the reaction is usually performed in the presence of a solvent. Examples of a solvent usable in the reaction include lower alcohols such as methanol, ethanol and the like, aliphatic hydrocarbons such as hexane, petroleum ether and the like, aromatic hydrocarbons such as benzene, toluene, xylene and the like, halogenated hydrocarbons such as chloroform, dichloroethane and the like, ethers such as diethyl ether, dioxane, tetrahydrofuran and the like, ketones such as acetone, methyl ethyl ketone and the like, esters such as ethyl acetate, diethyl carbonate and the like, nitrites such as acetonitrile, isobutyronitrile and the like, amides such as formamide, N,N-dimethylformamide and the like, sulfur compounds such as dimethyl sulfoxide and the like, and their mixtures. After completion of the reaction, a reaction solution is subjected to a post-treatment and purification in the same manner as in the case of the cyclization reaction of the compound (VII-2) to obtain the compound (VII-2).

The compound (VII-1) is described, for example, in Tetrahedron (1997), 53(47), 16061, and is known.

The compound represented by the formula (XI-2) which is a part of the compound (VII-3) can be produced by alkylating the compound represented by the formula (XI-1) which is a part of the compound (VII-3).

The reaction is performed by, for example, reacting the compound (XI-1) and an alkylating agent in the presence of a base.

The reaction of the compound (XI-1) and an alkylating agent in the presence of a base does not necessarily need a solvent, but is usually performed in a solvent. Examples of a solvent used in the reaction include acid amides such as N,N-dimethylformamide, N,N-dimethylacetamide and the like, sulfoxides such as dimethyl sulfoxide and the like, phosphoric acid amide compound such as hexamethylphosphoramide and the like, and ketones such as acetone, methyl ethyl ketone and the like.

Examples of a base used in the reaction include alkali metal hydrides such as sodium hydride, potassium hydride and the like, alkali metal carbonates such as potassium carbonate and the like, and silver oxide and the like.

Examples of an alkylating agent include alkylsulfonic acid esters such as methyl methanesulfonate and the like, arylsulfonic acid esters such as methyl p-toluenesulfonate, ethyl p-toluenesulfonate and the like, sulfuric acid esters such as dimethyl sulfate and the like, and halides such as methyl iodide, ethyl bromide and the like.

In the reaction, usually, 1 to 2 mol of the base and 1 to 2 mol of the alkylating agent are used per 1 mol of the compound (XI-1).

The reaction temperature is usually in a range of 0° C. to 100° C., and the reaction time is usually in a range of instant to 24 hours.

After completion of the reaction, a reaction solution is subjected to a post-treatment and purification in the same manner as in the case of the cyclization reaction of the compound (VII-2) to obtain the compound (XI-2).

Table 30 shows novel compounds (VII-3) represented by the compound numbers (1A-1) to (1A-8).

TABLE 30

| Compound No. | 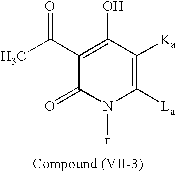 |
|---|---|
| (1A-1) | 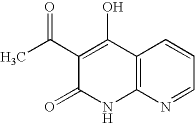 |
| (1A-2) | 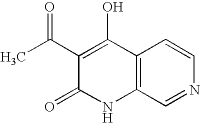 |
| (1A-3) | 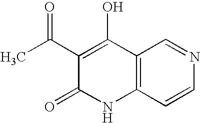 |
| (1A-4) | 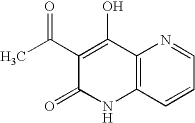 |
| (1A-5) | 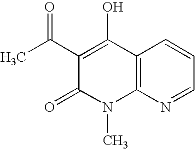 |
| (1A-6) | 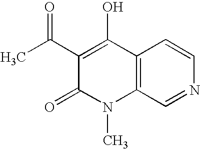 |
| (1A-7) | 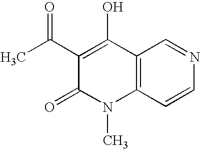 |
| (1A-8) | 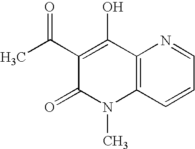 |

Table 31 shows novel compounds (VII-2) represented by the compound numbers (1B-1) to (1B-8).

TABLE 31

| Compound No. | 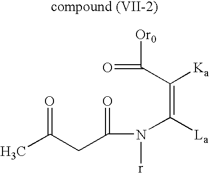 |
|---|---|
| (1B-1) | 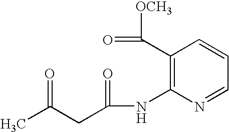 |
| (1B-2) | 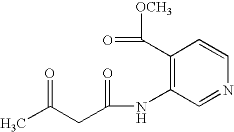 |
| (1B-3) | 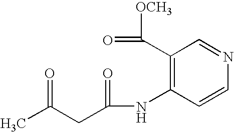 |
| (1B-4) | 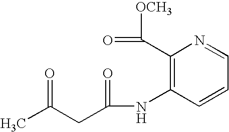 |
| (1B-5) | 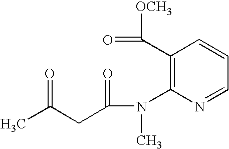 |
| (1B-6) | 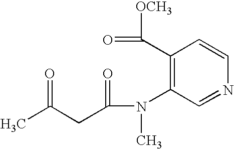 |
| (1B-7) | 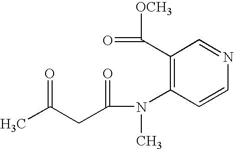 |
| (1B-8) | 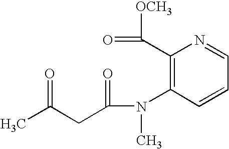 |

Among the compounds (V), examples of a compound represented by the formula (Va):

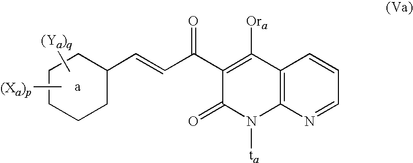

(wherein a, Xₐ, Yₐ, p, q, rₐ and tₐ are as defined above), which are represented by the compound numbers (1a) to (129a), are shown in Table 32 to Table 37.

### (Table 32 to Table 37)

In Table 32 to Table 37, in the compound numbers (1a) to (98a), (100a) to (104a) and (106a) to (123a), a represents a benzene ring.

TABLE 32

| Compound | | | |
|---|---|---|---|
| No. | Xₐ and Yₐ | rₐ | tₐ |
| (1a) | 3-CH═CHCN | —H | —H |
| (2a) | 3-OCH₂CH₂SCH₃ | —H | —H |
| (3a) | 3-OCH₂CH═CH₂ | —H | —H |
| (4a) | 2-OCH₂C≡CH | —H | —H |
| (5a) | 3-OCH₂C≡CH | —H | —H |
| (6a) | 4-OCH₂C≡CH | —H | —H |
| (7a) | 3-OCH₂COOCH₃ | —H | —H |
| (8a) | 3-OCH₃,4-OCH₂COOCH₃ | —H | —H |
| (9a) | 3-OCH₂COOH | —H | —H |
| (10a) | 3-OCH₂CN | —H | —H |
| (11a) | 3-OCH₂CN | —H | —CH₃ |
| (12a) | 3-OCH₂CN | —CH₃ | —CH₃ |
| (13a) | 4-OCH₂CN | —H | —H |
| (14a) | 3-CH₃,4-OCH₂CN | —H | —H |
| (15a) | 3-NO₂,4-OCH₂CN | —H | —H |
| (16a) | 3-F,4-OCH₂CN,5-OCH₃ | —H | —H |
| (17a) | 3-NHCOCH═CH₂ | —H | —H |
| (18a) | 3-NHCOCH₂OCH₃ | —H | —H |
| (19a) | 3-NHCOCH₂OCH₃ | —H | —CH₃ |
| (20a) | 4-NHCOCH₂OCH₃ | —H | —CH₃ |
| (21a) | 3-NHCOOCH₂CH₂OCH₃ | —H | —H |
| (22a) | 3-NHCONHCH₂CH₂OCH₃ | —H | —H |
| (23a) | 3-NHSO₂CH₂COOCH₃ | —H | —H |
| (24a) | 3-NHSO₂CH₂COOH | —H | —H |
| (25a) | 3-NHCOCH₂CN | —H | —H |
| (26a) | 3-CONHSO₂CH₃ | —H | —H |
| (27a) | 3-CONHCH₂CH₂OH | —H | —H |
| (28a) | 3-CONHCH₂COOCH₃ | —H | —H |
| (29a) | 3-CONHCH₂COOCH₃ | —H | —CH₃ |
| (30a) | 4-CONHCH₂COOCH₃ | —H | —H |

TABLE 33

| Compound | | | |
|---|---|---|---|
| No. | Xₐ and Yₐ | ra | tₐ |
| (31a) | 3-CONHCH₂CH₂OCH₃ | —H | —H |
| (32a) | 3-CONHCH₂CH₂OCH₃ | —H | —CH₃ |
| (33a) | 4-CONHCH₂CH₂OCH₃ | —H | —H |
| (34a) | 3-CONHCH₂COOH | —H | —H |
| (35a) | 3-CONHCH₂CN | —H | —H |
| (36a) | 3-OCH₂COOCH₃ | —H | —CH₃ |
| (37a) | 3-OCH₂COOH | —H | —CH₃ |
| (38a) | 3-OCH₂CON(CH₃)₂ | —H | —CH₃ |
| (39a) | 3-OCH₂CH₂CH₂N(CH₃)₂ | —H | —H |
| (40a) | 3-OCH₂CH₂OH | —H | —CH₃ |
| (41a) | 3-OCH₂CH₂OH | —CH₃ | —CH₃ |
| (42a) | 3-NHCOOCH₂CH₂OCH₃ | —H | —CH₃ |
| (43a) | 3-CH═CF₂ | —H | —H |
| (44a) | 3-CH₂CH₂CN | —H | —H |
| (45a) | 3-OCH₂CH₂SCH₃ | —H | —CH₃ |
| (46a) | 3-OCH₂CH₂SOCH₃ | —H | —CH₃ |
| (47a) | 3-OCH₂CH₂SO₂CH₃ | —H | —CH₃ |
| (48a) | 3-OCH₂CH₂OH | —H | —H |
| (49a) | 3-CH₂OCH₂CH₂OH | —H | —CH₃ |
| (50a) | 3-OCH₂CH₂CH₂OH | —H | —CH₃ |
| (51a) | 3-OCH₂CH₂OCH₃ | —H | —CH₃ |
| (52a) | 3-OCH₂CH₂NH₂ | —H | —CH₃ |
| (53a) | 3-OCH₂CH₂NHCOCH₃ | —H | —CH₃ |
| (54a) | 3-OCH₂CH₂NHCOOC(CH₃)₃ | —H | —CH₃ |
| (55a) | 3-OCH₂CH₂N(CH₃)₂ | —H | —H |
| (56a) | 3-OCH₂CH₂N(CH₃)₂ | —H | —CH₃ |
| (57a) | 3-OCH₂CH₂CH₂N(CH₃)₂ | —H | —CH₃ |
| (58a) | 3-OCH₂CH₂SO₃H | —H | —CH₃ |
| (59a) | 3-OCH₂CH₂CH₂SO₃Na | —Na | —H |
| (60a) | 3-OCH₂COOCH₃ | —CH₃ | —CH₃ |

TABLE 34

| Compound | | | |
|---|---|---|---|
| No. | Xₐ and Yₐ | rₐ | tₐ |
| (61a) | 3-OCH₂COO(CH₂)₉—OH | —H | —CH₃ |
| (62a) | 4-OCH₂COOCH₃ | —H | —H |
| (63a) | 3-OCH₂COOH•pyridine | —H | —H |
| (64a) | 3-OCH₂COOH | —CH₃ | —CH₃ |
| (65a) | 4-OCH₂COOH | —H | —H |
| (66a) | 3-OCH₂CONH₂ | —H | —H |
| (67a) | 3-OCH₂CONH₂ | —H | —CH₃ |
| (68a) | 3-OCH₂CONH₂ | —CH₃ | —CH₃ |
| (69a) | 3-OCH₂CON(CH₃)₂ | —H | —H |
| (70a) | 3-OCH₂CON(CH₃)₂ | —CH₃ | —CH₃ |
| (71a) | 3-BR,4-OCH₂COOCH₃ | —H | —H |
| (72a) | 3-CH₃,4-OCH₂COOCH₃ | —H | —H |
| (73a) | 3-CH₃,4-OCH₂COOCH₃ | —H | —CH₃ |
| (74a) | 3-NHCOCH₃,4-OCH₂CN | —H | —H |
| (75a) | 3-OCH₂COCH₃ | —H | —CH₃ |
| (76a) | 3-CH₂SCH₂COOCH₃ | —H | —CH₃ |
| (77a) | 3-CH₂SOCH₂COOCH₃ | —H | —CH₃ |
| (78a) | 3-CH₂SO₂CH₂COOCH₃ | —H | —CH₃ |
| (79a) | 3-NHCOCH₂OCH₃ | —CH₃ | —CH₃ |
| (80a) | 3-NHCOOCH₂CH₂OCH₃ | —CH₃ | —CH₃ |
| (81a) | 3-NHSO₂CH₂CH═CH₂ | —H | —CH₃ |
| (82a) | 3-NHCH₂CH₂N(CH₃)₂ | —H | —H |
| (83a) | 3-CONHCH₂COOCH₃ | —CH₃ | —CH₃ |
| (84a) | 4-CONHCH₂COOCH₃ | —H | —CH₃ |
| (85a) | 4-CONHCH₂COOCH₃ | —CH₃ | —CH₃ |
| (86a) | 3-CONHCH₂COOH | —H | —CH₃ |
| (87a) | 3-CONHCH₂COOH | —CH₃ | —CH₃ |
| (88a) | 4-CONHCH₂COOH | —H | —H |
| (89a) | 4-CONHCH₂COOH | —H | —CH₃ |
| (90a) | 4-CONHCH₂COOH | —CH₃ | —CH₃ |

TABLE 35

| Compound | | | |
|---|---|---|---|
| No. | Xₐ and Yₐ | ra | tₐ |
| (91a) | 3-CONHCH₂CONH₂ | —H | —H |
| (92a) | 3-CONHCH₂CONH₂ | —H | —CH₃ |
| (93a) | 3-CONHCH₂CONH₂ | —CH₃ | —CH₃ |
| (94a) | 3-CONHCH(CO₂CH₃)—CH₂CO₂CH₃ | —H | —H |
| (95a) | 3-CONHCH(CO₂H)—CH₂CO₂H | —H | —H |
| (96a) | 3-CONHCH₂CH₂OH | —H | —CH₃ |
| (97a) | 3-CONHCH₂CH₂OH | —CH₃ | —CH₃ |
| (98a) | 3-CONHCH₂CH₂OCH₃ | —CH₃ | —CH₃ |
| Compound | | | |
| No. | Compound (Va) | | |
| (99a) | 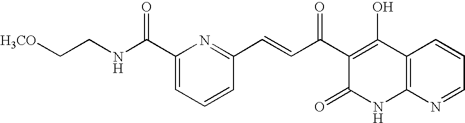 | | |
| Compound | | | |
| No. | Xₐ and Yₐ | rₐ | tₐ |
| (100a) | 3-CONHSO₂CH₃ | —H | —CH₃ |
| (101a) | 3-SO₂NHCH₂CH₂OCH₃ | —H | —H |
| (102a) | 3-SO₂NHCH₂CH₂OCH₃ | —H | —CH₃ |
| (103a) | 3-CONHOCH₃ | —H | —CH₃ |
| (104a) | 3-CONHOCH₂CH═CH₂ | —H | —CH₃ |
| Compound | | | |
| No. | compound (Va) | | |
| (105a) | 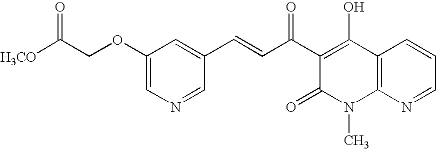 | | |
| Compound | | | |
| No. | Xₐ and Yₐ | rₐ | tₐ |
| (106a) | 3-CH₂CH₂CN | —H | —CH₃ |
| (107a) | 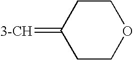 | —H | —CH₃ |

TABLE 36

| Compound | | | |
|---|---|---|---|
| No. | Xₐ and Yₐ | rₐ | tₐ |
| (108a) | 3-CH═CHCN | —H | —CH₃ |
| (109a) | 3-C≡CC(CH₃)₂OH | —H | —CH₃ |
| (110a) | 3-CH═CHCOOCH₃ | —H | —CH₃ |
| (111a) | 3-OCH₂CH═CH₂ | —H | —CH₃ |
| (112a) | 3-NHCOCOOCH₃ | —H | —CH₃ |
| (113a) | 3-CH═NOCH₃ | —H | —CH₃ |
| (114a) | 3-NHCSNHCH3 | —H | —CH₃ |
| (115a) | 3-N═C(—SCH₃)NHCH₃ | —H | —CH₃ |
| (116a) | 3-CH₂P(═O) (OCH₃)₂ | —H | —CH₃ |
| (117a) | 3-CH₂SOCH₂COOH | —H | —CH₃ |
| (118a) | 3-CH₂SO₂CH₂COOH | —H | —CH₃ |
| (119a) | 3-NHCOCOOH | —H | —CH₃ |
| (120a) | 3-OCH₂CH₂NH₂•HCl | —H | —CH₃ |
| (121a) | 3-CH₂P(═O) (OH)₂ | —H | —CH₃ |
| (122a) | 3-CH2COO(Ca)_{0.5} | —(Ca)_{0.5} | —CH₃ |
| (123a) | 3-CH2COONa | —Na | —CH₃ |

TABLE 37

| Compound | | | |
|---|---|---|---|
| No. | Xₐ and Yₐ | rₐ | tₐ |
| Compound | compound (Va) | | |
| No. | | | |
| (124a) | 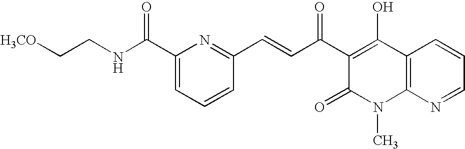 | | |
| (125a) | 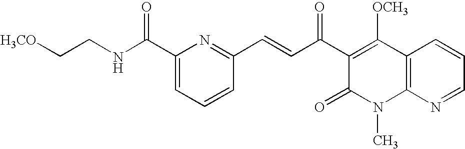 | | |
| (126a) | 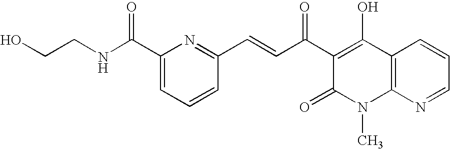 | | |
| (127a) | 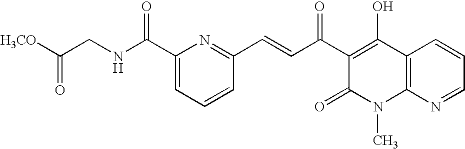 | | |
| (128a) | 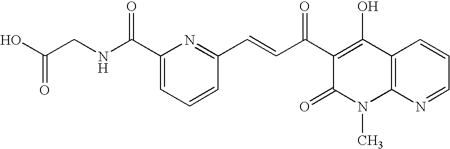 | | |
| (129a) | 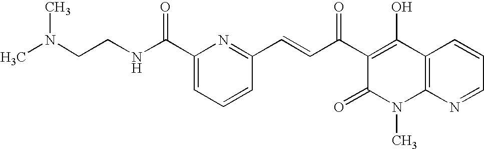 | | |

Among the compounds (V), examples of a compound represented by the formula (Vb):

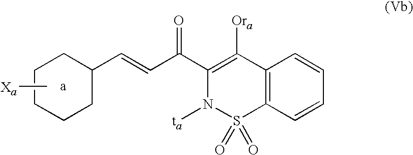

(wherein a, Xₐ, rₐ and tₐ are as defined above), which are represented by the compound numbers (1b) to (17b), are shown in Table 38 to Table 39.

### (Table 38 to Table 39)

In Table 38, in the compound numbers (1b) to (16b), a represents a benzene ring.

TABLE 38

| Compound | | | |
|---|---|---|---|
| No. | Xₐ | rₐ | tₐ |
| (1b) | —OCH₂COOCH₃ | —H | —CH₃ |
| (2b) | —OCH₂COOH | —H | —CH₃ |
| (3b) | —NHCOOCH₂CH₂OCH₃ | —H | —CH₃ |
| (4b) | —NHCONHCH₂CH₂OCH₃ | —H | —CH₃ |
| (5b) | —NHCOCH₂OCH₃ | —H | —CH₃ |
| (6b) | —NHCOCH₂OCH₃ | —CH₃ | —CH₃ |
| (7b) | —CONHCH₂CH₂OH | —H | —H |
| (8b) | —CONHCH₂CH₂OH | —H | —CH₃ |
| (9b) | —CONHCH₂CH₂OCH₃ | —H | —H |
| (10b) | —CONHCH₂CH₂OCH₃ | —H | —CH₃ |
| (11b) | —CONHCH₂CH₂OCH₃ | —CH₃ | —CH₃ |
| (12b) | —CONHCH₂CH₂N(CH₃)₂ | —H | —CH₃ |
| (13b) | —CONHCH₂COOCH₃ | —H | —CH₃ |
| (14b) | —CONHCH₂COOH | —H | —CH₃ |
| (15b) | —CONHCH₂CONH₂ | —H | —CH₃ |
| (16b) | —OCH₂CH₂OH | —H | —CH₃ |

TABLE 39

| | |
|---|---|
| Compound (Vb) Compound No. (17b) | 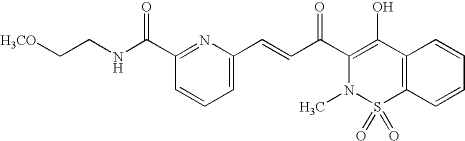 |

Among the compounds (V), examples of the compound (Vc) represented by the compound numbers (1c) to (26c) are shown in Tables 40 to 49.

### (Tables 40 to 49)

TABLE 40

| Compound | | |
|---|---|---|
| No. | Compound (Vc) | r |
| (1c) | 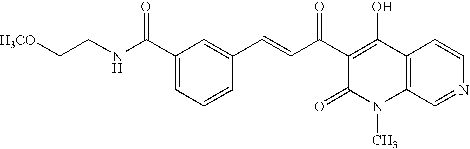 | |
| (2c) | 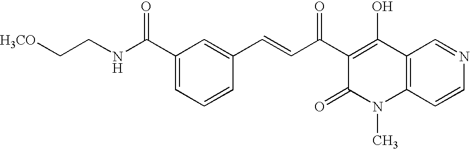 | |
| (3c) | 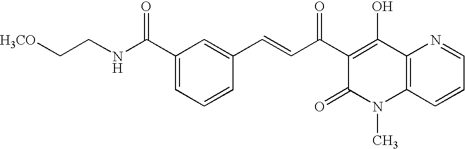 | |
| (4c) | 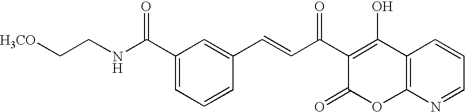 | |
| (5c) | 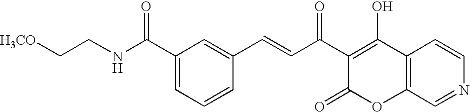 | |
| (6c) | 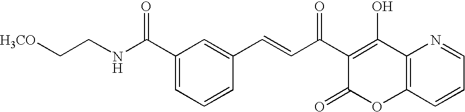 | |
| (7c) (8c) | 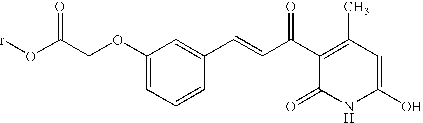 | —CH₃ —H |

TABLE 41

| | | |
|---|---|---|
| Compound | | |
| No. | Compound (Vc) | r |

| | | |
|---|---|---|
| (9c) (10c) | 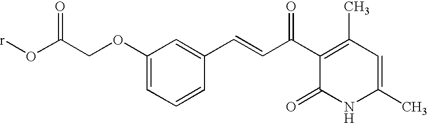 | —CH₃ —H |
| (11c) (12c) | 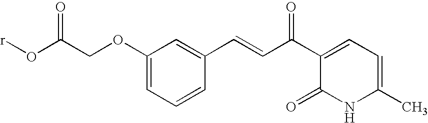 | —CH₃ —H |
| (13c) (14c) | 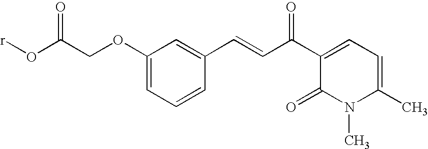 | —CH₃ —H |
| (15c) | 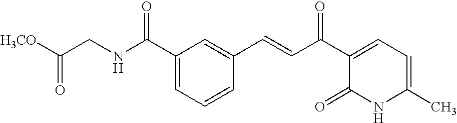 | |
| (16c) | 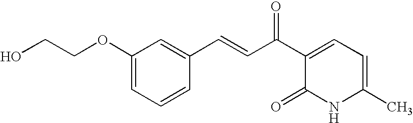 | |

TABLE 42

| Compound | | |
|---|---|---|
| No. | Compound (Vc) | r |
| (17c) (18c) | 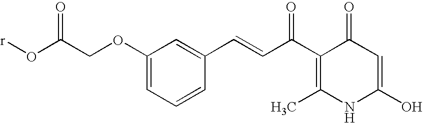 | —CH₃ —H |
| (19c) | 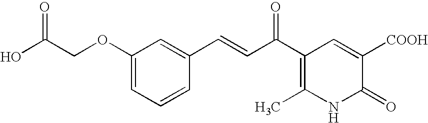 | |

TABLE 43

| | |
|---|---|
| compound (Vc) compound No. (20c) | 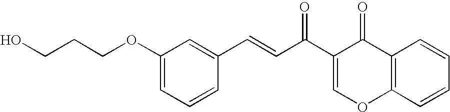 |

TABLE 44

| | |
|---|---|
| compound (Vc) compound No. (21c) | 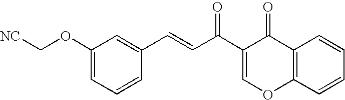 |

TABLE 45

| | |
|---|---|
| compound (Vc) compound No. (22c) | 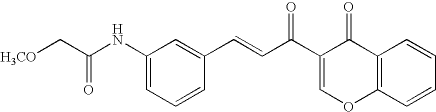 |

TABLE 46

| | |
|---|---|
| Compound (Vc) Compound No. (23c) | 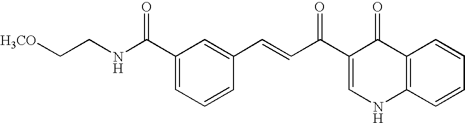 |

TABLE 47

| | |
|---|---|
| Compound (Vc) Compound No. (24c) | 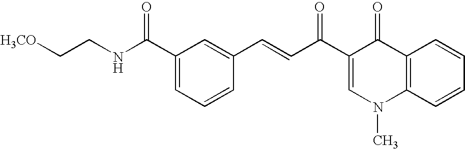 |

TABLE 48

| | |
|---|---|
| Compound (Vc) Compound No. (25c) | 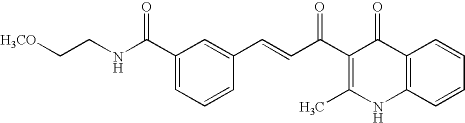 |

TABLE 49

| | |
|---|---|
| Compound (Vc) Compound No. (26c) | 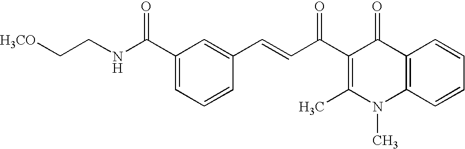 |

Among the compounds (V), Examples of the compound (Vd) represented by the compound numbers (1d) to (40d) are shown in Table 50 to Table 51.

### (Table 50 to Table 51)

### Compound (Vd)

TABLE 50

| | |
|---|---|
| | (Vd) |
| 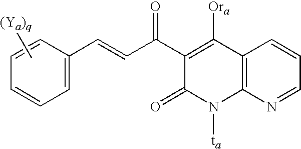 | |

| Compound | | | |
|---|---|---|---|
| No. | (Yₐ)_{q} | rₐ | tₐ |
| (1d) | H | H | H |
| (2d) | H | H | CH₃ |
| (3d) | H | CH₃ | CH₃ |
| (4d) | 3-Cl | H | H |
| (5d) | 3-CH₃ | H | H |
| (6d) | 4-CF₃ | H | H |
| (7d) | 3-CH₂OCH₃ | H | H |
| (8d) | 3-CH═CHCH₃ | H | H |
| (9d) | 3-C≡CH | H | H |
| (10d) | 3-OC₂H₅ | H | H |
| (11d) | 4-SCH₃ | H | H |
| (12d) | 4-S(O)CH₃ | H | H |
| (13d) | 4-S(O)₂CH₃ | H | H |
| (14d) | 4-NO₂ | H | H |
| (15d) | 3-CN | H | H |
| (16d) | 4-COOH | H | H |
| (17d) | 4-COOCH₃ | H | H |
| (18d) | 4-N(CH₃)₂ | H | H |
| (19d) | 3-NHCOCH₃ | H | H |
| (20d) | 3-NHCON(CH₃)₂ | H | H |
| (21d) | 3-CONH₂ | H | H |
| (22d) | 3-CON(CH₃)₂ | H | H |
| (23d) | 3-OCHF₂ | H | H |
| (24d) | 4-OCF₃ | H | H |
| (25d) | 4-OCF₂CHF₂ | H | H |
| (26d) | 2-SCF₃ | H | H |
| (27d) | 3,4-Cl₂ | H | H |
| (28d) | 2,4-(OCH₃)₂ | H | H |
| (29d) | 3-CH₃, 4-OCH₃ | H | H |
| (30d) | 3-OC₂H₅, 4-OH | H | H |

TABLE 51

| Compound | | | |
|---|---|---|---|
| No. | (Yₐ)_{q} | rₐ | tₐ |
| (31d) | 3-CF₃, 4-Cl | H | H |
| (32d) | 3-Cl, 4-OCF₃ | H | H |
| (33d) | 3-F, 4,5-(OCH₃)₂ | H | H |
| (34d) | 3-COOCH₃ | H | CH₃ |
| Compound No. | 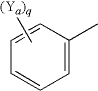 | rₐ | tₐ |
| (35d) | 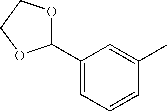 | H | CH₃ |
| (36d) | 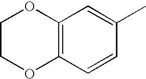 | H | CH₃ |
| (37d) | 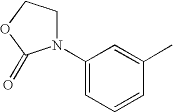 | H | H |
| (38d) | 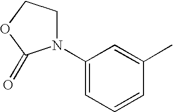 | H | CH₃ |
| (39d) | 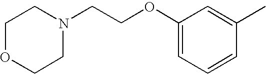 | H | H |
| (40d) | 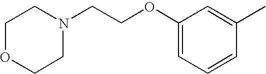 | H | CH₃ |

Among the compounds (V), examples of the compound (Ve) represented by the compound numbers (1e) to (3e) are shown in Table 52.

### (Table 52)

TABLE 52

| Compound | |
|---|---|
| No. | Compound (Ve) |
| (1e) | 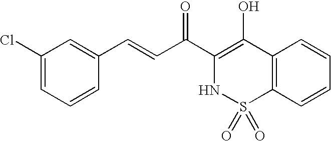 |
| (2e) | 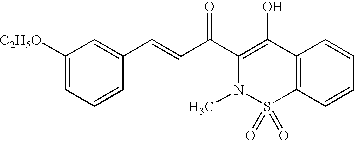 |
| (3e) | 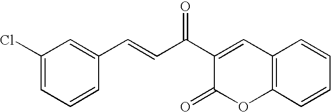 |

Among the compounds (V), examples of the compound (Vf) represented by the compound numbers (1f) to (15f) are shown in Table 53.

### (Table 53)

TABLE 53

Compound (Vf)

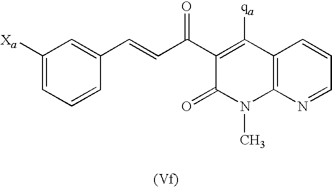

| Compound | | |
|---|---|---|
| No. | Xₐ | qₐ |
| (1f) | —OCH₂COOCH₃ | —OCH₂CH═CH₂ |
| (2f) | —OCH₂COOCH₃ | —OCH₂C≡CH |
| (3f) | —OCH₂COOCH₃ | —OCH₂COOCH₃ |
| (4f) | —OCH₂COOH | —OCH₂COOH |
| (5f) | —OCH₂CONH₂ | —OCH₂CONH₂ |
| (6f) | —OCH₂COOCH₃ | —OCH₂CN |
| (7f) | —OCH₂COOH | —OCH₂CH₂OH |
| (8f) | —OCH₂COOCH₃ | —OCH₂Ph |
| (9f) | —OCH₂COOH | —OCH₂Ph |
| (10f) | —OCH₂COOCH₃ | —OCH₂CH₂N(CH₃)₂ |
| (11f) | —OCH₂CH₂CH₂OH | 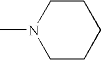 |
| (12f) | 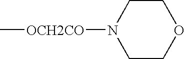 | 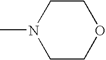 |
| (13f) | —OCH₂COOCH₃ | —NHCH₂C≡CH |
| (14f) | —OCH₂CONHCH₂CH₂OCH₃ | —NHCH₂CH₂OCH₃ |
| (15f) | —OCH₂COOH | —NHCH₂C≡CH |

Among the compounds (V), examples of the compound (Vg) represented by the compound numbers (1g) to (11g) are shown in Table 54.

### (Table 54)

TABLE 54

Compound (Vg)

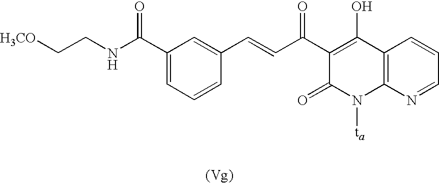

| Compound No. | tₐ |
|---|---|
| (1g) | —CH₂CH═CH₂ |
| (2g) | —CH₂C≡CH |
| (3g) | —CH₂COOCH₃ |
| (4g) | —CH₂COOH |
| (5g) | —CH₂CONH₂ |
| (6g) | —CH₂CN |
| (7g) | —CH₂COCH₃ |
| (8g) | —CH₂CH₂OCH₃ |
| (9g) | —CH₂Ph |
| (10g) | —Ph |
| (11g) | 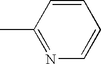 |

The compounds (I) to (V), (I′), (II′) and (V′) have the ability to suppress transcription of an extracellular matrix gene such as a Type I collagen gene or a fibronectin gene. The ability is important in improving tissue fibrosis because it decreases expression of an extracellular matrix gene such as a Type I collagen gene or a fibronectin gene to induce a reduction in accumulation of an extracellular matrix such as collagen or fibronectin. Therefore, the compounds (I) to (V), (I′), (II′) and (V′) can be utilized as an active ingredient of a composition (medicament, cosmetic, food additive etc.) which can improve tissue fibrosis by decreasing expression of an extracellular matrix gene such as a Type I collagen gene or a fibronectin gene to induce a reduction in accumulation of an extracellular matrix such as collagen or fibronectin.

A disease to which the transcription-suppressing composition of the present invention or the fibrosis-improving composition of the present invention can be applied includes, for example, a disease in which excessive accumulation of an extracellular matrix such as collagen or fibronectin causes fibrosis and then sclerosis of tissues, resulting in decreased function, cicatrization and the like in the tissues such as organs (i.e. fibrosing disease etc.). Specifically, examples of the disease include cirrhosis, chronic pancreatitis, scirrhous gastric cancer, interstitial pulmonary disease, asthma, chronic obstructive pulmonary diseases, glomerular nephritis, lupus nephritis, tubulointerstitial nephritis, IgA nephritis, renal sclerosis, diabetic nephritis, hereditary renal disease, myocardial fibrosis, heart failure, restenosis after PTCA, arterial sclerosis, marrow fibrosis, rheumatoid arthritis, hyperplasia scar after inflammation, postoperative scars or burn scars, atopic dermatitis, hypertrophic scar, hysteromyoma, prostate hypertrophy, scleroderma, Alzheimer disease, sclerotic peritonitis, diabetic retinopathy, I type diabetes, and the like. Incidentally, as an example of cirrhosis, it has been already known that C type or B type hepatitis virus induces chronic inflammation and then increased production of TGF-β, and thereby, hepatic fibrosis (particularly, accumulation of type I and type III collagen) is induced to cause cirrhosis (e.g. see Clin. Liver Dis., 7, 195-210 (2003)). As an example of interstitial pulmonary disease, it has been thought that pneumonia caused by mites, viruses, tubercle bacilli or the like induces increased production of TGF-β and then pulmonary fibrosis, and thereby interstitial pulmonary disease is caused. For chronic renal failure such as diabetic nephropathy and IgA nephropathy, it has been already suggested that diabetic nephropathy is caused by increased level of TGF-β in renal glomeruli due to hyperglycemia and thereby induction of renal fibrosis (particularly accumulation of Type I and Type IV collagen), and IgA nephropathy is caused by induction of nephritis due to accumulation of IgA in renal glomeruli followed by increased level of TGF-β, and thereby induction of renal fibrosis (particularly accumulation of Type I and Type IV collagen) (e.g. see Am. J. Physiol. Renal Physiol., 278, F830-F838 (2000), Kidney Int. 64, 149-159 (2003)). A db/db mouse, a diabetic nephropathy model animal, develops hyperglycemia by overeating because it has a mutation in a leptin receptor for suppressing ingestion, and then spontaneously develops diabetes. In the db/db mouse, the blood glucose concentration is about 4 times higher than a normal mouse, and fibrosis of renal glomeruli and increased level of TGF-β are found (e.g. see Am. J. Pathol., 158, 1653-1663 (2001)). An anti-Thy-1 rat, an IgA nephropathy model animal, is produced by administering an anti-Thy-1 antibody to a normal rat to artificially cause renal fibrosis. It has been shown that renal fibrosis is suppressed by administering an anti-TGF-β receptor antibody to the model animal (e.g. see Kidney Int., 60, 1745-1755 (2001)). Although the cause of scleroderma is unknown, it has been found that skin fibrosis is improved by administering a TGF-β inhibitor to a Tsk mouse, which is a model animal therefor (e.g. see J. Invest. Dermatol., 118.461-470 (2001)). Thus, a compound which suppresses the activity of TGF-β can be utilized as an active ingredient of a composition (medicament, cosmetic, food additive etc.) for inhibiting the collagen synthesis-promoting activity of TGF-β to suppress tissue fibrosis and thereby providing a fibrosing disease therapeutic effect. On the other hand, it is believed that a cause of heart failure such as left ventricular diastolic failure is cardiac fibrosis under a hypertensive condition. Thus, a compound which suppresses the activity of TGF-β can be utilized as an active ingredient of a composition (medicament etc.) for inhibiting the fibronectin synthesis-promoting activity of TGF-β to suppress tissue fibrosis and thereby providing a heart failure therapeutic effect.

Such transcription-suppressing composition or fibrosis-improving composition of the present invention comprises the compound (I) to (V), (I′), (II′) and (V′) and an inert carrier. Such composition usually comprises 0.01% by weight to 99.99% by weight of the compound (I) to (V), (I′), (II′) and (V′) and 99.99% by weight to 0.01% by weight of an inert carrier. The inert carrier is a pharmaceutically acceptable carrier or excipient. The transcription-suppressing composition and fibrosis-improving composition of the present invention may further comprise pharmaceutical additives, cosmetic additives, food additives and the like.

The compound (I) to (V), (I′), (II′) and (V′) also inhibits the ability of TGF-β to promote transcription of a Type I collagen gene or a fibronectin gene, as shown in Examples 3 to 5 below. That is, the compound (I) to (V), (I′), (II′) and (V′) is a TGF-β antagonist having the ability to suppress the activity of TGF-β. Therefore, the compound (I) to (V), (I′), (II′) and (V′) can be also utilized as an active ingredient of a composition for suppressing the activity of TGF-β.

Such TGF-β suppressing composition of the present invention comprises the compound (I) to (V), (I′), (II′) and (V′) and an inert carrier. Such composition usually comprises 0.01% by weight to 99.99% by weight of the compound (I) to (V), (I′), (II′) and (V′) and 99.99% by weight to 0.01% by weight of an inert carrier. The inert carrier is a pharmaceutically acceptable carrier or excipient. The TGF-β suppressing composition and hair-growing composition of the present invention may further comprise pharmaceutical additives, cosmetic additives, food additives and the like.

A pharmaceutically acceptable carrier, excipient, pharmaceutical additive, food additive, cosmetic additive, a medicament additive, and the like contained in the above-described composition can be appropriately selected depending on the specific use thereof. In addition, the composition may be in a form of various solids, liquids and the like depending on the specific use thereof.

For example, when the compound (I) to (V), (I′), (II′) and (V′) is used as an active ingredient of a medicament, specific examples of the medicament include oral preparations such as powders, fine granules, granules, tablets, syrups, capsules, suspensions, emulsions, extracts and pills; and parenteral preparations such as injections, transdermal absorbing agents such as external liquids and ointments, suppositories and local preparations.

Oral preparations can be prepared using carriers or excipients, and pharmaceutical additives such as binders, disintegrants, surfactants, lubricants, glidants, diluents, preservatives, coloring agents, flavors, stabilizers, humectants, antiseptics, antioxidants and the like, for example, gelatin, sodium alginate, starch, corn starch, white sugar, lactose, glucose, mannit, carboxymethylcellulose, dextrin, polyvinylpyrrolidone, crystalline cellulose, soybean lecithin, sucrose, fatty acid ester, talc, magnesium stearate, polyethylene glycol, magnesium silicate, anhydrous silicic acid and the like, according to a conventional method.

A dose of the oral preparation varies depending on the age, sex and weight of a mammal to be administered, the severity of disease, the kind and dosage form of the composition of the present invention, and the like. Usually, in the case of oral administration, about 1 mg to about 2 g per day, preferably about 5 mg to about 1 g per day of the active ingredient may be administered to an adult human. The daily dose may be also administered at one time or in several divided doses.

Among parenteral preparations, an injection can be prepared using such as a water-soluble solvent such as physiological saline or sterilized water Ringer solution, a water-insoluble solvent such as vegetable oil or fatty acid ester, an isotonic agent such as glucose or sodium chloride, pharmaceutical additives such as a solubilizer, a stabilizer, an antiseptic, a suspending agent and an emulsifying agent, and the like, according to a conventional method. A transdermal absorbing agent such as external liquid or a gel-like ointment, a suppository for rectal administration and the like can be also prepared according to a conventional method. For administering such parenteral preparations, they may be administered by injection (subcutaneously, intravenously etc.), transdermally, or rectally. The local agent can be produced, for example, by incorporating the compound (I) to (V), (I′), (II′) and (V′) into a pellet of a sustained-release polymer such as an ethylene vinyl acetate polymer. The pellet may be surgically transplanted into a tissue to be treated.

A dose of the parenteral preparation varies depending on the age, sex and weight of a mammal to be administered, the severity of disease, the kind and dosage form of the composition of the present invention, and the like. Usually, in the case of administration by injection, about 0.1 mg to about 500 mg of the active ingredient may be administered to an adult human. The daily dose may be also administered at one time or in several divided doses.

When the compound (I) to (V), (I′), (II′) and (V′) is used by adding to cosmetics, examples of specific forms of cosmetics with the compound added thereto include liquid, emulsion, cream, lotion, ointment, gel, aerosol, mousse and the like. Lotion can be prepared using cosmetic additives such as a suspending agent, an emulsifier, a preservative and the like, according to a conventional method.

A dose of the cosmetic varies depending on the age, sex and weight of a mammal to be administered, the severity of disease, the kind and dosage form of the composition of the present invention, and the like. Usually, about 0.01 mg to about 50 mg of the active ingredient may be administered to an adult human. The daily dose may be also administered at one time or in several divided doses.

When the compound (I) to (V), (I′), (II′) and (V′) is used as a food additive, examples of specific forms of a food with the addictive added thereto include powder, a tablet, a beverage, an edible gel or a mixed liquid of the gel and syrup, for example, general beverage and food and luxury food and beverage such as seasonings, Japanese confectioneries, western confectioneries, ice confectioneries, beverage, spreads, pastes, pickles, bottled or canned products, processed domestic animal meats, processed fish meats or marine product, processed dairy or egg products, processed vegetables, processed fruits, processed cereals and the like. Alternatively, the present compound can be also added to feeds or provenders for rearing animals such as livestocks, poultry, honey bee, silkworm, fish and the like.

A dose of the food varies depending on the age, sex and weight of a mammal to be administered, the severity of disease, the kind and dosage form of the composition of the present invention, and the like. Usually, about 0.1 mg to about 500 mg of the active ingredient may be administered to an adult human. The daily dose may be also administered at one time or in several divided doses.

### EXAMPLES

The following Examples further illustrate the present invention.

### Example 1

Synthesis of the compound (VII-3) represented by the compound numbers (1A-1) and (1A-5) shown in Table 30, and the compound (VII-2) represented by the compound number (1B-1) shown in Table 31 is described in Examples 1-1 to 1-3.

### Examples 1-1

### Synthesis of Compound (VII-3) [Compound No. (1A-1)]

To a solution of 1.49 g of methyl 2-(3-oxobutyrylamino)nicotinate in 35 mL of methanol was added 0.68 g of sodium methylate, and the mixture was heated under reflux for 30 minutes. Insolubles were filtered, washed with 40 mL of methanol and then dissolved in 150 mL of water, and 30 ml of an aqueous saturated ammonium chloride solution was added thereto. Insolubles were filtered, and the filtered crystal was washed with 60 ml of water, 40 ml of tetrahydrofuran and then 20 ml of hexane to obtain 1.00 g of 3-acetyl-4-hydroxy-1H-[1,8]naphthyridin-2-one [Compound No. (1A-1)] as a white solid.

¹H-NMR (270 MHz, DMSO-d₆) δ (ppm): 2.40 (s, 3H), 7.01 (dd, 1H, J=4.9, 7.6 Hz), 8.17 (dd, 1H, J=1.6, 7.6 Hz), 8.34 (dd, 1H, J=1.6, 4.9 Hz).

### Examples 1-2

### Synthesis of Compound (VII-3) [Compound No. (1A-5)]

To a mixture of 0.41 g of 3-acetyl-4-hydroxy-1H-[1,8]naphthyridin-2-one in 10 ml of dimethylformamide was added 0.12 g of sodium hydride (60% oily) at room temperature. After the mixture was stirred at room temperature for 1 hour, 0.4 ml of methyl iodide was added thereto, followed by stirring at room temperature overnight. Insolubes were filtered and washed with 10 ml of dimethylformamide, and 2 ml of an aqueous saturated ammonium chloride solution was added to the filtrate. The filtrate was concentrated under reduced pressure, and to the resulting residue was added 10 ml of tetrahydrofuran. Insolubles were filtered and washed with 15 ml of tetrahydrofuran, and the filtrate was concentrated under reduced pressure. The residue was subjected to silica gel column chromatography to obtain 0.06 g 3-acetyl-4-hydroxy-1-methyl-1H-[1,8]naphthyridin-2-one [Compound No. (1A-5)] as a white solid.

¹H-NMR (270 MHz, DMSO-d₆) δ (ppm): 2.74 (s, 3H), 3.64 (s, 3H), 7.39 (dd, 1H, J=4.6, 7.8 Hz), 8.48 (dd, 1H, J=1.9, 7.8 Hz), 8.80 (dd, 1H, J=1.9, 4.6 Hz), 16.86 (broad s, 1H).

### Examples 1-3

### Synthesis of Compound (VII-2) [Compound No. (1B-1)]

To a solution of 1.52 g of methyl 2-aminonicotinate in 3 ml of xylene was added 1.3 ml of 2,2,6-trimethyl-4H-1,3-dioxin-4-one, and the mixture was heated at 120° C. for 30 minutes. The reaction solution was subjected to silica gel column chromatography to obtain 1.15 g of methyl 2-(3-oxobutyrylamino)nicotinate [Compound No. (1B-1)] as a white solid.

¹H-NMR (270 MHz, DMSO-d₆) δ (ppm): 2.22 (s, 3H), 3.68 (s, 2H), 3.74 (s, 3H), 7.27˜7.32 (m, 1H), 8.10˜8.20 (m, 1H), 8.49˜8.51 (m, 1H)

### Example 2

Synthesis of the compound (Va) represented by the compound numbers (19a), (20a), (29a), (31a), (32a), (36a), (37a), (40a), (42a), (56a), (86a) and (124a) shown in Tables 32 to 37, the compound (Vb) represented by the compound numbers (1b), (2b), (5b), (9b), (10b), (13b), (14b), (16b) and (17b) shown in Table 38 to Table 39, the compound (Vc) represented by the compound numbers (9c) to (25c) shown in Tables 40 to 49, and the compound (Ve) represented by the compound number (3e) shown in Table 52 is described in Examples 2-1 to 2-39.

### Examples 2-1

### Synthesis of Compound (Va) [Compound No. (19a)]

To a solution of 97 mg of 3-(methoxyacetylamino)benzaldehyde in 3 ml of ethanol were added 109 mg of 3-acetyl-4-hydroxy-1-methyl-1H-[1,8]naphthyridin-2-one and 14 mg of piperidine, and the mixture was heated under reflux for 2 hours and 15 minutes. After cooling, the residue was filtered, and the filtered crystal was washed with 3 ml of ethyl acetate to obtain 68 mg of 4-hydroxy-1-methyl-3-[3-[3-(methoxyacetylamino)phenyl]acryloyl]-1H-[1,8]naphthyridin-2-one [Compound No. (19a)] as light brown powder.

¹H-NMR (300 MHz, DMSO-d₆) δ (ppm): 3.47 (s, 3H), 3.66 (s, 3H), 4.04 (s, 2H), 7.35˜7.44 (m, 1H), 7.44˜7.48 (2H), 7.80˜7.86 (m, 1H), 7.91 (d, 1H, J=15.9 Hz), 8.11 (s, 1H), 8.49 (dd, 1H, J=1.8, 7.8 Hz), 8.57 (d, 1H, J=15.9 Hz), 8.80 (dd, 1H, J=1.5, 4.5 Hz), 10.05 (broad, 1H)

### Examples 2-2

### Synthesis of Compound (Va) [Compound No. (20a)]

According to the same manner as that of Example 2-1 except that 0.09 g of 4-(methoxyacetylamino)benzaldehyde was used in place of 3-(methoxyacetylamino)benzaldehyde, 0.15 g of 4-hydroxy-1-methyl-3-[3-[4-(methoxyacetylamino)phenyl]acryloyl]-1H-[1,8]naphthyridin-2-one [Compound No. (20a)] was obtained as light brown powder.

¹H-NMR (270 MHz, DMSO-d₆) δ (ppm): 3.39 (s, 3H), 3.66 (s, 3H), 4.04 (s, 2H), 7.39 (dd, 1H, J=5.4, 8.1 Hz), 7.74 (d, 2H, J=8.1 Hz), 7.83 (d, 2H, J=8.1 Hz), 7.94 (d, 1H, J=16.2 Hz), 8.49 (d, 1H, J=8.1 Hz), 8.53 (d, 1H, J=13.5 Hz), 8.79 (d, 1H, J=5.4 Hz), 10.08 (s, 1H), 18.03 (broad, 1H)

### Examples 2-3

### Synthesis of Compound (Va) [Compound No. (29a)]

According to the same manner as that of Example 2-1 except that 0.20 g of 3-[[(methoxycarbonylmethyl)amino]carbonyl]benzaldehyde was used in place of 3-(methoxyacetylamino)benzaldehyde and 6 ml of methanol was used in place of ethanol, 0.19 g of 4-hydroxy-1-methyl-3-[3-[3-[[(methoxycarbonylmethyl)amino]carbonyl]phenyl}acryloyl]-1H-[1,8]naphthyridin-2-one [Compound No. (29a)] was obtained as a light brown crystal.

¹H-NMR (270 MHz, DMSO-d₆) δ (ppm): 3.67 (s, 3H), 3.68 (s, 3H), 4.06 (d, 2H, J=5.7 Hz), 7.40 (dd, 1H, J=4.9, 7.8 Hz), 7.62 (t, 1H, J=7.8 Hz), 7.94 (d, 1H, J=7.6 Hz), 7.97 (d, 1H, J=15.4 Hz), 7.99 (d, 1H, J=7.8 Hz), 8.26 (s, 1H), 8.50 (dd, 1H, J=1.6, 7.6 Hz), 8.60 (d, 1H, J=15.9 Hz), 8.81 (dd, 1H, J=1.6, 4.6 Hz), 9.16 (t, 1H, J=5.7 Hz)

### Examples 2-4

### Synthesis of Compound (Va) [Compound No. (31a)]

To a solution of 0.25 g of 3-[(2-methoxyethyl)aminocarbonyl]benzaldehyde in 5 ml of ethanol were added 0.20 g of 3-acetyl-4-hydroxy-1H-[1,8]naphthyridin-2-one and 0.03 ml of piperidine, and the mixture was heated under reflux for 30 minutes. Further, 5 ml of ethanol and 4 ml of dimethylformamide were added thereto, and the mixture was heated under reflux for 2 hours and 45 minutes. Then 15 ml of ethanol was added thereto and insolubles were filtered and recrystallized from 3 ml of dimethylformamide. The resulting crystal was washed with 10 ml of ethanol to obtain 0.07 g of 4-hydroxy-3-[3-[3-[(2-methoxyethyl)aminocarbonyl]phenyl}acryloyl]-1H-[1,8]naphthyridin-2-one [Compound No. (31a)] as a light yellow crystal.

¹H-NMR (270 MHz, DMSO-d₆) δ (ppm): 3.29 (s, 3H), 3.40˜3.60 (m, 4H), 7.30˜7.40 (m, 1H), 7.59 (t, 1H, J=7.3 Hz), 7.90 (d, 1H, J=7.8 Hz), 7.93 (d, 1H, J=8.4 Hz), 7.99 (d, 1H, J=16.5 Hz), 8.23 (s, 1H), 8.42 (d, 1H, J=7.8 Hz), 8.60˜8.75 (3H)

### Examples 2-5

### Synthesis of Compound (Va) [Compound No. (32a)]

To a solution of 0.10 g of 3-[(2-methoxyethyl)aminocarbonyl]benzaldehyde in 3 ml of ethanol were added 0.06 g of 3-acetyl-4-hydroxy-1-methyl-1H-[1,8] naphthyridin-2-one and 0.01 ml of piperidine, and the mixture was heated under reflux for 1 hour and 30 minutes. After concentration under reduced pressure, 3 ml of ethyl acetate was added to the residue. The mixture was filtered, and the filtered crystal was washed with 6 ml of ethyl acetate to obtain 0.07 g of 4-hydroxy-1-methyl-3-[3-[3-[(2-methoxyethyl)aminocarbonyl]phenyl}acryloyl]-1H-[1,8]naphthyridin-2-one [Compound No. (32a)] as a light yellow crystal.

¹H-NMR (270 MHz, DMSO-d₆) 5 (ppm): 3.29 (s, 3H), 3.40˜3.60 (m, 4H), 3.68 (s, 3H), 7.35˜7.45 (m, 1H), 7.59 (t, 1H, J=7.8 Hz), 7.85˜8.05 (3H), 8.24 (s, 1H), 8.51 (d, 1H, J=7.8 Hz), 8.58 (d, 1H, J=15.1 Hz), 8.71 (broad, 1H), 8.81 (d, 1H, J=4.1 Hz)

### Examples 2-6

### Synthesis of Compound (Va) [Compound NO. (36a)]

According to the same manner as that of Example 2-1 except that 0.18 g of 3-[(methoxycarbonyl)methoxy]benzaldehyde was used in place of 3-(methoxyacetylamino)benzaldehyde and 6 ml of methanol was used in place of ethanol, 0.23 g of 4-hydroxy-1-methyl-3-[3-[3-[(methoxycarbonyl)methoxy]phenyl]acryloyl]-1H-[1,8]naphthyridin-2-one [Compound No. (36a)] was obtained as a light yellow crystal.

¹H-NMR (270 MHz, DMSO-d₆) δ (ppm): 3.67 (s, 3H), 3.74 (s, 3H), 4.89 (s, 2H), 7.07˜7.11 (1H), 7.31 (s, 1H), 7.36˜7.46 (3H), 7.93 (d, 1H, J=16.5 Hz), 8.50 (dd, 1H, J=1.9, 7.6 Hz), 8.52˜8.58 (1H), 8.82 (d, 1H, J=3.8 Hz)

### Examples 2-7

### Synthesis of Compound (Va) [Compound No. (37a)]

To a solution of 0.15 g of 4-hydroxy-1-methyl-3-[3-[3-[(methoxycarbonyl)methoxy]phenyl]acryloyl]-1H-[1,8]naphthyridin-2-one in 4.6 ml of methanol was added 4.6 ml of a 1N aqueous sodium hydroxide solution. The mixture was stirred at room temperature for 2 hours and 15 minutes. The solvent was distilled off under reduced pressure and then acidified with 10% hydrochloric acid. Precipitated crystals were filtered, washed with ethyl acetate and hexane, and then dried to obtain 0.13 g of 4-hydroxy-1-methyl-3-[3-[3-(carboxymethoxy)phenyl]acryloyl]-1H-[1,8]naphthyridin-2-one [Compound No. (37a)] as a tan crystal.

¹H-NMR (270 MHz, DMSO-d₆) δ (ppm): 3.63 (s, 3H), 4.76 (s, 2H), 7.04 (d, 1H, J=7.6 Hz), 7.28 (s, 1H), 7.34˜7.44 (3H), 7.85 (d, 1H, J=15.9 Hz), 8.44˜8.50 (1H), 8.48 (dd, 1H, J=2.2, 7.8 Hz), 8.76˜8.78 (1H)

### Examples 2-8

### Synthesis of Compound (Va) [Compound No. (40a)]

According to the same manner as that of Example 2-1 except that 0.23 g of 3-(2-hydroxyethoxy)benzaldehyde was used in place of 3-(methoxyacetylamino)benzaldehyde, 0.19 g of 4-hydroxy-1-methyl-3-[3-[3-(2-hydroxyethoxy)phenyl]acryloyl]-1H-[1,8]naphthyridin-2-one [Compound No. (40a)] was obtained as a yellow crystal.

¹H-NMR (270 MHz, DMSO-d₆) δ (ppm): 3.32 (s, 3H), 3.72˜3.78 (2H), 4.05 (t, 2H, J=5.4 Hz), 7.08 (d, 1H, J=8.1 Hz), 7.29 (s, 1H), 7.32˜7.43 (3H), 7.91 (d, 1H, J=16.2 Hz), 8.48 (dd, 1H, J=2.7, 8.1 Hz), 8.54 (d, 1H, J=16.2 Hz), 8.79 (d, 1H)

### Examples 2-9

### Synthesis of Compound (Va) [Compound No. (42a)]

According to the same manner as that of Example 2-1 except that 0.10 g of 3-[(2-methoxyethoxy)carbonylamino]benzaldehyde was used in place of 3-(methoxyacetylamino)benzaldehyde, 0.11 g of 4-hydroxy-1-methyl-3-[3-[3-[(2-methoxyethoxy)carbonylamino]phenyl]acryloyl]-1H-[1,8]naphthyridin-2-one [Compound No. (42a)] was obtained as a tan crystal.

¹H-NMR (300 MHz, DMSO-d₆) δ (ppm): 3.30 (s, 3H), 3.59 (t, 2H, J=4.5 Hz), 3.65 (s, 3H), 4.24 (t, 2H, J=4.5 Hz), 7.33˜7.44 (3H), 7.57 (d, 1H, J=7.5 Hz), 7.89 (d, 1H, J=14.7 Hz), 7.94 (s, 1H), 8.45˜8.50 (1H), 8.56 (d, 1H, J=15.0 Hz), 8.77˜8.83 (1H), 9.97 (s, 1H)

### Examples 2-10

### Synthesis of Compound (Va) [Compound No. (56a)]

According to the same manner as that of Example 2-1 except that 0.26 g of 3-(2-dimethylaminoethoxy)benzaldehyde was used in place of 3-(methoxyacetylamino)benzaldehyde, 0.22 g of 4-hydroxy-1-methyl-3-[3-[3-(2-dimethylaminoethoxy)phenyl]acryloyl]-1H-[1,8]naphthyridin-2-one [Compound No. (56a)] was obtained as a tan crystal.

¹H-NMR (270 MHz, DMSO-d₆) δ (ppm): 2.53 (s, 6H), 3.05 (t, 2H, J=5.4 Hz), 3.58 (s, 3H), 4.23 (t, 2H, J=5.4 Hz), 7.02 (d, 1H, J=10.8 Hz), 7.23 (d, 1H, J=8.1 Hz), 7.24 (s, 1H), 7.27 (d, 1H, J=8.1 Hz), 7.36 (t, 1H, J=8.1 Hz), 7.60 (d, 1H, J=16.2 Hz), 8.10 (d, 1H, J=16.2 Hz), 8.39 (dd, 1H, J=2.7, 5.4 Hz), 8.63 (d, 1H)

### Examples 2-11

### Synthesis of Compound (Va) [Compound No. (86a)]

According to the same manner as that of Example 2-7 except that 0.11 g of 4-hydroxy-1-methyl-3-[3-[3-[[(methoxycarbonylmethyl)amino]carbonyl]phenyl}acryloyl]-1H-[1,8]naphthyridin-2-one was used in place of 4-hydroxy-1-methyl-3-[3-[(methoxycarbonyl)methoxy]phenyl]acryloyl-1H-[1,8]naphthyridin-2-one, 0.12 g of 4-hydroxy-1-methyl-3-[3-[3-[[(carboxymethyl)amino]carbonyl]phenyl}acryloyl]-1H-[1,8]naphthyridin-2-one [Compound No. (86a)] was obtained as a yellow crystal.

¹H-NMR (270 MHz, DMSO-d₆) δ (ppm): 3.67 (s, 3H), 3.97 (d, 1H, J=5.9 Hz), 7.35˜7.45 (1H), 7.62 (t, 1H, J=7.8 Hz), 7.80˜8.10 (3H), 8.27 (s, 1H), 8.40˜8.55 (1H), 8.60 (d, 1H, J=14.6 Hz), 8.80˜8.90 (1H), 9.07 (1H)

### Examples 2-12

### Synthesis of Compound (Va) [Compound No. (124a)]

According to the same manner as that of Example 2-1 except that 0.10 g of 6-formyl-2-[(2-methoxyethyl)aminocarbonyl]pyridine was used in place of 3-(methoxyacetylamino)benzaldehyde, 0.05 g of 4-hydroxy-1-methyl-3-[3-[6-(2-methoxyethyl)aminocarbonyl-2-pyridinyl]acryloyl]-1H-[1,8]naphthyridin-2-one [Compound No. (124a)] was obtained as a light yellow crystal.

¹H-NMR (300 MHz, DMSO-d₆) δ (ppm): 3.34 (s, 3H), 3.53˜3.54 (m, 1H), 3.66 (s, 3H), 7.34˜7.42 (1H), 7.90 (d, 1H, J=15.9 Hz), 8.02 (dt, 1H, J=1.5, 7.2 Hz), 8.06˜8.13 (2H), 8.42 (dd, 1H, J=2.1, 8.1 Hz), 8.67 (s, 1H), 8.76˜8.81 (1H), 8.80 (dd, 1H, J=1.8, 4.8 Hz)

### Examples 2-13

### Synthesis of Compound (Vb) [Compound No. (1b)]

To a solution of 1.53 g of 3-[(methoxycarbonyl)methoxy]benzaldehyde in 20 ml of methanol were added 2.00 g of 3-acetyl-4-hydroxy-2-methyl-2H-1,2-benzothiazine-1,1-dioxide and 202 mg of piperidine, and the mixture was heated under reflux for 9 hours and 10 minutes. After cooling, insolubles were filtered. The resulting crystal was washed with 10 ml of methanol and then dried to obtain 2.35 g of 4-hydroxy-3-[3-[3-[(methoxycarbonyl)methoxy]phenyl]acryloyl]-2-methyl-2H-1,2-benzothiazine-1,1-dioxide [Compound No. (1b)] as a light yellow crystal.

¹H-NMR (270 MHz, DMSO-d₆) δ (ppm): 2.97 (s, 3H), 3.73 (s, 3H), 4.91 (s, 2H), 7.08˜7.10 (m, 1H), 7.38˜7.54 (m, 4H), 7.89 (d, 1H, J=13.5 Hz), 7.95˜8.00 (m, 3H), 8.16˜8.20 (m, 1H)

### Examples 2-14

### Synthesis of Compound (Vb) [Compound No. (2b)]

To a solution of 300 mg of 4-hydroxy-3-[3-[3-(methoxycarbonylmethoxy)phenyl]acryloyl]-2-methyl-2H-1,2-benzothiazine-1,1-dioxide in 8 ml of methanol was added 8 ml of a 1N aqueous sodium hydroxide. The mixture was stirred at room temperature for 50 minutes. The solvent was distilled off under reduced pressure and then acidified with 2N hydrochloric acid. Precipitated crystals were filtered, washed with water and hexane and then dried to obtain 292 mg of 4-hydroxy-3-[3-[3-(carboxymethyl)phenyl]acryloyl]-2-methyl-2H-1,2-benzothiazine-1,1-dioxide [Compound No. (2b)] as a yellow crystal.

¹H-NMR (270 MHz, DMSO-d₆) δ (ppm): 2.97 (s, 3H), 4.79 (s, 2H), 7.06˜7.09 (m, 1H), 7.38˜7.53 (m, 4H), 7.69 (d, 1H, J=15.9 Hz), 7.97˜7.98 (m, 3H), 8.15˜8.18 (m, 1H)

### Examples 2-15

### Synthesis of Compound (Vb) [Compound No. (5b)]

According to the same manner as that of Example 2-13 except that 763 mg of 3-(methoxyacetylamino)benzaldehyde was used in place of 3-[(methoxycarbonyl)methoxy]benzaldehyde and 10 ml of ethanol was used in place of methanol, 1.28 g of 4-hydroxy-3-[3-[3-(methoxyacetylamino)phenyl]acryloyl]-2-methyl-2H-1,2-benzothiazine-1,1-dioxide [Compound No. (5b)] was obtained as a yellow crystal.

¹H-NMR (270 MHz, DMSO-d₆) δ (ppm): 2.97 (s, 3H), 3.41 (s, 3H), 4.04 (s, 2H), 7.34 (d, 1H, J=16.2 Hz), 7.44 (t, 1H, J=8.1 Hz), 7.58 (d, 1H, J=8.1 Hz), 7.86 (d, 1H, J=16.2 Hz), 7.89˜7.98 (m, 4H), 8.08 (s, 1H), 8.16 (m, 1H), 9.93 (s, 1H)

### Examples 2-16

### Synthesis of Compound (Vb) [Compound No. (9b)]

To a solution of 3-[(2-methoxyethyl)aminocarbonyl]benzaldehyde in 30 ml of ethanol were added 1.50 g of 3-acetyl-4-hydroxy-2H-1,2-benzothiazine-1,1-dioxide and 163 μl of piperidine, and the mixture was heated under reflux for 6 hours and 45 minutes. After cooling, 50 ml of 10% hydrochloric acid was added thereto, and the mixture was extracted with chloroform. An organic layer was washed with an aqueous saturated sodium chloride solution, dried over anhydrous sodium sulfate, and then concentrated under reduced pressure. Ethanol was added to the residue to induce crystallization and thereby 23 mg of 3-[3-[3-[(2-methoxyethyl)aminocarbonyl]phenyl}acryloyl]-4-hydroxy-2H-1,2-benzothiazine-1,1-dioxide (Compound No. (9b)] was obtained as a light yellow crystal.

¹H-NMR (270 MHz, DMSO-d₆) δ (ppm): 3.29 (s, 3H), 3.45˜3.55 (m, 4H), 7.55 (d, 1H, J=15.9 Hz), 7.58 (t, 1H, J=7.8 Hz), 7.85˜7.95 (6H), 8.15˜8.20 (1H), 8.29 (s, 1H), 8.74 (1H), 10.30 (broad, 1H), 15.69 (broad, 1H)

### Examples 2-17

### Synthesis of Compound (Vb) [Compound No. (10b)]

According to the same manner as that of Example 2-16 except that 1.11 g of 3-acetyl-4-hydroxy-2-methyl-2H-1,2-benzothiazine-1,1-dioxide was used in place of 3-acetyl-4-hydroxy-2H-1,2-benzothiazine-1,1-dioxide, 1.06 g of 4-hydroxy-3-[3-[3-[(2-methoxyethyl)aminocarbonyl]phenyl}acryloyl]-2-methyl-2H-1,2-benzothiazine-1,1-dioxide [Compound No. (10b)] was obtained as a yellow crystal.

¹H-NMR (270 MHz, DMSO-d₆) δ (ppm): 2.98 (s, 3H), 3.29 (s, 3H), 3.45˜3.55 (m, 4H), 7.48 (d, 1H, J=16.2 Hz), 7.59 (t, 1H, J=7.8 Hz), 7.90˜8.20 (5H), 8.28 (s, 1H), 8.70 (broad s, 1H), 15.73 (broad, 1H)

### Examples 2-18

### Synthesis of Compound (Vb) [Compound No. (13b)]

According to the same manner as that of Example 2-13 except that 1.75 g of 3-[[(methoxycarbonylmethyl)amino]carbonyl]benzaldehyde was used in place of 3-[(methoxycarbonyl)methoxy]benzaldehyde, 2.38 g of 4-hydroxy-3-[3-[3-[[(methoxycarbonylmethyl)amino]carbonyl]phenyl}acryloyl]-2-methyl-2H-1,2-benzothiazine-1,1-dioxide [Compound No. (13b)] was obtained as a yellow crystal.

¹H-NMR (270 MHz, DMSO-d₆) δ (ppm): 2.98 (s, 3H), 3.68 (s, 3H), 4.08 (d, 2H, J=5.7 Hz), 7.59 (d, 1H, J=16.2 Hz), 7.62 (t, 1H, J=7.6 Hz), 7.93-7.99 (m, 5H), 8.09 (d, 1H, J=7.6 Hz), 8.16˜8.19 (m, 1H), 8.33 (s, 1H), 9.16 (t, 1H, J=5.7 Hz)

### Examples 2-19

### Synthesis of Compound (Vb) [Compound No. (14b)]

According to the same manner as that of Example 2-14 except that 300 mg of 4-hydroxy-3-[3-[3-[[(methoxycarbonylmethyl)amino]carbonyl]phenyl}acryloyl]-2-methyl-2H-1,2-benzothiazine-1,1-dioxide was used in place of 4-hydroxy-3-[3-[3-[(methoxycarbonyl)methoxy]phenyl]acryloyl]-2-methyl-2H-1,2-benzothiazine-1,1-dioxide, 263 mg of 4-hydroxy-3-[3-[3-[[(carboxymethyl)amino]carbonyl]phenyl}acryloyl]-2-methyl-2H-1,2-benzothiazine-1,1-dioxide [Compound No. (14b)] was obtained as a yellow crystal.

¹H-NMR (300 MHz, DMSO-d₆) δ (ppm): 2.98 (s, 3H), 3.99 (d, 2H, J=5.7 Hz), 7.50 (d, 1H, J=16.2 Hz), 7.62 (t, 1H, J=7.8 Hz), 7.94˜7.98 (m, 5H), 8.09 (d, 1H, J=7.2 Hz), 8.16˜8.18 (m, 1H), 8.33 (s, 1H), 9.06 (t, 1H, J=5.7 Hz)

### Examples 2-20

### Synthesis of Compound (Vb) [Compound No. (16b)]

According to the same manner as that of Example 2-13 except that 656 mg of 3-(2-hydroxyethoxy)benzaldehyde was used in place of 3-[(methoxycarbonyl)methoxy]benzaldehyde and 10 ml of ethanol was used in place of methanol, 547 mg of 4-hydroxy-3-[3-[3-(2-hydroxyethoxy)phenyl]acryloyl]-2-methyl-2H-1,2-benzothiazine-1,1-dioxide [Compound No. (16b)] was obtained as a yellow crystal.

¹H-NMR (270 MHz, CDCl₃) δ (ppm): 2.05 (broad s, 1H), 3.00 (s, 3H), 4.03 (s, 2H), 4.17 (t, 1H, J=5.4 Hz), 7.03 (d, 1H, J=8.1 Hz), 7.29 (s, 1H), 7.33 (d, 1H, J=16.2 Hz), 7.37 (t, 1H, J=8.1 Hz), 7.75˜7.83 (m, 2H), 7.84 (d, 1H, J=16.2 Hz), 7.87˜7.94 (m, 1H), 8.15˜8.20 (m, 1H)

### Examples 2-21

### Synthesis of Compound (Vb) [Compound No. (17b)]

According to the same manner as that of Example 2-13 except that 1.64 g of 6-formyl-2-[(2-methoxyethyl)aminocarbonyl]pyridine was used in place of 3-[(methoxycarbonyl)methoxy]benzaldehyde, 2.70 g of 4-hydroxy-3-[3-[6-(2-methoxyethyl)aminocarbonyl-2-pyridinyl]acryloyl]-2-methyl-2H-1,2-benzothiazine-1,1-dioxide [Compound No. (17b)] was obtained as a yellow crystal.

¹H-NMR (270 MHz, CDCl₃) δ (ppm): 3.05 (s, 3H), 3.62 (s, 3H), 3.66 (t, 2H, J=5.4 Hz), 3.73 (t, 2H, J=5.4 Hz), 7.69 (dd, 1H, J=2.7, 8.1 Hz), 7.75˜7.96 (m, 6H), 8.17˜8.22 (m, 1H), 8.24 (d, 1H, J=8.1 Hz), 8.37 (broad s, 1H)

### Examples 2-22

### Synthesis of Compound (Vc) [Compound No. (9c)]

In 105 ml of pyridine were dissolved 10.50 g of 3-[(methoxycarbonyl)methoxy]benzaldehyde, 2.97 g of 3-acetyl-4,6-dimethyl-2(1H)-pyridinone and 2.38 g of piperidine, and the solution was heated under reflux for 14 hours. The solvent was distilled off under reduced pressure and the residue was subjected to silica gel column chromatography and then recrystallized from tetrahydrofuran to obtain 364 mg of 3-[3-[3-[(methoxycarbonyl)methoxy]phenyl]-1-oxo-2-propenyl]-4,6-dimethyl-2(1H)-pyridinone [Compound No. (9c)] as a light yellow crystal.

¹H-NMR (270 MHz, DMSO-d₆) δ (ppm): 2.08 (s, 3H), 2.18 (s, 3H), 3.70 (s, 3H), 4.86 (s, 2H), 6.01 (s, 1H), 6.99 (d, 1H, J=7.6 Hz), 7.15 (d, 1H, J=15.9 Hz), 7.28˜7.35 (3H), 7.38 (d, 1H, J=15.9 Hz), 11.83 (broad, 1H)

### Examples 2-23

### Synthesis of Compound (Vc) [Compound No. (10c)]

To a solution of 0.40 g of 3-[3-[3-[(methoxycarbonyl)methoxy]phenyl]-1-oxo-2-propenyl]-4,6-dimethyl-2(1H)-pyridinone in 10 ml of methanol was added 5 ml of a 1N aqueous sodium hydroxide solution. The mixture was stirred at room temperature for 30 minutes, and 2 ml of 10% hydrochloric acid was added thereto. Precipitated crystals were filtered and washed with a mixed solvent of methanol and water to obtain 0.27 g of 3-[3-[3-(carboxymethoxy)phenyl]-1-oxo-2-propenyl]-4,6-dimethyl-2(1H)-pyridinone [Compound No. (10c)] as light yellow powder.

¹H-NMR (270 MHz, DMSO-d₆) δ (ppm): 2.08 (s, 3H), 2.20 (s, 3H), 3.55 (broad, 1H), 4.74 (s, 2H), 6.01 (s, 1H), 6.97 (d, 1H, J=8.4 Hz), 7.15 (d, 1H, J=15.9 Hz), 7.25˜7.35 (m, 3H), 7.38 (d, 1H, J=16.2 Hz), 11.86 (broad, 1H)

### Examples 2-24

### Synthesis of Compound (Vc) [Compound No. (11c)]

According to the same manner as that of Example 2-22 except that 740 mg of 3-acetyl-6-methyl-2(1H)pyridinone was used in place of 3-acetyl-4,6-dimethyl-2-(1H)-pyridinone, 3-[3-[3-[(methoxycarbonyl)methoxy]phenyl]-1-oxo-2-propenyl]-6-methyl-2(1H)-pyridinone [Compound No. (11c)] was obtained as a light yellow crystal.

¹H-NMR (270 MHz, DMSO-d₆) δ (ppm): 2.27 (s, 3H), 3.72 (s, 3H), 4.86 (s, 2H), 6.24 (d, 1H, J=7.6 Hz), 6.98˜7.05 (1H), 7.23 (s, 1H), 7.28˜7.40 (2H), 7.58 (d, 1H, J=15.9 Hz), 8.07 (1H), 8.08 (d, 1H, J=16.5 Hz), 12.28 (broad, 1H)

### Examples 2-25

### Synthesis of Compound (Vc) [Compound No. (12c)]

In 30 ml of ethanol were dissolved 1.04 g of 3-[(methoxycarbonyl)methoxy]benzaldehyde and 0.74 g of 3-acetyl-6-methyl-2(1H)-pyridinone, and 10 ml of a 1N aqueous sodium hydroxide solution was added thereto. The mixture was stirred at room temperature for 5 hours and 30 minutes, and 10% hydrochloric acid was added thereto. Precipitated crystals were filtered and washed with water and tetrahydrofuran to obtain 3-[3-[3-(carboxymethoxy)phenyl]-1-oxo-2-propenyl]-6-methyl-2(1H)pyridinone (Compound No. (12c)].

¹H-NMR (270 MHz, DMSO-d₆) δ (ppm): 2.29 (s, 3H), 4.74 (s, 2H), 6.24 (d, 1H, J=7.6 Hz), 6.99 (d, 1H, J=7.0 Hz), 7.22 (s, 1H), 7.25˜7.40 (2H), 7.58 (d, 1H, J=15.9 Hz), 8.07 (1H), 8.08 (d, 1H, J=16.5 Hz), 12.29 (broad, 1H), 13.03 (broad, 1H)

### Examples 2-26

### Synthesis of Compound (Vc) [Compound No. (13c)]

To a mixture of 0.53 g of 3-[3-[3-[(methoxycarbonyl)methoxy]phenyl]-1-oxo-2-propenyl]-6-methyl-2(1H)-pyridinone and 15 ml of hexamethylphosphoramide was added 71 mg of sodium hydride (60% oily). After the mixture was stirred at room temperature for 1 hour, 0.15 ml of methyl iodide was added thereto. The mixture was stirred at room temperature for 4 hours. The reaction solution was poured into water, followed by extraction with ethyl acetate. The extract was dried over anhydrous sodium sulfate, and then concentrated under reduced pressure. The resulting residue was washed with a mixed solvent of hexane and t-butyl methyl ether to obtain 0.51 g of 3-[3-[3-[(methoxycarbonyl)methoxy]phenyl]-1-oxo-2-propenyl]-1,6-dimethyl-2(1H)-pyridinone [Compound No. (13c)] as a yellow crystal.

¹H-NMR (270 MHz, DMSO-d₆) δ (ppm): 2.49 (s, 3H), 3.53 (s, 3H), 3.72 (s, 3H), 4.86 (s, 2H), 6.38 (d, 1H), 6.99 (d, 1H), 7.24 (s, 1H), 7.29˜7.37 (2H), 7.57 (d, 1H), 7.99 (1H), 8.00 (d, 1H)

### Examples 2-27

### Synthesis of Compound (Vc) [Compound No. (14c)]

According to the same manner as that of Example 2-23 except that 0.46 g of 3-[3-[3-[(methoxycarbonyl)methoxy]phenyl]-1-oxo-2-propenyl]-1,6-dimethyl-2(1H)-pyridinone was used in place of 3-[3-[3-[(methoxycarbonyl)methoxy]phenyl]-1-oxo-2-propenyl]-4,6-dimethyl-2(1H)-pyridinone, 3-[3-[3-(carboxymethoxy)phenyl]-1-oxo-2-propenyl]-1,6-methyl-2(1H)-pyridinone [Compound No. (14c)] was obtained.

¹H-NMR (270 MHz, DMSO-d₆) δ (ppm): 2.49 (s, 3H), 3.53 (s, 3H), 4.73 (s, 2H), 6.38 (d, 1H, J=7.6 Hz), 6.99 (d, 1H, J=7.6 Hz), 7.22 (s, 1H), 7.25˜7.40 (2H), 7.57 (d, 1H, J=15.9 Hz), 7.97 (d, 1H, J=7.3 Hz), 7.99 (d, 1H, J=15.9 Hz)

### Examples 2-28

### Synthesis of Compound (Vc) [Compound No. (15c)]

According to the same manner as that of Example 2-24 except that 0.47 g of 3-[[(methoxycarbonylmethyl)amino]carbonyl]benzaldehyde was used in place of 3-[(methoxycarbonyl)methoxy]benzaldehyde, 0.19 g of 3-[3-[3-[[(methoxycarbonylmethyl)amino]carbonyl]phenyl}-1-oxo-2-propenyl]-6-methyl-2(1H)-pyridinone [Compound No. (15c)] was obtained as a light yellow crystal.

¹H-NMR (300 MHz, DMSO-d₆) δ (ppm): 2.30 (s, 3H), 3.70 (s, 3H), 4.35 (2H), 6.25 (d, 1H), 7.48˜7.70 (3H), 7.70˜8.00 (2H), 8.00˜8.22 (3H), 9.11 (1H), 11.93 (1H)

### Examples 2-29

### Synthesis of Compound (Vc) [Compound No. (16c)]

To 50 ml of ethanol were added 5.76 g of 3-(2-hydroxyethoxy)benzaldehyde and 1.94 g of 3-acetyl-6-methyl-2(1H)-pyridinone, and 25 ml of a 1N aqueous sodium hydroxide solution and 15 ml of water were added thereto. The mixture was heated under reflux for 2 hours, extracted with chloroform, and then dried over anhydrous sodium sulfate. The solvent was distilled off under reduced pressure, and the residue was subjected to silica gel column chromatography to obtain 0.66 g of 3-[3-[3-(2-hydroxyethoxy)phenyl]-1-oxo-2-propenyl]-6-methyl-2(1H)-pyridinone [Compound No. (16c)] as a yellow crystal.

¹H-NMR (270 MHz, DMSO-d₆) δ (ppm): 2.29 (s, 3H), 3.70˜3.76 (m, 1H), 4.03 (t, 1H, J=4.6 Hz), 4.90 (t, 1H, J=5.7 Hz), 6.24 (d, 1H, J=7.6 Hz), 6.90˜7.40 (4H), 7.68 (d, 1H, J=15.9 Hz), 8.07 (d, 1H, J=7.3 Hz), 8.08 (d, 1H, J=15.6 Hz), 12.27 (broad, 1H)

### Examples 2-30

### Synthesis of Compound (Vc) [Compound No. (17c)]

In 7 ml of chloroform were dissolved 0.85 g of 3-acetyl-6-hydroxy-2-methyl-1H-pyridin-4-one, 0.99 g of 3-[(methoxycarbonyl)methoxy]benzaldehyde and 0.35 g of piperidine. The solution was heated under reflux for 1 hour and 30 minutes while water was removed with a Soxhlet's extractor filled with molecular sieves, and then concentrated under reduced pressure. The residue was subjected to silica gel column chromatography, and then recrystallized from toluene to obtain 317 mg of 6-hydroxy-2-methyl-3-[3-[3-[(methoxycarbonyl)methoxy]phenyl]acryloyl]-1H-pyridin-4-one [Compound No. (17c)] as a light yellow crystal.

¹H-NMR (270 MHz, CDCl₃) δ (ppm): 2.02 (s, 3H), 3.82 (s, 3H), 4.66 (s, 2H), 5.19 (broad, 1H), 5.24 (s, 1H), 6.65 (d, 1H, J=15.9 Hz), 6.85˜6.88 (1H), 7.05˜7.35 (3H), 7.45 (d, 1H, J=15.9 Hz), 10.22 (broad, 1H)

### Examples 2-31

### Synthesis of Compound (Vc) [Compound No. (18c)]

According to the same manner as that of Example 2-23 except that 6-hydroxy-2-methyl-3-[3-[3-[(methoxycarbonyl)methoxy]phenyl]acryloyl]-1H-pyridin-4-one was used in place of 3-[3-[3-[(methoxycarbonyl)methoxy]phenyl]-1-oxo-2-propenyl]-4,6-dimethyl-2(1H)-pyridinone, 110 mg of 6-hydroxy-2-methyl-3-[3-[3-(carboxymethoxy)phenyl]acryloyl]-1H-pyridin-4-one [Compound No. (18c)] was obtained as a light yellow crystal.

¹H-NMR (270 MHz, CDCl₃) δ (ppm): 2.02 (s, 3H), 4.62 (s, 2H), 5.21 (s, 1H), 5.99 (broad, 1H), 6.64 (d, 1H, J=15.9 Hz), 6.87˜6.91 (1H), 7.09 (1H), 7.13˜7.36 (2H), 7.42 (d, 1H, J=15.7 Hz), 10.20 (broad, 1H)

### Examples 2-32

### Synthesis of Compound (Vc) [Compound No. (19c)]

Four grams of potassium hydroxide, 100 g of ethanol and 60 g of water were mixed to prepare a solution. To 22 g of the solution were added 0.36 g of 3-(cyanomethoxy)benzaldehyde and 0.35 g of 5-acetyl-6-methyl-2-oxo-1,2-dihydropyridine-3-carbonitrile. The mixture was stirred at room temperature for 1 hour, heated under reflux for 3 hours, and then concentrated under reduced pressure. To the residue was added 50 ml of water, followed by extraction with 50 ml of chloroform. An aqueous layer was acidified with 10 ml of 10% hydrochloric acid, and then filtered. The filtered crystal was washed with ethyl acetate to obtain 0.38 g of 5-[3-(3-carboxymethoxyphenyl)acryloyl]-6-methyl-2-oxo-1,2-dihydropyridine-3-carboxylic acid [Compound No. (19c)] as a yellow crystal.

¹H-NMR (270 MHz, DMSO-d₆) δ (ppm): 2.59 (s, 3H), 4.75 (s, 2H), 6.95˜7.05 (1H), 7.25˜7.50 (3H), 7.50˜7.80 (3H), 8.85 (s, 1H)

### Examples 2-33

### Synthesis of Compound (Vc) [Compound No. (20c)]

In 5 ml of chloroform were dissolved 0.53 g of 3-acetylchromen-4-one, 0.50 g of 3-(3-hydroxypropoxy)benzaldehyde and 0.32 ml of piperidine, and the solution was heated under reflux for 4 hours and 40 minutes while water was removed with a Soxhlet's extractor filled with molecular sieves. After 40 ml of water was added thereto, the mixture was extracted with 80 ml of chloroform. An organic layer was washed with an aqueous saturated sodium chloride solution, dried over anhydrous sodium sulfate, and then concentrated under reduced pressure. The residue was subjected to silica gel column chromatography to obtain 107 mg of 3-[3-[3-(3-hydroxypropoxy)phenyl]acryloyl]chromen-4-one [Compound No. (20c)] as a light yellow crystal.

¹H-NMR (400 MHz, CDCl₃) δ (ppm): 1.74 (broad s, 1H), 2.04˜2.10 (2H), 3.90 (q, 2H, J=4.9 Hz), 4.19 (t, 2H, J=5.9 Hz), 6.96 (d, 1H, J=7.3 Hz), 7.23˜7.33 (m, 3H), 7.50 (t, 1H, J=8.1 Hz), 7.54 (d, 1H, J=8.3 Hz), 7.74 (dt, 1H, J=1.7, 7.8 Hz), 7.80 (d, 1H, J=15.6 Hz), 8.06 (d, 1H, J=15.6 Hz), 8.33 (dd, 1H, J=1.7, 8.1 Hz), 8.70 (s, 1H)

### Examples 2-34

### Synthesis of Compound (Vc) [Compound No. (21c)]

According to the same manner as that of Example 2-30 except that 753 mg of 3-acetylchromen-4-one was used in place of 3-acetyl-6-hydroxy-2-methyl-1H-pyridin-4-one and 645 mg of 3-(cyanomethoxy)benzaldehyde was used in place of 3-[(methoxycarbonyl)methoxy]benzaldehyde, 67.1 mg of 3-[3-[3-(cyanomethoxy)phenyl]acryloyl]chromen-4-one [Compound No. (21c)] was obtained as a yellow crystal.

¹H-NMR (400 MHz, CDCl₃) δ (ppm): 4.82 (s, 2H), 7.02˜7.04 (1H), 7.30-7.75 (7H), 7.78 (d, 1H, J=15.9 Hz), 8.07 (d, 1H, J=15.9 Hz), 8.31 (1H)

### Examples 2-35

### Synthesis of Compound (Vc) [Compound No. (22c)]

According to the same manner as that of Example 2-34 except that 188 mg of 3-(methoxyacetylamino)benzaldehyde was used in place of 3-(cyanomethoxy)benzaldehyde, 118 mg of 3-[3-[3-(methoxyacetylamino)phenyl]acryloyl]]chromen-4-one [Compound No. (22c)] was obtained as a yellow crystal.

¹H-NMR (400 MHz, CDCl₃) δ (ppm): 3.54 (s, 3H), 4.04 (s, 2H), 7.35˜7.60 (m, 5H), 7.70˜7.85 (m, 4H), 8.06 (d, 1H, J=15.6 Hz), 8.34 (1H), 8.770 (1H)

### Examples 2-36

### Synthesis of Compound (Vc) [Compound No. (23c)]

To a solution of 115 mg of 3-[(2-methoxyethyl)aminocarbonyl]benzaldehyde in 3 ml of ethanol were added 104 mg of 3-acetyl-4(1H)-quinolinone and 13.2 mg of piperidine, and the mixture was heated under reflux for 15 hours and 55 minutes. After cooling, the reaction solution was concentrated. The residue was washed with tetrahydrofuran to obtain 13 mg of 3-[3-[3-[(2-methoxyethyl)aminocarbonyl]phenyl}acryloyl]-4(1H)-quinolinone [Compound No. (23c)] as a light brown crystal.

¹H-NMR (300 MHz, DMSO-d₆) δ (ppm): 3.29 (s, 3H), 3.40˜3.50 (m, 4H), 7.49 (dt, 1H, J=0.9, 7.8 Hz), 7.55 (t, 1H, J=7.8 Hz), 7.69 (d, 1H, J=6.3 Hz), 7.69 (d, 1H, J=15.9 Hz), 7.76 (dt, 1H, J=1.5, 8.4 Hz), 7.86 (d, 1H, J=7.8 Hz), 7.90 (d, 1H, J=7.8 Hz), 8.20 (s, 1H), 8.28 (dd, 1H, J=0.9, 8.1 Hz), 8.38 (d, 1H, J=15.9 Hz), 8.59 (s, 1H), 8.69 (broad, 1H)

### Examples 2-37

### Synthesis of Compound (Vc) [Compound No. (24c)]

To a solution of 103 mg of 3-[(2-methoxyethyl)aminocarbonyl]benzaldehyde in 10 ml of ethanol were added 104 mg of 3-acetyl-1-methyl-4(1H)-quinolinone and 90 mg of potassium hydroxide, and the mixture was stirred at room temperature overnight. Precipitated crystals were filtered, washed with water, and then dried to obtain 74 mg of 3-[3-[3-[(2-methoxyethyl)aminocarbonyl]phenyl}acryloyl]-1-methyl-4(1H)-quinolinone [Compound No. (24c)] as white powder.

¹H-NMR (270 MHz, DMSO-d₆) δ (ppm): 3.27 (s, 3H), 3.43˜3.48 (4H), 4.01 (s, 3H), 7.55 (t, 1H, J=7.6 Hz), 7.57 (t, 1H, J=7.3 Hz), 7.70 (d, 1H, J=15.7 Hz), 7.80 (d, 1H, J=7.8 Hz), 7.83˜7.95 (3H), 8.21 (s, 1H), 8.37 (d, 1H, J=15.9 Hz), 8.37 (d, 1H, J=7.8 Hz), 8.68 (1H), 8.78 (s, 1H)

### Examples 2-38

### Synthesis of Compound (Vc) [Compound No. (25c)]

According to the same manner as that of Example 2-36 except that 360 mg of 3-acetyl-2-methyl-4(1H)-quinolinone was used in place of 3-acetyl-4(1H)-quinolinone, 70 mg of 3-[3-[3-[(2-methoxyethyl)aminocarbonyl]phenyl}acryloyl]-2-methyl-4(1H)-quinolinone [Compound No. (25c)] was obtained as white powder.

¹H-NMR (270 MHz, DMSO-d₆) δ (ppm): 2.45 (s, 3H), 3.26 (s, 3H), 3.45˜3.55 (4H), 7.35˜7.42 (2H), 7.48 (d, 1H, J=18.9 Hz), 7.45˜7.58 (1H), 7.56 (d, 1H, J=16.2 Hz), 7.72 (t, 1H, J=5.4 Hz), 7.82 (d, 1H, J=8.1 Hz), 7.88 (d, 1H, J=8.1 Hz), 8.10˜8.15 (1H), 8.15 (s, 1H), 8.63 (broad, 1H), 12.00 (broad s, 1H)

### Example 2-39

### Synthesis of Compound (Ve) [Compound No. (3e)]

According to the same manner as that of Example 2-30 except that 5.65 g of 3-acetylchromen-2-one was used in place of 3-acetyl-6-hydroxy-2-methyl-1-H-pyridin-4-one and 4.22 g of 3-chlorobenzaldehyde was used in place of 3-[(methoxycarbonyl)methoxy]benzaldehyde, 0.30 g of 3-[3-(3-chlorophenyl)acryloyl]chromen-2-one [Compound No. (3e)] was obtained as a light yellow crystal.

¹H-NMR (300 MHz, CDCl₃) δ (ppm): 7.32˜7.45 (4H), 7.52˜7.58 (1H), 7.64˜7.72 (3H), 7.77 (d, 1H, J=15.7 Hz), 7.95 (d, 1H, J=15.7 Hz), 8.60 (1H)

### Example 3

### Preparation of a Plasmid Having a Reporter Gene Linked to a Transcription Regulatory Region for a Type I Collagen Gene

1×10⁸ cells of a normal human fetal skin fibroblast (Clontech, catalogue No. CC-2509) were cultured at 37° C. overnight under 5% CO₂ atmosphere. After the cultured cells were washed with a sodium phosphate buffer (hereinafter, referred to as PBS) twice, 3 ml of PBS was added thereto and the cells were scraped away the wall of a vessel using a cell scraper (Nalgen, catalogue No. 179693). The scraped cells were collected by centrifugation (1,500 rpm, 4° C., 15 min), and these were suspended in 20 ml of PBS and centrifuged again. To the resulting precipitates were added 11 ml of Solution 2 and 4.8 μl of pronase of DNA Extraction Kit (Stratagene, catalogue No. 200600). After shaken at 60° C. for 1 hour, the resulting mixture was allowed to stand in ice for 10 minutes. Then, 4 ml of Solution 3 of the kit was added to the mixture. After mixed, the mixture was allowed to stand in ice for 5 minutes and then centrifuged (3,000 rpm, 4° C., 15 min) to recover a supernatant. To the recovered supernatant was added 2 μl of RNase per 1 ml of the supernatant and the mixture was allowed to stand at 37° C. for 15 minutes. To the mixture was added 2-fold volume of ethanol. After mixed, a white thread-like substance (genomic DNA) appeared and the substance was recovered. The recovered genomic DNA was washed with 70% ethanol and then air-dried. The air-dried genomic DNA was dissolved in 500 μl of 10 mM Tris-HCl, 1 mM EDTA (pH 8.0) (hereinafter, referred to as TE).

The resulting genomic DNA-dissolved solution (genomic DNA 1 μg corresponding amount) was mixed with each 1 μl of an oligonucleotide consisting of a base sequence represented by SEQ ID No.: 1 (SEQ ID No. 1: an oligonucleotide primer designed to amplify a collagen promoter DNA: ccaagctagc gaaattatct tttctttcat ag 32) and an oligonucleotide (10 μmol/μl) consisting of a base sequence represented by SEQ ID No.:2 (SEQ ID No. 2: an oligonucleotide primer designed to amplify a collagen promoter DNA: ccaaaagctt gcagtcgtgg ccagtacc 28), 29 μl of distilled water, 5 μl of a buffer attached to TaKaRa LA Taq (TAKARA SHUZO Co., Ltd., catalogue No. RR002A), 5 μl of a Mg²⁺ solution, 5 μl of a dNTP mixture and 0.5 μl of TaKaRa LA Taq (TAKARA SHUZO Co., Ltd., catalogue No. RR002A). After the resulting mixed solution was incubated at 94° C. for 5 minutes, the mixed solution was subjected to 30 cycles, in which one cycle consists of incubation at 94° C. for 1 minute, at 60° C. for 1 minute and then at 72° C. for 1 minute. The mixed solution was electrophoresed on a 2% agarose gel to recover about 0.5 kb of a DNA. The recovered DNA was treated with phenol/chloroform and then precipitated with ethanol to recover the DNA. The resulting DNA was dissolved in ultrapure water. To this solution were added 2.5 μl of NheI and 2.5 μl of HindIII, and then incubated at 37° C. for 3 hours. Then, the solution was electrophoresed on a 2% agarose gel to recover about 3.5 kb of a DNA. The recovered DNA was precipitated with ethanol to recover again the DNA (hereinafter, referred to as the collagen promoter DNA).

On the other hand, the vector pGL3 (Promega, catalogue No. E1751) having the nucleotide sequence encoding firefly luciferase was digested with NheI and HindIII, and then subjected to agarose gel electrophoresis as described above to recover about 5 kb of a DNA. The recovered DNA was precipitated with ethanol to recover the DNA again. To the recovered DNA were added 44 μl of distilled water, 5 μl of Buffer attached to Alkaline Phosphatase (TAKARA SHUZO, catalogue No. 2120A) and 1 μl of Alkaline Phosphatase (TAKARA SHUZO, catalogue No. 2120A). The mixed solution was incubated at 65° C. for 30 minutes. Then, the mixed solution was treated with phenol/chloroform twice, and precipitated with ethanol to recover the DNA (hereinafter referred to as the Luc vector DNA). Then, after about 20 ng of the collagen promoter DNA and about 20 ng of the Luc vector DNA were mixed, the same amount of a DNA Ligation kit Ver2 enzyme solution was added and this was incubated for 1 day at 16° C. To the mixed solution was added Escherichia coli 5Hdα (TOYOBO, catalogue No. DNA-903), this was allowed to stand in ice for 30 minutes, and then incubated at 42° C. for 45 seconds. The resulting Escherichia coli was seeded on a L_{B} plate containing 50 μg/ml ampicillin sodium (Nacalai, catalogue No. 027-39), and this was allowed to stand at 37° C. for 1 day. A single colony appeared and the colony was cultured in 2 ml of a LB medium containing 50 μg/ml ampicillin at 37° C. for 12 hours. From the resulting culture solution, a plasmid DNA was prepared using AUTOMATIC DNA ISOLATION SYSTEM PI-50 (KURABO). The nucleotide sequence of the prepared plasmid DNA was analyzed with a DNA sequencer. As a result, it was confirmed that the plasmid (hereinafter, referred to as COL-Luc) had a nucleotide sequence comprising a nucleotide sequence encoding the amino acid sequence of firefly luciferase as a reporter gene linked downstream of the nucleotide sequence −3500 to +57 (the transcription initiation point is +1) of a transcription regulatory region for a human-derived Type I collagen α2 chain gene.

### Example 4

### Measurement of the Ability of a Test Compound to Regulate Transcription of a Type I Collagen Gene Using the Expression Level of a Report Gene as an Index

1×10⁶ cells of a normal human fetal skin fibroblast were seeded on a 100 mm dish and cultured at 37° C. overnight under 5% CO₂ atmosphere in a Dulbecco's-MEM (Nissui Seiyaku, catalogue No. 05919) medium containing 10(v/v) % heat-inactivated bovine fetal serum (hereinafter, referred to as FBS; Gibco, catalogue No. 21140-079) (hereinafter, this medium is referred to as D-MEM(+)). Then, the medium was replaced with a Dulbecco's-MEM medium not containing FBS (hereinafter, this medium is referred to as D-MEM(−)).

To 300 μl of D-MEM(−) were added 5 μg of COL-Luc and 5 μg of pCMV-β-gal (Invitrogen, catalogue No. 10586-014), and the resulting mixed solution was allowed to stand at room temperature for 5 minutes (solution 1). To 300 μl of D-MEM(−) was added 20 μl of Lipofectine (Gibco, catalogue No. 18292-011), and the resulting mixed solution was allowed to stand at room temperature for 45 minutes (solution 2). Then, the solution 1 and the solution 2 were mixed. After the mixture was allowed to stand at room temperature for 10 minutes, 5.4 ml of D-MEM(−) was added to thereto, followed by mixing. The mixed solution was added to the normal human fetal skin fibroblasts, and the cells were cultured at 37° C. under 5% CO₂ atmosphere. After 6 hours, the culture supernatant was removed from the dish, and the cells were washed with PBS twice. To the dish was added 1 ml of PBS containing 0.25% trypsin, and the cells were scraped off the dish. To the scraped cells was added D-MEM(+), and these were mixed well. The mixture was dispensed into a 12-well plate at 1 ml per well, and the plate was incubated at 37° C. overnight under 5% CO₂ atmosphere. On the next day, each well was washed with D-MEM(−) twice, and this was replaced with 1 ml of a Dulbecco's-MEM medium containing 0.1% FBS (hereinafter, this medium is referred to as D-MEM (0.1%)).

To the thus cultured cells was added 10 μL of a 1 mM solution of the present compound represented by the compound number (20a), (56a), (124a), (11c) or (16c) in 10% dimethyl sulfoxide (hereinafter, referred to as DMSO) (the present compound final concentration: 10 μM, DMSO final concentration: 0.1%). As a control, only 10 μl of DMSO was added. In addition, to the cultured cells was added 10 μL of a 0.3 mM solution of the present compound represented by the compound number (19a), (29a), (31a), (32a), (36a), (40a), (42a), (1b), (10b), (13b) or (24c) in 10% DMSO (the present compound final concentration: 3 μM, DMSO final concentration: 0.1%). In addition, to the cultured cells was added 10 μL of a 0.1 mM solution of the present compound represented by the compound number (5b), (16b) or (23c) in 10% DMSO (the present compound final concentration: 1 μM, DMSO final concentration: 0.1%).

After one hour, 10 μl of a 0.5 μg/ml aqueous solution of TGF-β (Pepro Tech) or distilled water was added to the well, and the plate was further incubated at 37° C. for 40 hours under 5% CO₂ atmosphere. After the incubated cells were washed with PBS twice, 200 μl of a cell lysing agent (Toyo Inc., catalogue No. PD10) was added thereto and the cells were scraped. The scraped cells were recovered as a cell suspension, and the suspension was centrifuged (15,000 rpm, 4° C., 5 min) to recover a supernatant. The recovered supernatant was transferred to a 96-well plate at 50 μl per well, and then 50 μl of a Luc assay solution (20 mM Tricine (pH 7.8), 2.67 mM MgSO₄, 0.1 mM EDTA, 33.3 mM DTT, 270 μM Coenzyme A, 530 μM ATP, 470 μM Luciferin) was automatically dispensed into the plate using MICROLUMAT LB96P (manufactured by EG&G BERTHOLD). Luminescence in each well was measured (Delay: 1.6 second, Meas. Interval: 20 second).

On the other hand, 50 μl of the recovered supernatant or the cell lysing agent was added to 50 μl of a β-gal substrate solution (5.8 mM o-nitrophenyl-beta-D-galactopyranoside, 1 mM MgCl₂, 45 mM 2-mercaptoethanol) which had been dispensed into a 96-well plate in advance, and the plate was incubated at 37° C. for 2 hours. Then, an absorbance in each well was measured using a microplate reader at 420 nm. Based on the value obtained, transcription activity was calculated according to the following equation.

Transcription activity=[luminescence amount(supernatant-added section)−luminescence amount(cell lysing agent-added section)]/[420 nm absorbance(supernatant-added section)−420 nm absorbance(cell lysing agent-added section)]

Then, based on the calculated transcription activity, an inhibitory effect of a test compound on the ability of TGF-β to promote transcription of a Type I collagen gene was calculated as an inhibition percentage according to the following equation:

Inhibition percentage=[transcription activity(DMSO and TGF-β-added test section)−transcription activity(compound and TGF-β-added test section)]/[transcription activity(DMSO and TGF-β-added test section)−transcription activity(DMSO and TGF-β non-added test section)]×100

The inhibition percentages of the present compounds represented by the compound numbers (20a), (56a), (124a), (11c) and (16c) at the present compound final concentration of 10 μM, the present compounds represented by the compound numbers (19a), (29a), (31a), (32a), (36a), (40a), (42a), (1b), (10b), (13b) and (24c) at the present compound final concentration of 3 μM, and the present compounds represented by the compound numbers (5b), (16b) and (23c) at the present compound final concentration of 1 μM were 70 or more. It was found that the present compounds can inhibit the ability of TGF-β to promote transcription of a Type I collagen gene, and then can suppress transcription of a Type I collagen gene.

### Example 5

### Measurement of the Ability of a Test Compound to Suppress TGF-β Using the Expression Level of Fibronectin as an Index

5×10³ cells/well of a normal human skin fibroblast (Clonetics, catalogue No. CC-2509) were seeded on a 96-well plate (BECTON DICKINSON, catalogue No. 35-3075), and then cultured in an incubator at 37° C. and 5% CO₂ overnight. On the next day, the medium was exchanged with 0.1 ml of a D-MEM medium (Nissui Pharmaceutical Co., Ltd., code No. 05919) containing 0.1% bovine fetal serum. After 1 hour, to the well was added the present compound represented by the compound number (31a), (10b) or (13c) at the final concentration of 10 μM. After the cells were cultured for 1 hour, 5 ng/ml (final concentration) of TGF-β (Peprotech, catalogue No. 100-21R) was added, followed by further culturing for 26 hours. After the cells were washed with a phosphate buffer twice, a total RNA was isolated using SV96 Total RNA Isolation System (Promega, catalogue No. Z3505). To 5 ml of the isolated total RNA were added 1 μl of 20 μM oligo dT and 4 μl of RNase-free distilled water, incubated at 65° C. for 5 minutes, and immediately cooled with an ice. To 10 μl of the solution were added 4 μl of 5× buffer, 2.4 μl of MgCl₂, 1 μl of 10 mM dNTP, 1 μl of RNasin, 1 μl of Improm II and 0.6 μl of RNase-free distilled water (these are all available from Promega), and the mixture was subjected to a reverse transcription reaction under the condition of 25° C. for 5 minutes, 42° C. for 1 hour and 70° C. for 15 minutes.

To 5 μl of a reverse transcription reaction solution were added each 1 μl of each 20 μmol/μl of primers represented by SEQ ID No. 3 (SEQ ID No. 3: an oligonucleotide designed as a PCR primer in order to detect a DNA of a fibronectin gene: tcgccatcag tagaaggtag ca 22) and SEQ ID No. 4 (SEQ ID No. 4: an oligonucleotide designed as a PCR primer in order to detect a DNA of a fibronectin gene: tatactgaac accaggttgc aagtc 25), and 1.25 μL of a probe for detecting a DNA of a fibronectin gene represented by SEQ ID No. 5 (SEQ ID NO. 5: an oligonucleotide designed as a probe in order to detect a DNA of a fibronectin gene: ctcaaccttc ctgaaactgc aaactccgtc 30) or 1.25 μl of Human GAPDH primer probe (Applied Biosystems, catalogue No. 4310884E). Further, 12.5 μl of TaqMan Universal PCR Master Mix (Applied Biosystems, catalogue No. 4304437) was added, and the mixture was adjusted to 50 μl with sterilized water, and then mixed in a well of Optical 96-Well Reaction Plate (Applied Biosystems, catalogue No. N801-0560). As a standard, a total RNA was prepared from the cells to which only TGF-β had been added, and 250, 125, 62.5, 31.25, 15.625 or 7.8125 ng of the total RNA was subjected to a reverse transcription reaction to obtain a cDNA solution. Then, PCR was performed under the condition of 1 cycle of 50° C. for 5 minutes and 40 cycles of 95° C. for 15 seconds and 60° C. for 1 minute using Gene Amp 7900 (Applied Biosystems). For quantitation, respective standard lines for fibronectin and GAPDH were made. Then the fibronectin amount and the GAPDH amount were calculated, and the transcription amount was calculated according to the following equation.

Fibronectin transcription amount=fibronectin amount/GAPDH amount

Inhibition percentage=[transcription amount (DMSO and TGF-β-added test section)−transcription amount (compound and TGF-β-added test section)]/[transcription amount (DMSO and TGF-β-added test section)−transcription amount (DMSO and TGF-β non-added test section)]×100

The inhibition percentages of the present compounds represented by the compound numbers (31a), (10b) and (13c) were 70% or more.

It was confirmed that the present compound suppresses the transcription amount of a fibronectin gene of a skin fibroblast promoted by TGF-β.

### INDUSTRIAL APPLICABILITY

According to the present invention, it is possible to develop and provide a composition which decreases expression of an extracellular matrix gene in a tissue to induce a reduction in accumulation of an extracellular matrix and thereby improves tissue fibrosis (i.e. an extracellular matrix accumulation-suppressing agent, a fibrosing disease-treating agent, or heart failure treating agent).

### Sequence Listing Free Text

### SEQ ID NO: 1

Oligonucleotide primer designed for amplifying a collagen promoter DNA

### SEQ ID NO: 2

Oligonucleotide primer designed for amplifying a collagen promoter DNA

### SEQ ID NO: 3

Oligonucleotide primer designed for detecting a fibronectin DNA

### SEQ ID NO: 4

Oligonucleotide primer designed for detecting a fibronectin DNA

### SEQ ID NO: 5

Oligonucleotide primer designed for detecting a fibronectin DNA

1. A composition for suppressing transcription of an extracellular matrix gene which comprises an inert carrier and a cinnamoyl compound represented by the formula (I): 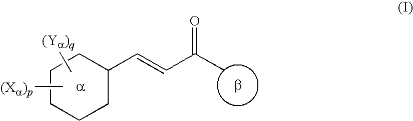
wherein,
I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;
(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a Rₑ—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ— CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y₀ group: a M_{b0}-R_{d}— group, wherein M_{b0} represents a M_{c0}- group
[wherein M_{c0} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by 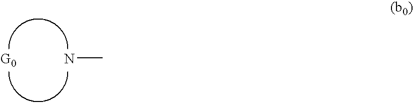
(wherein G₀ forms an optionally substituted, saturated or unsaturated, nonaromatic 5 to 14-membered hydrocarbon ring or heterocyclic ring), a (c₀)- group represented by 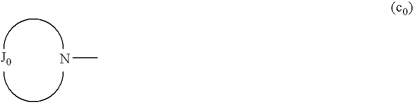
(wherein, J₀ forms a 5 to 7-membered aromatic ring optionally containing a nitrogen atom), a (d₀)- group represented by 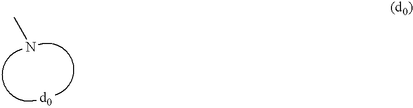
[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B1 represents an oxy group, a thio group, a sulfinyl group or a sulfonyl group), a C3-C10 alkenyl group, or a C3-C10 alkynyl group}, a sulfinyl group, or a sulfonyl group], or a (e₀)- group represented by 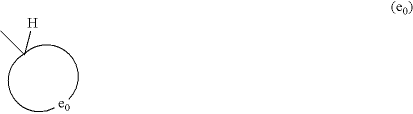
{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}; and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;
(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and
II. β represents
a group represented by formula (I-1): 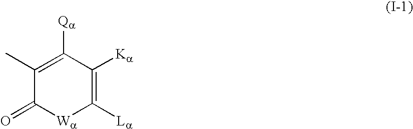
wherein,
(1) Q_{α} represents an optionally substituted hydroxyl group, or an optionally substituted amino group,
(2) W_{α} represents an oxygen atom or a —NT_{α}- group (wherein T_{α} represents a hydrogen atom, or a substituent on the nitrogen atom),
(3) K_{α} and L_{α} form a —V_{α}═V_{α}′—V_{α}″═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (I-2): 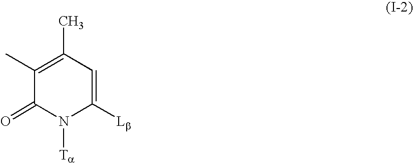
wherein T_{α} is as defined above, and L_{β} represents a hydroxyl group or a methyl group;
a group represented by formula (I-3): 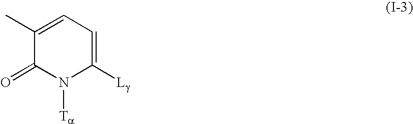
wherein T_{α} is as defined above, and L_{γ} represents a C1-C10 alkyl group;
a group represented by formula (I-4): 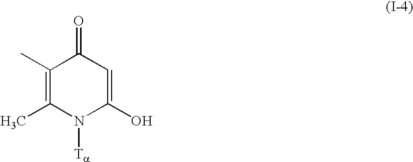
wherein T_{α} is as defined above;
a group represented by formula (I-5): 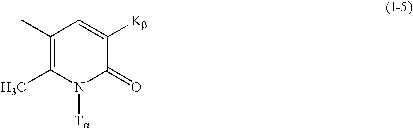
wherein T_{α} is as defined above, and K_{β} represents a cyano group or a UOCO— group (wherein U represents a hydrogen atom or a C1-C10 alkyl group);
a group represented by formula (I-6): 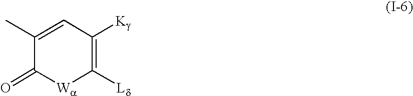
wherein W_{α} is as defined above, and K_{γ} and L_{δ} form an optionally substituted C3-C10 alkylene group or an optionally substituted C4-C10 alkenylene group;
a group represented by formula (I-7): 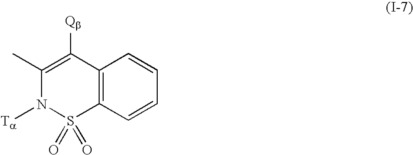
wherein T_{α} is as defined above, and Q_{β} represents an optionally substituted hydroxyl group; or
a group represented by formula (I-8): 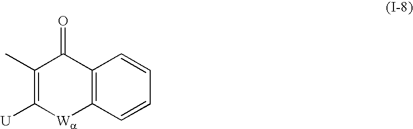
wherein U and W_{α} are as defined above;
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

2. A cinnamoyl compound represented by the formula (II): 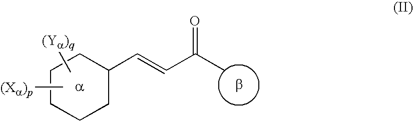
wherein,
I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;
(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a Rₑ—Bₐ—R_{d}— group (wherein Rₑ represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ— CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y₀ group: a M_{b0}-R_{d}— group, wherein M_{b0} represents a M_{c0}- group
[wherein M_{c0} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by 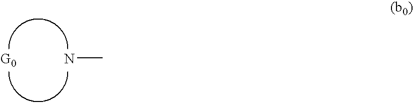
(wherein G₀ forms an optionally substituted, saturated or unsaturated, nonaromatic 5 to 14-membered hydrocarbon ring or heterocyclic ring), a (c₀)- group represented by 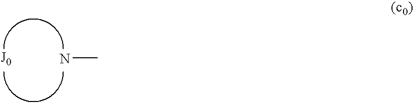
(wherein, J₀ forms a 5 to 7-membered aromatic ring optionally containing a nitrogen atom), a (d₀)- group represented by 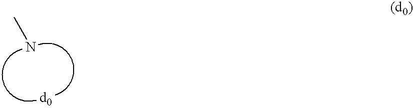
[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group), a sulfinyl group, or a sulfonyl group], or a (e₀)- group represented by 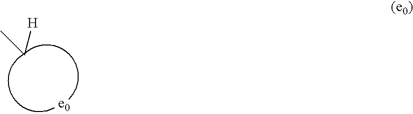
{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group); and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and R_{d} are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;
(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and
II. β represents
a group represented by formula (II-1): 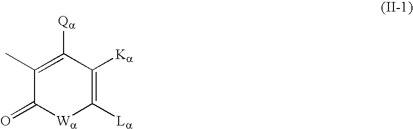
wherein,
(1) Q_{α} represents an optionally substituted hydroxyl group, or an optionally substituted amino group,
(2) W_{α} represents an oxygen atom or a —NT_{α}— group (wherein T_{α} represents a hydrogen atom, or a substituent on the nitrogen atom),
(3) K_{α} and L_{α} form a —V_{α}═V_{α}′—V_{α}″═V_{α}′″— group (wherein V_{α}, V_{α}′, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (II-2): 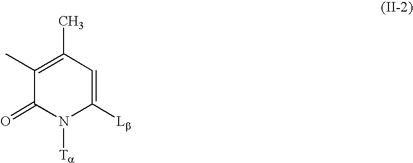
wherein T_{α} is as defined above, and L_{β} represents a hydroxyl group or a methyl group;
a group represented by formula (II-3): 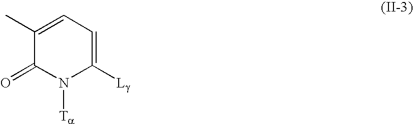
wherein T_{α} is as defined above, and L_{γ} represents a C1-C10 alkyl group;
a group represented by formula (II-4): 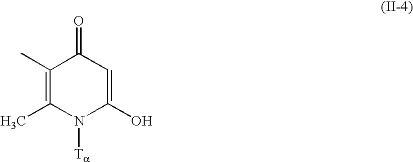
wherein T_{α} is as defined above;
a group represented by formula (II-5): 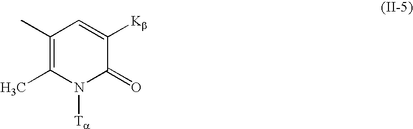
wherein T_{α} is as defined above, and K_{β} represents a cyano group or a UOCO— group (wherein U represents a hydrogen atom or a C1-C10 alkyl group);
a group represented by formula (II-6): 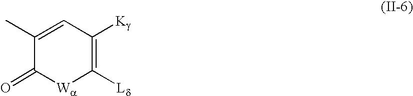
wherein W_{α} is as defined above, and K_{γ} and L_{δ} form an optionally substituted C3-C10 alkylene group or an optionally substituted C4-C10 alkenylene group;
a group represented by formula (II-7): 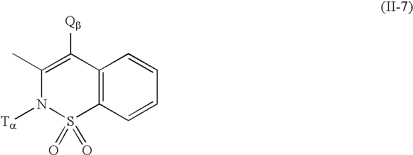
wherein T_{α} is as defined above, and Q_{β} represents an optionally substituted hydroxyl group; or
a group represented by formula (II-8): 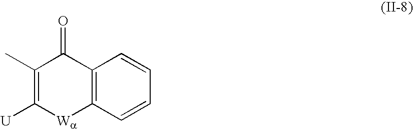
wherein U and W, are as defined above;
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

3. A cinnamoyl compound represented by the formula (III): 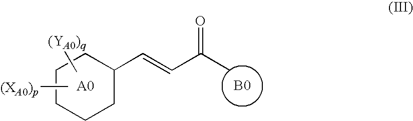
wherein
I. A0 represents a benzene ring or pyridine ring;
II. in (X_{A0})ₚ, X_{A0} is a substituent on a carbon atom and represents a group included in any group of the following A₀ group to the N₀ group, p represents 0, 1, 2, 3, 4 or 5, and when p is not lees than 2, X_{A0}s are the same or different;
(1) the A₀ group:
a D₁-R₄— group, wherein D₁ represents a (R₁—(O)ₖ—)A₁N—(O)_{k′}— group [wherein R₁ represents a hydrogen atom, or a C1-C10 alkyl group, or a C2-C10 alkyl group substituted with a halogen atom or a R₂—B₁-group (wherein R₂ represents a C1-C10 alkyl group, a C3-C10 alkenyl group or a C3-C10 alkynyl group, and B₁ represents an oxy group, a thio group, a sulfinyl group or a sulfonyl group), or a C3-C10 alkenyl group, or a C3-C10 alkynyl group, k represents 0 or 1, A₁ represents a R₃—(CHR₀)ₘ—(B₂—B₃)_{m′}— group {wherein R₃ represents a hydrogen atom, or a C1-C10 alkyl group optionally substituted with a halogen atom or a R₂—B₁— group (wherein R₂ and B₁ are as defined above), or a C2-C10 alkenyl group, or a C2-C10 alkynyl group, Rₒ represents a hydrogen atom, a C1-C10 alkyl group or a C2-C10 haloalkyl group, m represents 0 or 1, B₂ represents a single bond, an oxy group, a thio group or a —N((O)ₙR₁′)— group (wherein R₁′ is the same as or different from R₁, and has the same meaning as R₁ has, and n represents 0 or 1), B₃ represents a carbonyl group, a thiocarbonyl group or a sulfonyl group, m′ represents 0 or 1, and B₃ is not a sulfonyl group when m is 0 and R₃ is a hydrogen atom}, and k′ represents 0 or 1], and R₄ represents a C1-C10 alkylene group, provided that a R₀′R₀″N—R₄— group (wherein R₀′ and R₀″ are the same as or different from Rₒ and have the same meaning as Rₒ has, and R₄ is as defined above) is excluded,
a D₂-R₄— group, wherein D₂ represents a cyano group, a R₁R₁′NC(═N—(O)ₙ-A₁)— group (wherein R₁, R₁′, n and A₁ are as defined above), an A₁N═C(—OR₂)— group (wherein A₁ and R₂ are as defined above) or a NH₂—CS— group, and R₄ is as defined above,
a D₃-R₄— group, wherein D₃ represents a nitro group or a R₁OSO₂— group (wherein R₁ is as defined above), and R₄ is as defined above, and
a R₁OSO₂— group, wherein R₁ is as defined above;
(2) the B₀ group: an (a₀)- group represented by 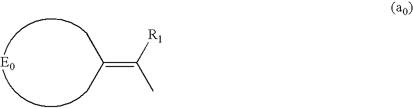
wherein E₀ forms an optionally substituted, saturated or unsaturated, aromatic or nonaromatic 5 to 14-membered hydrocarbon ring or heterocyclic ring, and R₁ is as defined above;
(3) the C₀ group: a C2-C10 alkenyl group substituted with a halogen atom, a R₂—B₁— group (wherein R₂ and B₁ are as defined above), a D₄-R₄— group [wherein D₄ represents a hydroxyl group or an A₁-O— group (wherein A₁ is as defined above), and R₄ is as defined above], a D₅- group [wherein D₅ represents a O═C(R₃)— group (wherein R₃ is as defined above), an A₁-(O)ₙ—N═C(R₃)— group (wherein A₁, n and R₃ are as defined above), a R₁—B₀—CO—R₄—(O)ₙ—N═C(R₃)— group {wherein R₁, R₄, n and R₃ are as defined above, and B₀ represents an oxy group, a thio group or a —N((O)ₘR₁′)— group (wherein R₁′ and m are as defined above)}, a D₂-R₄—(O)ₙ—N═C(R₃)— group (wherein D₂, R₄, n and R₃ are as defined above) or a R₁A₁N—N═C(R₃)— group (wherein R₁, A₁ and R₃ are as defined above)], a R₁A₁N—O—R₄— group (wherein R₁, A₁ and R₄ are as defined above), a R₁(A₁-(O)ₙ—)N— group (wherein R₁, A₁ and n are as defined above), a D₂- group (wherein D₂ is as defined above) or a D₃- group (wherein D₃ is as defined above);
(4) the D₀ group: a C2-C10 alkynyl group substituted with a (b₀)-R₄— group (in (b₀) 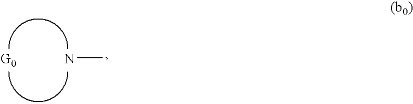
G₀ forms an optionally substituted, saturated or unsaturated, nonaromatic 5 to 14-membered hydrocarbon ring or heterocyclic ring), a (c₀)-R₄— group (in (c₀) 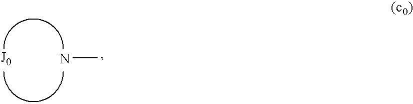
J₀ forms an aromatic 5 to 7-membered ring optionally containing a nitrogen atom and R₄ is as defined above), a halogen atom, a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above) or a D₃-R₄— group (wherein D₃ and R₄ are as defined above);
(5) the E₀ group: an A₂-CO—R₅— group, provided that R₅ is not a vinylene group when A₂ is a hydroxyl group
[wherein A₂ represents
(i) an A₃-B₄— group
wherein A₃ represents a hydrogen atom, or a C1-C10 alkyl group, or a C2-C10 haloalkyl group, or a C2-C10 alkenyl group optionally substituted with a halogen atom, or a C3-C10 alkynyl group optionally substituted with a halogen atom, or a RₐO—(R₄)ₘ— group (wherein Rₐ₀ represents an optionally substituted 5 to 7-membered aryl group or heteroaryl group, and R₄ and m are as defined above), or a C1-C10 alkyl group substituted with a (b₀)-R₄— group (wherein (b₀) and R₄ are as defined above), a (c₀)-R₄— group (wherein (c₀) and R₄ are as defined above), a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above), a D₃-R₄— group (wherein D₃ and R₄ are as defined above) or an A₄-SO₂—R₄— group {wherein A₄ represents a (b₀)- group (wherein (b₀) is as defined above), a (c₀)- group (wherein (c₀) is as defined above) or a R₁R₁′N— group (wherein R₁ and R₁′ are as defined above), and R₄ is as defined above}, and
B₄ represents an oxy group, a thio group or a —N((O)ₘR₁)— group (wherein R₁ and m are as defined above), provided that A₃ is not a hydrogen atom when B₄ is a thio group;
(ii) a R₁—B₄—CO—R₄—B₄′— group (wherein R₁, B₄ and R₄ are as defined above, B₄′ is the same as or different from B₄ and has the same meaning as B₄ has, provided that R₂ is not a hydrogen atom when B₄ is a thio group) or a D₂-R₄—B₄— group (wherein D₂, R₄ and B₄ are as defined above);
(iii) a R₂—SO₂—NR₁— group (wherein R₂ is as defined above, provided that a hydrogen atom is excluded, and R₁ is as defined above);
(iv) a (b₀)- group, wherein (b₀) is as defined above;
(v) a (c₀)- group, wherein (c₀) is as defined above; or
(vi) a R₁A₁N—NR₁′— group, wherein R₁, A₁ and R₁′ are as defined above; and
R₅ represents a C2-C10 alkenylene group optionally substituted with a halogen atom or a C2-C10 alkynylene group];
(6) the F₀ group: an A₅-B₅—R₆— group, wherein
A₅ represents a C2-C10 alkyl group substituted with a D₄- group (wherein D₄ is as defined above), a D₁- group (wherein D₁ is as defined above), a D₃- group (wherein D₃ is as defined above) or an A₄-SO₂— group (wherein A₄ is as defined above), or a C1-C10 alkyl group substituted with a R₂—B₁— group (wherein R₂ and B₁ are as defined above), a D₂- group (wherein D₂ is as defined above), a D₅- group (wherein D₅ is as defined above) or an A₂-CO— group (wherein A₂ is as defined above), and
B₅ represents a B₁— group (wherein B₁ is as defined above) or a —NA₁- group (wherein A₁ is as defined above) and
R₆ represent a single bond or a C1-C10 alkylene group;
(7) the G₀ group: an A₆-B₅—R₆— group wherein
A₆ represents an (a₀)-R₄— group (wherein (a₀) and R₄ are as defined above), or a C2-C10 alkenyl group, or a C2-C10 alkynyl group, or a C2-C10 alkenyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as defined above), or a C2-C10 alkynyl group substituted with a halogen atom, a R₂—B₁— group (wherein R₂ and B₁ are as defined above), a D₅- group (wherein D₅ is as defined above), D₂- group (wherein D₂ is as defined above) or an A₂-CO— group (wherein A₂ is as defined above), or a C3-C10 alkenyl group substituted with a (b₀)- group (wherein (b₀) is as defined above), a (c₀)- group (wherein (c₀) is as defined above), a D₄- group (wherein D₄ is as defined above), a D₁- group (wherein D₁ is as defined above) or a D₃- group (wherein D₃ is as defined above), or a C3-C10 alkynyl group substituted with a D₄- group (wherein D₄ is as defined above), a D₁- group (wherein D₁ is as defined above) or a D₃- group (wherein D₃ is as defined above), and
B₅ and R₆ are as defined above;
(8) the H₀ group:
a D₂-N(—(O)ₙ-A₁)-R₆— group (wherein D₂, n, A₁ and R₆ are as defined above),
a D₂- group (wherein D₂ is as defined above, provided that a cyano group is excluded),
a R₁(R₁′(O)ₙ)N—CR₁ ″═N—R₆— group (wherein R₁, R₁′, n and R₆ are as defined above, R₁ ″ is the same as or different from R₁ and has the same meaning as R₁ has),
a R₁—(O)ₙ—N═CR₁′—NR₂—R₆— group (wherein R₁, n, R₁′, R₂ and R₆ are as defined above),
a R₂—B₃—NR₁—CO—NR₁′—R₆— group (wherein R₂, B₃, R₁, R₁′ and R₆ are as defined above),
a D₂-CO—NR₁—R₆— group (wherein D₂, R₁ and R₆ are as defined above), and
an A₂-COCO—NR₁—R₆— group (wherein A₂, R₁ and R₆ are as defined above);
(9) the I₀ group:
an A₇-B₆—N((O)ₙR₁)—R₆— group [wherein A₇ represents a C2-C10 alkenyl group optionally substituted with a halogen atom, or a C2-C10 alkynyl group, or a C3-C10 haloalkynyl group, or a R₂—B₁—R₄— group (wherein R₂, B₁ and R₄ are as defined above), or a D₄-R₄— group (wherein D₄ and R₄ are as defined above), or a D₅-R₄— group (wherein D₅ and R₄ are as defined above), or a D₁-R₄— group (wherein D₁ and R₄ are as defined above), or a (b₀)-R₄— group (wherein (b₀) and R₄ are as defined above), or a (c₀)-R₄— group (wherein (c₀) and R₄ are as defined above), or a D₂-R₄— group (wherein D₂ and R₄ are as defined above), or a D₃-R₄— group (wherein D₃ and R₄ are as defined above), or an A₄-SO₂—R₄— group (wherein A₄ and R₄ are as defined above), or an A₂-CO—R₄— group (wherein A₂ and R₄ are as defined above), B₆ represents a carbonyl group or a thiocarbonyl group, and n, R₁ and R₆ are as defined above],
an A₈-CS—N((O)ₙR₁)—R₆— group [wherein A₈ represents a hydrogen atom or a C1-C10 alkyl group optionally substituted with a halogen atom, and n, R₁ and R₄ are as defined above],
an A₇′-B₂′—B₃—N((O)ₙR₁)—R₆— group [wherein A₇′ represents a C3-C10 alkenyl group optionally substituted with a halogen atom, or a C3-C10 alkynyl group optionally substituted with a halogen atom, or a R₂—B₁—R₄′— group (wherein R₂ and B₁ are as defined above, and R₄′ represents a C2-C10 alkylene group), or a D₄-R₄′— group (wherein D₄ and R₄′ are as defined above), or a D₁-R₄′— group (wherein D₁ and R₄′ are as defined above), or a (b₀)-R₄′— group (wherein (b₀) and R₄′ are as defined above), or a (c₀)-R₄′— group (wherein (c₀) and R₄′ are as defined above), or a D₂-R₄— group (wherein D₂ and R₄ are as defined above), or a D₃-R₄′— group (wherein D₃ and R₄′ are as defined above), or an A₂-CO—R₄— group (wherein A₂ and R₄ are as defined above), B₂′ represents an oxy group, a thio group or a —N((O)ₙ.R₁′)— group (wherein n′ is the same as or different from n and has the same meaning as n has, and R₁′ is as defined above), and B₃, n, R₁ and R₆ are as defined above],
an A₈′-B₂′—CS—N((O)ₙR₁′)—R₆— group [wherein A₈′ represents a C1-C10 alkyl group or a C2-C10 haloalkyl group, B₂′ is as defined above, and n, R₁ and R₆ are as defined above],
an A₈′-S—B₃′—N((O)ₙR₁′)—R₆— group [wherein A₈′, n, R₁ and R₆ are as defined above, and B₃′ represents a carbonyl group or a sulfonyl group], and
an A₇″-SO₂—N((O)ₙR₁′)—R₆— group [wherein A₇″ represents a C2-C10 alkenyl group, or a C3-C10 alkenyl group substituted with a halogen atom, or a C3-C10 alkynyl group optionally substituted with a halogen atom, or a R₂—B₁—R₄′— group (wherein R₂, B₁ and R₄′ are as defined above), or a D₄-R₄′— group (wherein D₄ and R₄′ are as defined above), or a D₅-R₄— group (wherein D₅ and R₄ are as defined above), or a D₁-R₄′— group (wherein D₁ and R₄′ are as defined above), or a (b₀)-R₄′— group (wherein (b₀) and R₄′ are as defined above), or a (c₀)-R₄′— group (wherein (c₀) and R₄′ are as defined above), or a D₂-R₄— group (wherein D₂ and R₄ are as defined above), or a NO₂—R₄— group (wherein R₄ is as defined above), or an A₂-CO—R₄— group (wherein A₂ and R₄ are as defined above), and n, R₁ and R₆ are as defined above];
(10) the J₀ group:
an A₇-CO— group (wherein A₇ is as defined above),
an A₉-CS— group (wherein A₉ represents A₇ or A₈),
an A₉′(O)ₘN═C(A₉)- group (wherein A₉′ represents A₇′ or A₈′, and m and A₉ are as defined above),
a D₂-CO— group (wherein D₂ is as defined above),
an A₂-COCO— group (wherein A₂ is as defined above),
an A₉-CO—B₁′—R₆— group (wherein A₉ and R₆ are as defined above, and B₁′ represents an oxy group or a thio group, provided that A₉ is not A₈ when B₁′ is an oxy group),
an A₉-CS—B₁′—R₆— group (wherein A₉, B₁′ and R₆ are as defined above),
an A₇″-SO₂—B₁′—R₆— group (wherein A₇″, B₁′ and R₆ are as defined above),
an A₈-SO₂—B 1′-R₆— group (wherein A₈, B₁′ and R₆ are as defined above, provided that A₈ is not a hydrogen atom),
an A₉′-B₂′—B₃—B₁′—R₆— group (wherein A₉′, B₂′, B₃, B₁′ and R₆ are as defined above), and
a C2-C10 alkenyl group substituted with a (b₀)- group (wherein (b₀) is as defined above) or a (c₀)- group (wherein (c₀) is as defined above);
(11) the K₀ group: an A₁₀-N((O)ₙR₁)—CO—R₆— group, wherein A₁₀ represents a hydrogen atom (provided that n is not 0), an A₇″-SO₂— group (wherein A₇″ is as defined above), an A₈-SO₂— group (wherein A₈ is as defined above, provided that A₈ is not a hydrogen atom), an A₉′O— group (wherein A₉′ is as defined above, (provided that n is not 1), an A₉′- group (wherein A₉′ is as defined above, provided that A₈′ is excluded when n is 0), a R₂OCH₂— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above;
(12) the L₀ group:
an A₁₀′-N((O)ₙR₁)—SO₂—R₆— group [wherein A₁₀′ represents a hydrogen atom (provided that n is not 1), an A₉′O— group (wherein A₉′ is as defined above, provided that n is not 0), an A₉′— group (wherein A₉′ is as defined above, provided that A₈′ is excluded when n is 0), a R₂—CO— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above],
an A₀″R₁N—SO₂—N((O)ₙR₁′)—R₆— group [wherein A₉″ represents a hydrogen atom or an A₉′— group (wherein A₉′ is as defined above), and R₁, n, R₁′ and R₆ are as defined above] and
a (b₀)-SO₂—N((O)ₙR₁′)—R₆— group [wherein (b₀), n, R₁′ and R₆ are as defined above];
(13) the M₀ group:
a R₁(R₂S)C═N—R₆— group (wherein R₁, R₂ and R₆ are as defined above),
a R₂B(R₂′B′)C═N—R₆— group (wherein R₂ and R₆ are as defined above, R₂′ is the same as or different from R₂ and has the same meaning as R₂ has, and B and B′ are the same or different and represent an oxy group or a thio group),
a R₁R₁′N—(R₂S)C═N—R₆— group (wherein R₁, R₁′, R₂ and R₆ are as defined above),
a R₁N═C(SR₂)—NR₂′—R₆— group (wherein R₁, R₂, R₂′ and R₆ are as defined above), and
a R₁(R₁′O)N—R₆— group (wherein R₁, R₁′ and R₆ are as defined above);
(14) the N₀ group: a A₁₁-P(═O)(OR₁′)—R₄— group, wherein A₁₁ represents a R₁— group (wherein R₁ is as defined above), a R₁O—R₆— group (wherein R₁ and R₆ are as defined above) or a R₁OCO—CHR₀— group (wherein R₁ and R₀ are as defined above), and R₁′ and R₄ are as defined above;
III. in (Y_{A0})_{q}, Y_{A0} is a substituent on a carbon atom and represents a group included in the following X₀ group and Y₀ group, q represents 0, 1, 2, 3, 4 or 5, the sum of p (wherein p is as defined above) and q is not more than 5, and when q is not less than 2, Y_{A0}s are the same or different, and when q is not less than 2, adjacent two same or different Y_{A0}s may together form a group included in the Z₀ group to be fused to the A0 ring;
(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a Rₑ—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ— CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y₀ group: a M_{b0}-R_{d}— group, wherein M_{b0} represents a M_{c0}- group [wherein M_{c0} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by 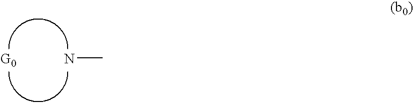
(wherein (b₀) is as defined above), a (c₀)- group represented by 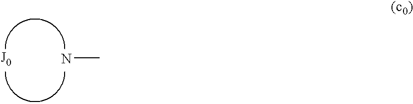
(wherein, (c₀) is as defined above), a (d₀)- group represented by 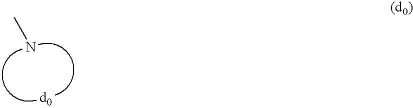
{wherein d₀ forms a 5 to 12-membered hydrocarbon ring which is substituted with a carbonyl group or a thiocarbonyl group and further which may be optionally substituted with an oxy group, a thio group, a —NR₁— group (wherein R₁ is as defined above), a sulfinyl group or a sulfonyl group}, or a (e₀)- group represented by 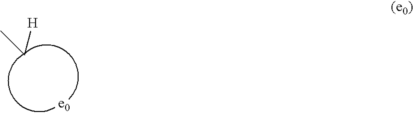
{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}, and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above), or a M_{c0}RₑN—SO₂— group (wherein M_{c0} and Rₑ are as defined above), and R_{d} is as defined above;
(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the A0 ring; and
IV. B0 is a group represented by formula (III-1): 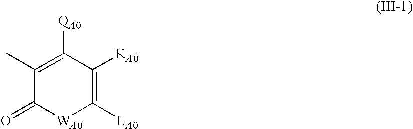
[wherein,
(1) Q_{A0} represents a hydroxyl group, a (b₀)- group (wherein (b₀) is as defined above), an A₉-B₆—B_{c}— group [wherein A₉ and B₆ are as defined above, and B_{c} represents an oxy group or a N—((O)ₘR₁′)— group (wherein m and R₁ are as defined above), provided that B_{c} is not a sulfonyl group when A₉ is a hydrogen atom], an A₇″-SO₂—B_{c}— group (wherein A₇″ and B_{c} are as defined above), an A₈-SO₂—B_{c}— group (wherein A₈ and B_{c} are as defined above, provided that A₈ is not a hydrogen atom), a R₁R₁′N—SO₂—B_{c}— group (wherein R₁, R₁′ and B_{c} are as defined above), a (b₀)-SO₂—B_{c}— group (wherein (b₀) and B_{c} are as defined above), an A₉′-B_{c}— group (wherein A₉′ and B_{c} are as defined above), a D₅-R₄—B_{c}— group (wherein D₅, R₄ and B_{c} are as defined above), a M_{c0}-B₃—B_{c}— group (wherein M_{c0}, B₃ and B_{c} are as defined above) or a M_{c0}-B_{c}— group (wherein M_{c0} and B_{c} are as defined above),
(2) W_{A0} represents an oxygen atom or a —NT_{A0}- group wherein T_{A0} represents a hydrogen atom, an A₉′- group (wherein A₉′ is as defined above), a D₅-R₄— group (wherein D₅ and R₄ are as defined above) or a M_{c0}- group (wherein M_{c0} is as defined above),
(3) K_{A0} and L_{A0} form a —V_{A0}═V_{A0}′—V_{A0}″═V_{A0}″— group {wherein V_{A0}, V_{A0}′, V_{A0}″, and V_{A0}′″ are the same or different, and represent a methine group optionally substituted with a Mₐ- group (wherein Mₐ is as defined above), or a —N═ group, and at least one of V_{A0}, V_{A0}′, V_{A0}″, and V_{A0}′″ represents a —N═ group},
a group represented by formula (III-2): 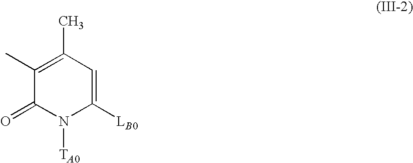
[wherein T_{A0} is as defined above, and L_{B0} represents a hydroxyl group or a methyl group],
a group represented by formula (III-3): 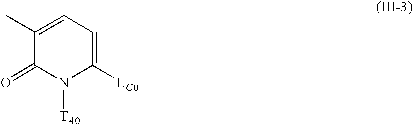
[wherein T_{A0} is as defined above, and L_{C0} represents a C1-C10 alkyl group],
a group represented by formula (III-4): 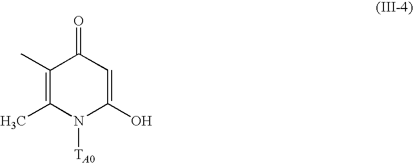
[wherein T_{A0} is as defined above],
a group represented by formula (III-5): 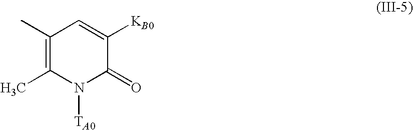
[wherein T_{A0} is as defined above, and K_{B0} represents a cyano group or a UOCO— group (wherein U represents a hydrogen atom or a C1-C10 alkyl group)],
a group represented by formula (III-6): 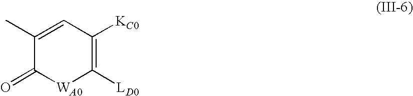
[wherein W_{A0} is as defined above, and K_{C0} and L_{D0} form a C3-C10 alkylene group, or a C4-C10 alkenylyne optionally substituted with 1 or more same or different Mₐ- groups (wherein Mₐ is as defined above)],
a group represented by formula (III-7): 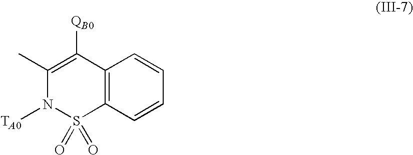
[wherein T_{A0} is as defined above, and Q_{B0} represents a hydroxyl group, a A₉-B₆—O— group (wherein A₉ and B₆ are as defined above), an A₇″-SO₂—O— group (wherein A₇″ is as defined above), an A₈-SO₂—O— group, (wherein A₈ is as defined above, provided that A₉ is not a hydrogen), a R₁R₁′N—SO₂—O— group (wherein R₁ and R₁′ are as defined above), a (b₀)-SO₂—O— group (wherein (b₀) is as defined above), an A₉′-O— group (wherein A₉′ is as defined above), a D₅-R₄—O— group (wherein D₅ and R₄ are as defined above), a M_{c0}-B₃—O— group (wherein M_{c0} and B₃ are as defined above) or a M_{c0}-O— group (wherein M_{c0} is as defined above)], or
a group represented by formula (III-8): 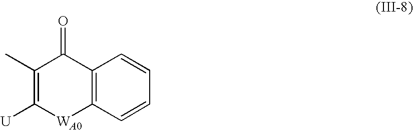
[wherein U and W_{A0} are as defined above]; and
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

4. A cinnamoyl compound represented by the formula (IV): 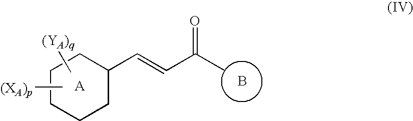
wherein,
I. A represents a benzene ring or a pyridine ring;
II. in (X_{A})ₚ, X_{A} is a substituent on a carbon atom, and represents a group included in any group of the following A group to N group, p represents 0, 1, 2, 3, 4 or 5 and, when p is not less than 2, X_{A}s are the same or different;
(1) the A group:
a D₁-R₄— group, wherein D₁ represents a (R₁—(O)ₖ-(A₁N—(O)ₖ′— group [wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), or a C3-C10 alkenyl group, or a C3-C10 alkynyl group, k represents 0 or 1, A₁ represents a R₃—(CHR₀)ₘ—(B₂—B₃)ₘ′— group {wherein R₃ represents a hydrogen atom, or a C1-C10 alkyl group optionally substituted with a halogen atom or a R₂—B₁— group (wherein R₂ and B₁ are as defined above), or a C2-C10 alkenyl group, or a C2-C10 alkynyl group, R₀ represents a hydrogen atom, a C1-C10 alkyl group or a C2-C10 haloalkyl group, m represents 0 or 1, B₂ represents a single bond, an oxy group, a thio group or a —N((O)ₙR₁′)— group (wherein R₁′ is the same as or different from R₁ and has the same meaning as R₁ has, and n represents 0 or 1), B₃ represents a carbonyl group, a thiocarbonyl group or a sulfonyl group, m′ represents 0 or 1, and B₃ is not a sulfonyl group when m is 0 and R₃ is a hydrogen atom}, and k′ represents 0 or 1], and R₄ represents a C1-C10 alkylene group, provided that a R₀′R₀″N—R₄— group (wherein R₀′ and R₀″ are the same as or different from R₀ and have the same meaning as R₀ has, and R₄ is as defined above) is excluded,
a D₂-R₄— group, wherein D₂ represents a cyano group, a R₁R₁′NC(═N—(O)ₙ-A₁)- group (wherein R₁, R₁′, n and A₁ are as defined above), an A₁N═C(—OR₂)— group (wherein A₁ and R₂ are as defined above) or a NH₂—CS— group, and R₄ is as defined above,
a D₃-R₄— group, wherein D₃ represents a nitro group or a R₁OSO₂— group (wherein R₁ is as defined above), and R₄ is as defined above, and
a R₀SO₂— group, wherein R₁ is as defined above;
(2) the B group: an (a)-group represented by 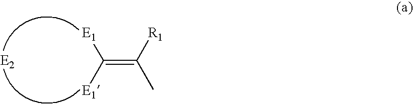
wherein E₁ and E₁′ represent a methylene group optionally substituted with a C1-C10 alkyl group or a C1-C10 alkoxy group, or a carbonyl group, provided that E₁ and E₁′ are not a carbonyl group at the same time, E₂ represents a C2-C10 alkylene group optionally substituted with an oxy group, a thio group, a sulfinyl group, a sulfonyl group or a —NR₁′— group (wherein R₁′ is as defined above), or a C3-C10 alkenylene group optionally substituted with an oxy group, a thio group, a sulfinyl group, a sulfonyl group or a —NR₁′— group (wherein R₁′ is as defined above), and R₁ is as defined above;
(3) the C group: a C2-C10 alkenyl group substituted with a halogen atom, a R₂—B₁— group (wherein R₂ and B₁ are as defined above), a D₄-R₄— group [wherein D₄ represents a hydroxyl group or an A₁-O— group (wherein A₁ is as defined above), and R₄ is as defined above], a D₅- group [wherein D₅ represents an O═C(R₃)— group (wherein R₃ is as defined above), an A₁-(O)ₙ—N═C(R₃)— group (wherein A₁, n and R₃ are as defined above), a R₁—B₀—CO—R₄—(O)ₙ—N═C(R₃)— group {wherein R₁, R₄, n and R₃ are as defined above, and B₀ represents an oxy group, a thio group or a —N((O)ₘR₁′)— group (wherein R₁′ and m are as defined above)}, a D₂-R₄—(O)ₙ—N═C(R₃)— group (wherein D₂, R₄, n and R₃ are as defined above) or a R₁A₁N—N═C(R₃)— group (wherein R₁, A₁ and R₃ are as defined above)], a R₁A₁N—O—R₄— group (wherein R₁, A₁ and R₄ are as defined above), a R₁ (A₁-(O)ₙ—)N— group (wherein R₁, A₁ and n are as defined above), a D₂- group (wherein D₂ is as defined above) or a D₃- group (wherein D₃ is as defined above);
(4) the D group: a C2-C10 alkynyl group substituted with a (b)-R₄— group
[wherein in (b) 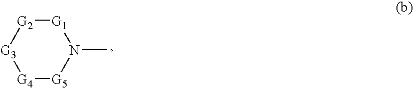
G₁, G₂, G₄ and G₅ represent a methylene group which is connected with the adjacent atom via a single bond and which may be optionally substituted with a methyl group, or a methine group which is connected with the adjacent atom via a double bond and which may be optionally substituted with a methyl group, and G₃ represents a single bond, or a double bond, or a C1-C10 alkylene group optionally substituted with a methyl group, an oxy group, a thio group, a sulfinyl group, a sulfonyl group or a —NR₁— group (wherein R₁ is as defined above), or a C2-C10 alkenylene group optionally substituted with a methyl group, an oxy group, a thio group, a sulfinyl group, a sulfonyl group or a —NR₁— group (wherein R₁ is as defined above); and R₄ is as defined above], a (c) —R₄— group [wherein, in (c) 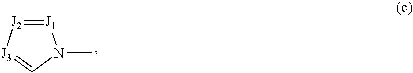
J₁, J₂ and J₃ are the same or different, and represent a methine group optionally substituted with a methyl group, or a nitrogen atom; and R₄ is as defined above), a halogen atom, a R₂—B₁—R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above) or a D₃-R₄— group (wherein D₃ and R₄ are as defined above);
(5) the E group: an A₂-CO—R₅— group, provided that R₅ is not a vinylene group when A₂ is a hydroxyl group,
[wherein A₂ represents
(i) an A₃-B₄— group
wherein A₃ represents a hydrogen atom, or a C1-C10 alkyl group, or a C2-C10 haloalkyl group, or a C2-C10 alkenyl group optionally substituted with a halogen atom, or a C3-C10 alkynyl group optionally substituted with a halogen atom, or Rₐ—(R₄)ₘ— group (wherein Rₐ represents a phenyl group, a pyridyl group, a furyl group or a thienyl group, which may be optionally substituted with a halogen atom, a C1-C10 alkyl group, a C1-C10 alkoxy group or a nitro group, and R₄ and m are as defined above), or a C1-C10 alkyl group substituted with a (b) —R₄— group (wherein (b) and R₄ are as defined above), a (c) —R₄— group (wherein (c) and R₄ are as defined above), a R₂—B —R₄— group (wherein R₂, B₁ and R₄ are as defined above), a D₄-R₄— group (wherein D₄ and R₄ are as defined above), a D₅- group (wherein D₅ is as defined above), a D₁-R₄— group (wherein D₁ and R₄ are as defined above), a D₂- group (wherein D₂ is as defined above), a D₃-R₄— group (wherein D₃ and R₄ are as defined above) or an A₄-SO₂—R₄— group {wherein A₄ represents a (b)- group (wherein (b) is as defined above), a (c)- group (wherein (c) is as defined above) or a R₁R₁′N— group (wherein R₁ and R₁′ are as defined above), and R₄ is as defined above}, and B₄ represents an oxy group, a thio group or a —N((O)ₘR₁′)— group (wherein R₁ and m are as defined above), provided that A₃ is not a hydrogen atom when B₄ is a thio group;
(ii) a R₁—B₄—CO—R₄—B₄′— group, wherein R₁, B₄ and R₄ are as defined above, B₄′ is the same as or different from B₄ and has the same meaning as B₄ has, provided that R₂ is not a hydrogen atom when B₄ is a thio group, or
a D₂-R₄—B₄— group, wherein D₂, R₄ and B₄ are as defined above;
(iii) a R₂—SO₂—NR₁— group, wherein R₂ is as defined above, provided that a hydrogen atom is excluded, and R₁ is as defined above;
(iv) a (b)- group, wherein (b) is as defined above,
(v) a (c)- group, wherein (c) is as defined above, or
(vi) a R₁A₁N—NR₁′ group, wherein R₁, A₁ and R₁′ are as defined above; and
R₅ represents a C2-C10 alkenylene group optionally substituted with a halogen atom or a C2-C10 alkynylene group];
(6) the F group: an A₅-B₅—R₆— group
[wherein A₅ represents a C2-C10 alkyl group substituted with a D₄- group (wherein D₄ is as defined above), a D₁- group (wherein D₁ is as defined above), a D₃- group (wherein D₃ is as defined above) or an A₄-SO₂— group (wherein A₄ is as defined above), or a C1-C10 alkyl group substituted with a R₂—B₁— group (wherein R₂ and B₁ are as defined above), a D₂- group (wherein D₂ is as defined above), a D₅- group (wherein D₅ is as defined above) or an A₂-CO— group (wherein A₂ is as defined above),
B₅ represents a B₁— group (wherein B₁ is as defined above) or a —NA₁- group (wherein A₁ is as defined above), and
R₆ represents a single bond or a C1-C10 alkylene group];
(7) the G group: an A₆-B₅—R₆— group
[wherein A₆ represents an (a) —R₄— group (wherein (a) and R₄ are as defined above), or a C2-C10 alkenyl group, or a C2-C10 alkynyl group, or a C2-C10 alkenyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as define above), or a C2-C10 alkynyl group substituted with a halogen atom, a R₂—B₁— group (wherein R₂ and B₁ are as defined above), a D₅- group (wherein D₅ is as defined above), a D₂- group (wherein D₂ is as defined above) or an A₂-CO— group (wherein A₂ is as defined above), or a C3-C10 alkenyl group substituted with a (b)- group (wherein (b) is as defined above), a (c)- group (wherein (c) is as defined above), a D₄- group (wherein D₄ is as defined above), a D₁-group (wherein D₁ is as defined above) or a D₃- group (wherein D₃ is as defined above), or a C3-C10 alkynyl group substituted with a D₄- group (wherein D₄ is as defined above), a D₁- group (wherein D₁ is as defined above) or a D₃- group (wherein D₃ is as defined above), and B₅ and R₆ are as defined above];
(8) the H group:
a D₂-N(—(O)ₙ-A₁)-R₆— group, wherein D₂, n, A₁ and R₆ are as defined above,
a D₂- group, wherein D₂ is as defined above, provided that a cyano group is excluded,
a R₁(R₁′(O)ₙ)N—CR₁″═N—R₆— group, wherein R₁, R₁′, n and R₆ are as defined above, R₁ ″ is the same as or different from R₁ and has the same meaning as R₁ has,
a R₁—(O)ₙ—N═CR₁′—NR₂—R₆— group, wherein R₁, n, R₁′, R₂ and R₆ are as defined above,
a R₂—B₃—NR₁—CO—NR₁′—R₆— group, wherein R₂, B₃, R₁, R₁′ and R₆ are as defined above,
a D₂-CO—NR₁—R₆— group, wherein D₂, R₁ and R₆ are as defined above, or
an A₂-COCO—NR₁—R₆— group, wherein A₂, R₁ and R₆ are as defined above;
(9) the I group:
an A₇-B₆—N((O)ₙR₁)—R₆— group [wherein A₇ represents a C2-C10 alkenyl group optionally substituted with a halogen atom, or a C2-C10 alkynyl group, or a C3-C10 haloalkynyl group, or a R₂—B —R₄— group (wherein R₂, B₁ and R₄ are as defined above), or a D₄-R₄— group (wherein D₄ and R₄ are as defined above), or a D₅-R₄— group (wherein D₅ and R₄ are as defined above), or a D₁-R₄— group (wherein D₁ and R₄ are as defined above), or a (b) —R₄— group (wherein (b) and R₄ are as defined above), or a (c) —R₄— group (wherein (c) and R₄ are as defined above), or a D₂-R₄— group (wherein D₂ and R₄ are as defined above), or a D₃-R₄— group (wherein D₃ and R₄ are as defined above), or an A₄-SO₂—R₄— group (wherein A₄ and R₄ are as defined above), or an A₂-CO—R₄— group (wherein A₂ and R₄ are as defined above), B₆ represents a carbonyl group or a thiocarbonyl group, and n, R₁ and R₆ are as defined above],
an A₈-CS—N((O)ₙR₁′)—R₆— group [wherein A₈ represents a hydrogen atom or a C1-C10 alkyl group optionally substituted with a halogen atom, and n, R₁ and R₆ are as defined above],
an A₇′-B₂′—B₃—N((O)ₙR₁′)—R₆— group [wherein A₇′ represents a C3-C10 alkenyl group optionally substituted with a halogen atom, or a C3-C10 alkynyl group optionally substituted with a halogen atom, or a R₂—B₁—R₄′— group (wherein R₂ and B₁ are as defined above, and R₄′ represents a C2-C10 alkylene group), or a D₄-R₄′— group (wherein D₄ and R₄′ are as defined above), or a D₁-R₄′— group (wherein D₁ and R₄′ are as defined above), or a (b) —R₄′— group (wherein (b) and R₄′ are as defined above), or a (c) —R₄′— group (wherein (c) and R₄′ are as defined above), or a D₂-R₄— group (wherein D₂ and R₄ are as defined above), or a D₃-R₄′— group (wherein D₃ and R₄′ are as defined above), or an A₂-CO—R₄— group (wherein A₂ and R₄ are as defined above), B₂′ represents an oxy group, a thio group or a —N((O)ₙ.R₁′)— group (wherein n′ is the same as or different from n and has the same meaning as n has, and R₁′ is as defined above), and B₃, n, R₁ and R₆ are as defined above],
an A₈′-B₂′-CS—N((O)ₙR₁′)—R₄— group [wherein A₈′ represents a C1-C10 alkyl group or a C2-C10 haloalkyl group, B₂′ is as defined above, and n, R₁ and R₆ are as defined above],
an A₈′-S—B₃′—N((O)ₙR₁′)—R₆— group [wherein A₈′, n, R₁ and R₆ are as defined above, and B₃′ represents a carbonyl group or a sulfonyl group] or an A₇″-SO₂—N((O)ₙR₁′)—R₆— group [wherein A₇″ represents a C2-C10 alkenyl group, or a C3-C10 alkenyl group substituted with a halogen atom, or a C3-C10 alkynyl group optionally substituted with a halogen atom, or a R₂—B₁—R₄′— group (wherein R₂, B₁ and R₄′ are as defined above), or a D₄-R₄′— group (wherein D₄ and R₄′ are as defined above), or a D₅-R₄— group (wherein D₅ and R₄ are as defined above), or a D-R₄′— group (wherein D₁ and R₄′ are as defined above), or a (b) —R₄′— group (wherein (b) and R₄′ are as defined above), or a (c) —R₄′— group (wherein (c) and R₄′ are as defined above), or a D₂-R₄— group (wherein D₂ and R₄ are as defined above), or a NO₂—R₄— group (wherein R₄ is as defined above), or an A₂-CO—R₄— group (wherein A₂ and R₄ are as defined above), and n, R₁ and R₄ are as defined above];
(10) the J group:
an A₇-CO— group (wherein A₇ is as defined above),
an A₉-CS— group (wherein A₉ represents A₇ or A₈),
an A₉′(O)ₘN═C(A₉)- group (wherein A₉′ represents A₇′ or A₈′, and m and A₉ are as defined above),
a D₂-CO— group (wherein D₂ is as defined above),
an A₂-COCO— group (wherein A₂ is as defined above),
an A₉-CO—B₁′—R₆— group (wherein A₉ and R₆ are as defined above, and B₁′ represents an oxy group or a thio group, provided that A₉ is not A₈ when B₁′ is an oxy group),
an A₉-CS—B₁′—R₆— group (wherein A₉, B₁′ and R₆ are as defined above),
an A₇″-SO₂—B₁′—R₆— group (wherein A₇″, B₁′ and R₆ are as defined above),
an A₈-SO₂—B₁′—R₆— group (wherein A₈, B₁′ and R₆ are as defined above, provided that A₈ is not a hydrogen atom),
an A₉′-B₂′—B₃—B₁′—R₆— group (wherein A₉′, B₂′, B₃, B₁′ and R₆ are as defined above), and
a C2-C10 alkenyl group substituted with a (b)- group (wherein (b) is as defined above) or a (c)- group (wherein (c) is as defined above);
(11) the K group: an A₁₀-N((O)ₙR₁)—CO—R₆— group
[wherein A₁₀ represents a hydrogen atom (provided that n is not 0), an A₇″-SO₂— group (wherein A₇″ is as defined above), an A₈-SO₂— group (wherein A₈ is as defined above, provided that A₈ is not a hydrogen atom), an A₉′O— group (wherein A₉′ is as defined above, provided that n is not 1), an A₉′- group (wherein A₉′ is as defined above, provided that A₈′ is excluded when n is 0), a R₂OCH₂— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above];
(12) the L group:
an A₁₀′-N((O)ₙR₁)—SO₂—R₆— group [wherein A₁₀′ represents a hydrogen atom (provided that n is not 0), an A₉′O— group (wherein A₉′ is as defined above, provided that n is not 1), an A₉′- group (wherein A₉′ is as defined above, provided that A₈′ is excluded when n is 0), a R₂—CO— group (wherein R₂ is as defined above), an A₂-CO—R₄— group (wherein A₂ and R₄ are as defined above) or an A₂-CO—CH(CH₂CO-A₂)- group (wherein A₂ is as defined above), and n, R₁ and R₆ are as defined above],
an A₉″R₁N—SO₂—N((O)ₙR₁′)—R₆— group [wherein A₉″ represents a hydrogen atom or an A₉′- group (wherein A₉′ is as defined above), and R₁, n, R₁′ and R₆ are as defined above], and
a (b)-SO₂—N((O)ₙR₁′)—R₆— group [wherein (b), n, R₁′ and R₆ are as defined above];
(13) the M group:
a R₁(R₂S)C═N—R₆— group (wherein R₁, R₂ and R₆ are as defined above),
a R₂B(R₂′B′)C═N—R₆— group (wherein R₂ and R₆ are as defined above, R₂′ is the same as or different from R₂ and has the same meaning as R₂ has, and B and B′ are the same or different and represent an oxy group or a thio group),
a R₁R₁′N—(R₂S)C═N—R₆— group (wherein R₁, R₁′, R₂ and R₆ are as defined above),
a R₁N═C(SR₂)—NR₂′—R₆— group (wherein R₁, R₂, R₂′ and R₆ are as defined above), and
a R₁(R₁′O)N—R₆— group (wherein R₁, R₁′ and R₆ are as defined above);
(14) the N group: an A₁₁-P(═O)(OR₁′)—R₄— group
[wherein A₁₁ represents a R₁— group (wherein R₁ is as defined above), a R₁O—R₆— group (wherein R₁ and R₆ are as defined above) or a R₁OCO—CHR₀— group (wherein R₁ and R₀ are as defined above), and R₁′ and R₄ are as defined above];
III. in (Y_{A})_{q}, Y_{A} is a substituent on a carbon atom and represents a group included in the following X group or Y group, q represents 0, 1, 2, 3, 4 or 5, and the sum of p (wherein p is as defined above) and q is not more than 5, and when q is not less than 2, Y_{A}s are the same or different, and when q is not less than 2, adjacent two same or different Y_{A}s may together form a group included in the Z group to be fused to the A ring;
(1) the X group: a Mₐ-group, wherein Mₐ represents a R_{b}— group (wherein R_{b} represents a C1-C10 alkyl group optionally substituted with a halogen atom), a halogen atom, a nitro group, a cyano group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}— SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y group: a M_{b}-R_{d}— group, wherein M_{b} represents a M_{c}-group [wherein M_{c} represents a M_{d}-R_{d}′— group {wherein M_{d} represents a phenyl group optionally substituted with a Mₐ- group (wherein Mₐ is as defined above), a pyridyl group optionally substituted with a Mₐ- group (wherein Mₐ is as defined above), a naphthyl group optionally substituted with a Mₐ- group (wherein Mₐ is as defined above), a (b)- group (wherein (b) is as defined above), a (c)- group (wherein (c) is as defined above), a (d)- group 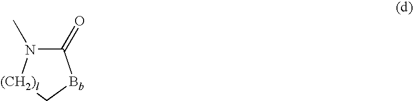
(wherein l is 2, 3 or 4, B_{b} represents an oxy group or a thio group) or an (e)- group 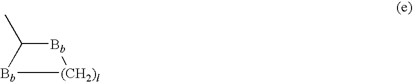
(wherein l and B_{b} are as defined above)}, and R_{d}′ is the same as or different from R_{d} and has the same meaning as R_{d} has], a M_{c}-Bₐ— group (wherein M_{c} and Bₐ are as defined above), a M_{c}-CO— group (wherein M_{c} is as defined above), a M_{c}-CO—O— group (wherein M_{c} is as defined above), a M_{c}O—CO— group (wherein M_{c} is as defined above), a M_{c}RₑN— group (wherein M_{c} and Rₑ are as defined above), a M_{c}-CO—NRₑ— group (wherein M_{c} and Rₑ are as defined above), a M_{c}O—CO—NRₑ— group (wherein M_{c} and Rₑ are as defined above), a M_{c}RₑN—CO— group (wherein M_{c} and Rₑ are as defined above), a M_{c}RₑN—CO—NRₑ′— group (wherein M_{c}, Rₑ and Rₑ′ are as defined above), a M_{c}RₑN—C(═NRₑ′)—NRₑ″— group (wherein M_{c}, Rₑ, Rₑ′ and Rₑ″ are as defined above), a M_{c}-SO₂—NRₑ— group (wherein M_{c} and Rₑ are as defined above) or a M_{c}RₑN—SO₂— group (wherein M_{c} and Rₑ are as defined above), and
R_{d} is as defined above;
(3) the Z group: a —N═C(Yₐ)—Yₐ′— group (wherein Yₐ represents a hydrogen atom, or a C1-C10 alkyl group optionally substituted with a halogen atom, or a C1-C10 alkoxy group, and Yₐ′ represents an oxy group, or a thio group, or an imino group optionally substituted with a C1-C10 alkyl group),
a —Y_{b}—Y_{b}′—Y_{b}″— group (wherein Y_{b} and Y_{b}″ are the same or different, and represent a methylene group, or an oxy group, or a thio group, or a sulfinyl group, or an imino group optionally substituted with a C1-C10 alkyl group, and Y_{b}′ represents a C1-C4 alkylene group optionally substituted with a halogen atom, or a C1-C4 alkylene group optionally substituted with an oxo group), or
a —Y_{c}—O—Y_{c}′—O— group (wherein Y_{c} and Y_{c}′ are the same or different, and represent a C1-C10 alkylene group);
IV. B is
a group represented by formula (IV-1): 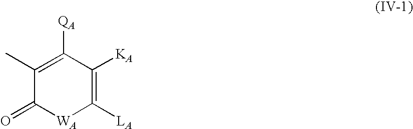
wherein,
(1) Q_{A} represents a hydroxyl group, a (b)- group (wherein (b) is as defined above), an A₉-B₆—B_{c}— group [wherein A₉ and B₆ are as defined above, and B_{c} represents an oxy group or a —N((O)ₘR₁′)— group (wherein m and R₁ are as defined above), provided that B_{c} is not a sulfonyl group when A₉ is a hydrogen atom], an A₇″-SO₂—B_{c}— group (wherein A₇″ and B_{c} are as defined above), an A₈-SO₂—B_{c}— group (wherein A₈ and B_{c} are as defined above, provided that A_{B} is not a hydrogen atom), a R₁R₁′N—SO₂—B_{c}— group (wherein R₁, R₁′ and B_{c} are as defined above), a (b₀)-SO₂—O— group (wherein (b) and B_{c} are as defined above), an A₉′-B_{c}— group (wherein A₉′ and B_{c} are as defined above), a D₅-R₄—B_{c}— group (wherein D₅, R₄ and B_{c} are as defined above), a M_{c}-B₃—B_{c}— group (wherein M_{c}, B₃ and B_{c} are as defined above) or a M_{c}-B_{c}— group (wherein M_{c} and B_{c} are as defined above),
(2) W_{A} represents an oxygen atom or a —NT_{A}- group [wherein T_{A} represents a hydrogen atom, an A₉′- group (wherein A₉′ is as defined above), a D₅-R₄— group (wherein D₅ and R₄ are as defined above) or a M_{c}- group (wherein M_{c} is as defined above)], and
(3) K_{A} and L_{A} form a —V_{A}═V_{A}′—V_{A}″═V_{A}′″— group {wherein V_{A}, V_{A}′, V_{A}″ and V_{A}′″ are the same or different, and represent a methine group optionally substituted with a Mₐ-group (wherein Mₐ is as defined above), or a —N═ group, and at least one of V_{A}, V_{A}′, V_{A}″ and V_{A}′″ represents a —N═ group};
a group represented by formula (IV-2): 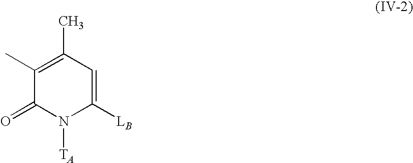
wherein T_{A} is as defined above, and L_{B} represents a hydroxyl group or a methyl group;
a group represented by formula (IV-3): 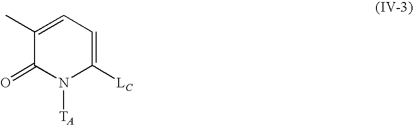
wherein T_{A} is as defined above, and L_{C} represents a C1-C10 alkyl group;
a group represented by formula (IV-4): 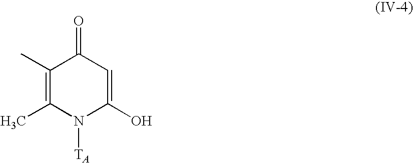
wherein T_{A} is as defined above;
a group represented by formula (IV-5): 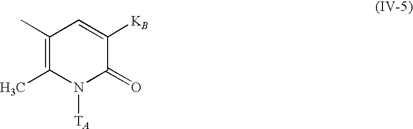
wherein T_{A} is as defined above, and K_{B} represents a cyano group or a UOCO— group (wherein U represents a hydrogen atom or C1-C10 alkyl group);
a group represented by formula (IV-6): 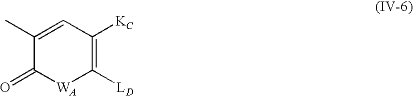
wherein W_{A} is as defined above, and K_{C} and L_{D} form a C3-C10 alkylene group, or a C4-C10 alkenylene group optionally substituted with 1 or more same or different Mₐ- groups (wherein Mₐ is as defined above);
a group represented by formula (IV-7): 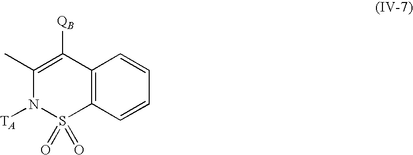
wherein T_{A} is as defined above, Q_{B} represents a hydroxyl group, an A₉-B₆—O— group [wherein A₉ and B₆ are as defined above], an A₇″-SO₂—O— group (wherein A₇″ is as defined above), an A₈-SO₂—O— group (wherein A₈ is as defined above, provided that A₈ is not a hydrogen atom), a R₁R₁′N—SO₂—O— group (wherein R₁ and R₁′ are as defined above), a (b)-SO₂—O— group (wherein (b) is as defined above), an A₉′-O— group (wherein A₉′ is as defined above), a D₅-R₄—O— group (wherein D₅ and R₄ are as defined above), a M_{c}-B₃—O— group (wherein M_{c} and B₃ are as defined above), or a M_{c}-O— group (wherein M_{c} is as defined above); or
a group represented by formula (IV-8) 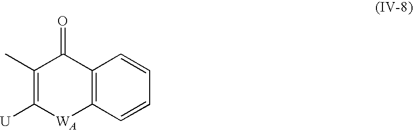
wherein U and W_{A} are as defined above; and
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range].

5. A cinnamoyl compound represented by the formula (V): 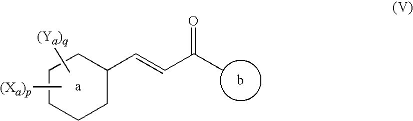
wherein,
I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a₁ represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)₁—CONH—CH₂— group (wherein r₀ represents a C1-C10 alkyl group, and 1 represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), or a C1-C10 alkyl group substituted with a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,
Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring;
II. b is
a group represented by formula (V-1): 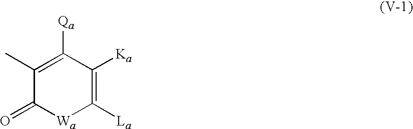
wherein Qₐ represents a rₐ-O— group {rₐ represents a hydrogen atom, or a C1-C10 alkyl group, or a C3-C10 alkenyl group, or a C3-C10 alkynyl, or a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r₁ is as defined above)}, or a piperidino group, or a morpholino group, or a r₄r₄′N-group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein Tₐ represents a r_{b}- group (wherein r_{b} is the same as or different from rₐ, and represents the same meaning as rₐ has) or a r₃′— group (wherein r₃′ is the same as or different from r₃, and represents the same meaning as r₃ has)], and Kₐ and Lₐ form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′, Vₐ″ and Vₐ′″ represents a —N═ group);
a group represented by formula (V-2): 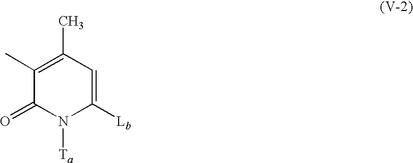
wherein Tₐ is as defined above, and L_{b} represents a hydroxyl group or a methyl group;
a group represented by formula (V-3): 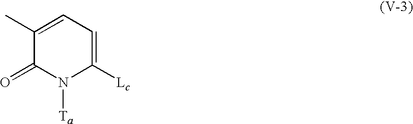
wherein Tₐ is as defined above, and L_{c} represents a C1-C10 alkyl group;
a group represented by formula (V-4): 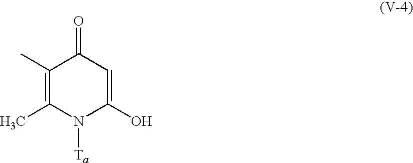
wherein Tₐ is as defined above;
a group represented by formula (V-5): 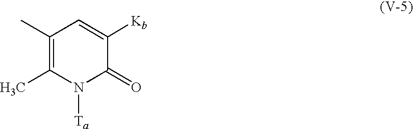
wherein Tₐ is as defined above, and K_{b} represents a cyano group or a UOCO— group (wherein U represents a hydrogen atom or C1-C10 alkyl group);
a group represented by formula (V-6): 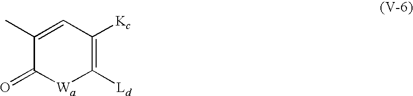
wherein Wₐ is as defined above, and K_{c} and L_{d} form a C3-C10 alkylene group or a C4-C10 alkenylene group;
a group represented by formula (V-7): 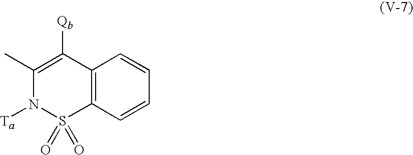
wherein Tₐ is as defined above, and Q_{b} represents a rₐ-O— group (wherein rₐ is as defined above); or
a group represented by formula (V-8): 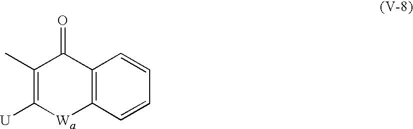
wherein U and Wₐ are as defined above; and
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

6. A process for producing a cinnamoyl compound represented by formula (VI′): 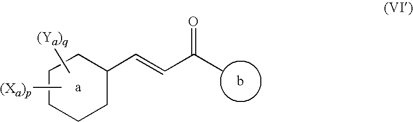
wherein a, Xₐ, Yₐ, p and q are defined below, and
b represents a group represented by formula (VI′-1): 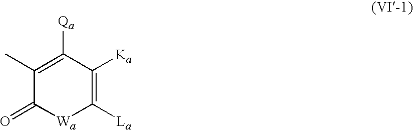
wherein Qₐ, Wₐ, Kₐ and Lₐ are as defined below, a group represented by formula (VI′-2): 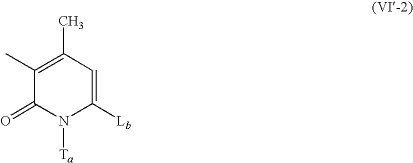
wherein Tₐ and L_{b} are as defined below, a group represented by formula (VI′-3): 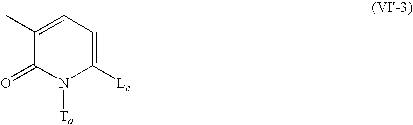
wherein Tₐ and L_{C} are as defined below, a group represented by formula (VI′-4): 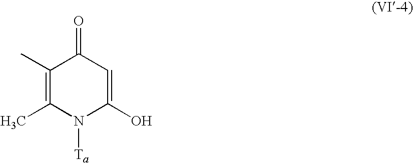
wherein Tₐ is as defined below, a group represented by formula (VI′-5): 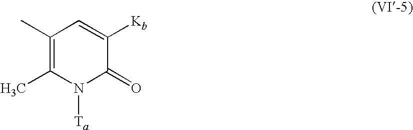
wherein Tₐ and K_{b} are as defined below, a group represented by formula (VI′-6): 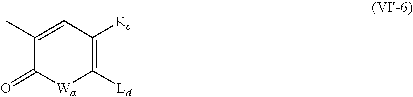
wherein Wₐ, K_{c} and L_{d} are as defined below, a group represented by formula (VI′-7): 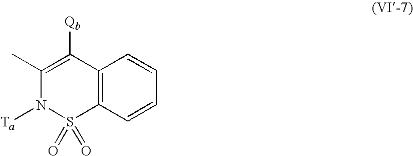
wherein Tₐ and Q_{b} are as defined below, or a group represented by formula (VI′-8): 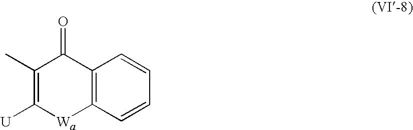
wherein U and Wₐ are as defined below; which comprises reacting a benzaldehyde derivative represented by the formula (VI): 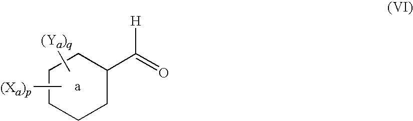
[wherein,
I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)₁—CONH—CH₂— group (wherein r₀ represents a C1-C10 alkyl group, and 1 represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), or a C1-C10 alkyl group substituted with a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,
Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring] with a compound represented by formula (VI-1): 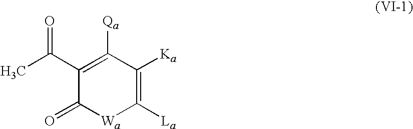
[wherein
Qₐ represents a rₐ-O— group {rₐ represents a hydrogen atom, or a C1-C10 alkyl group, or a C3-C10 alkenyl group, or a C3-C10 alkynyl, or a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r₁ is as defined above)}, or a piperidino group, or a morpholino group, or a r₄r₄′N— group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein Tₐ represents a r_{b}- group (wherein r_{b} is the same as or different from rₐ, and represents the same meaning as rₐ has) or a r₃′— group (wherein r₃′ is the same as or different from r₃, and represents the same meaning as r₃ has)], and Kₐ and L_{α} form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′Vₐ″ and Vₐ′″ represents a —N═ group)], a compound represented by formula (VI-2): 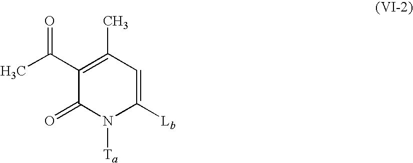
[wherein Tₐ is as defined above, and L_{b} represents a hydroxyl group or a methyl group], a compound represented by formula (VI-3): 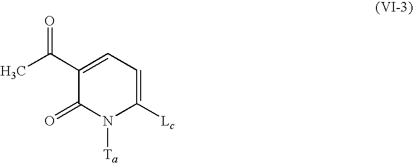
[wherein Tₐ is as defined above, and L_{c} represents a C1-C10 alkyl group], a compound represented by formula (VI-4): 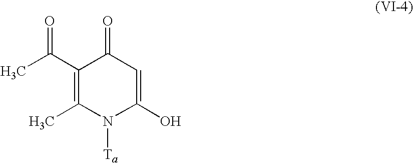
[wherein Tₐ is as defined above], a compound represented by formula (VI-5): 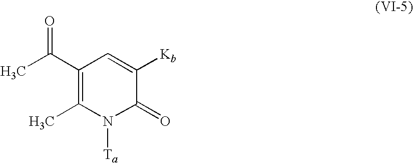
[wherein Tₐ is as defined above, and K_{b} represents a cyano group or a UOCO— group (wherein U represents a hydrogen atom or C1-C10 alkyl group)], a compound represented by formula (VI-6): 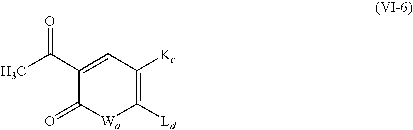
[wherein Wₐ is as defined above, and K_{c} and L_{d} form a C3-C10 alkylene group or a C4-C10 alkenylene group], a compound represented by formula (VI-7): 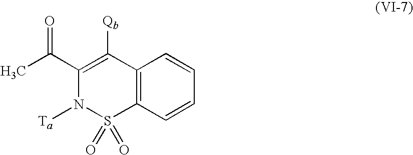
[wherein Tₐ is as defined above, and Q_{b} represents a rₐ-O— group (wherein rₐ is as defined above)], or a compound represented by formula (VI-8): 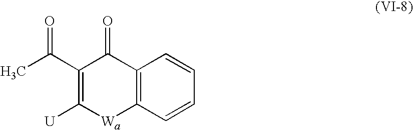
[wherein U and Wₐ are as defined above]; and
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

7. A process for producing a compound represented by formula (VII-3): 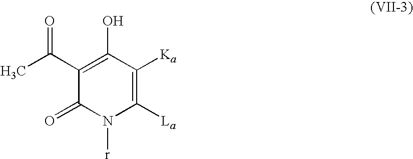
[wherein r, Kₐ and Lₐ are as defined below] which comprises 3-oxobutyrylamidating a compound represented by formula (VII-1): 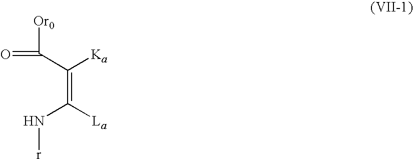
[wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group)] to produce a compound represented by formula (VII-2): 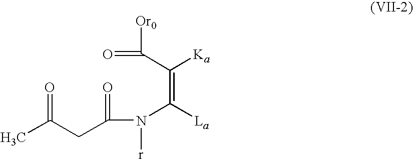
[wherein r, r₀, Kₐ and Lₐ are as defined above], and then cyclizing the compound represented by the formula (VII-2).

8. A process for producing a compound represented by formula (VIII-2): 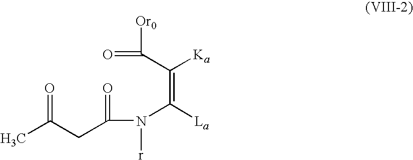
[wherein r, r₀, Kₐ and Lₐ are as defined below] which comprises 3-oxobutyrylamidating a compound represented by formula (VIII-1): 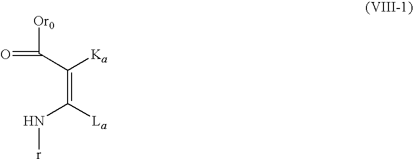
[wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group)].

9. A compound represented by the formula (IX): 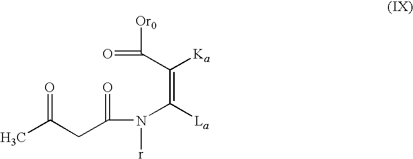
wherein r represents a hydrogen atom or a C1-C10 alkyl group, r₀ represents a C1-10 alkyl group, Kₐ and Lₐ form a —Vₐ═Vₐ′—Vₐ″═Vₐ′″— group (wherein Vₐ, Vₐ′, Vₐ″ and Vₐ′″ are the same or different, represent a methine group or a —N═ group, and at least one of Vₐ, Vₐ′, Vₐ″ and Vₐ′″ represents a —N═ group).

10. A compound represented by the formula (X): 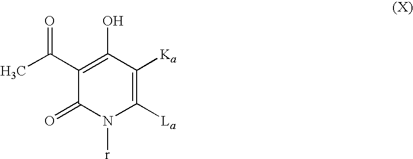
wherein r represents a hydrogen atom or a C1-C10 alkyl group, Kₐ and Lₐ form a —Vₐ═Vₐ′—Vₐ″═Vₐ′″— group (wherein Vₐ, Vₐ′, Vₐ″ and Vₐ′″ are the same or different and represent a methine group or a —N═ group, and at least one of Vₐ, Vₐ′, Vₐ″ and Vₐ′″ represents a —N═ group).

11. A process for producing a compound represented by formula (XI-2): 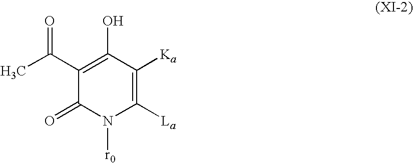
[wherein r₀ represents a C1-C10 alkyl group, and Kₐ and Lₐ are as defined below] which comprises alkylating a compound represented by formula (XI-1): 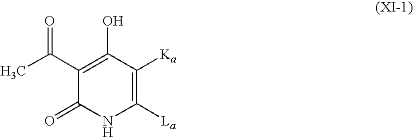
[wherein Kₐ and Lₐ form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′, Vₐ″ and Vₐ′″ represents a —N═ group)].

12. A composition for suppressing transcription of an extracellular matrix gene, which comprises an inert carrier and a cinnamoyl compound represented by formula (I′): 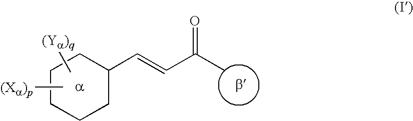
wherein,
I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})ₚ, X_{α} is a substituent on a carbon atom and represents a group which does not belong to the following X₀ group, Y₀ group and Z₀ group, p represents 0, 1, 2, 3, 4 or 5, and when p is not less than 2, X_{α}s are the same or different and the sum of p and q is not more than 5;
(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{d}—Bₐ—R_{d}— group (wherein R_{d} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a R_{d}—CO—R_{d}— group (wherein R_{d} represents a hydrogen atom, or a C1-C10 alkyl group optionally substituted with a halogen atom, and R_{d} is as defined above), a R_{d}— CO—O—R_{d}— group (wherein Rₑ and R_{d} are as defined above), a R_{d}O—CO—R_{d}— group (wherein Rₑ and R_{d} are as defined above), an HO—CO—CH═CH— group, a RₑRₑ′N—R_{d}— group (wherein Rₑ and R_{d}′ are the same or different, R_{d} is as defined above, R_{d}′ has the same meaning as R_{d} has, and R_{d} is as defined above), a R_{d}—CO—NR_{d}′—R_{d}— group (wherein Rₑ, Rₑ′ and R_{d} are as defined above), a R_{b}O—CO—N(R_{d})—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NR_{d}—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y₀ group: a M_{b0}-R_{d}— group, wherein M_{b0} represents a M_{c0}- group
[wherein M_{co} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by 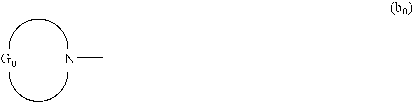
(wherein G₀ forms an optionally substituted, saturated or unsaturated, nonaromatic 5 to 14-membered hydrocarbon ring or heterocyclic ring), a (c₀)- group represented by 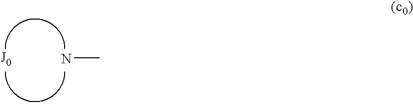
(wherein, J₀ forms a 5 to 7-membered aromatic ring optionally containing a nitrogen atom), a (d₀)- group represented by 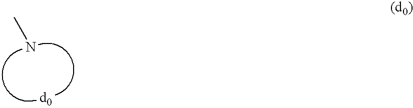
[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C10 alkenyl group or a C3-C10 alkynyl group, and B₁ represents an oxy group, a thio group, a sulfinyl group or a sulfonyl group), a C3-C10 alkenyl group, or a C3-C10 alkynyl group}, a sulfinyl group, or a sulfonyl group], or a (e₀)- group represented by 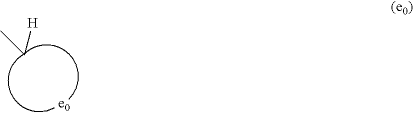
{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}; and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}-CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;
(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and
II. β′ represents
a group represented by formula (I′-1): 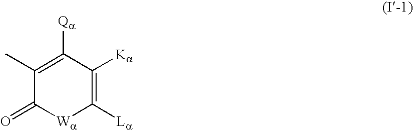
wherein,
(1) Q_{α} represents an optionally substituted hydroxyl group, or an optionally substituted amino group,
(2) W_{α} represents an oxygen atom or a —NT_{α}— group (wherein T_{α} represents a hydrogen atom, or a substituent on the nitrogen atom),
(3) K_{α} and L_{α} form a —V═V_{α}′—V_{α}″═V_{α}′″— group (wherein V_{α}, V_{α}, V_{α}″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (I′-2): 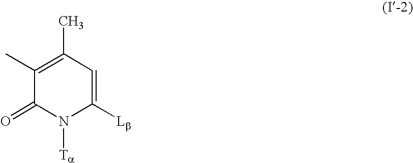
wherein T_{α} is as defined above, and L_{β} represents a hydroxyl group or a methyl group;
a group represented by formula (I′-3): 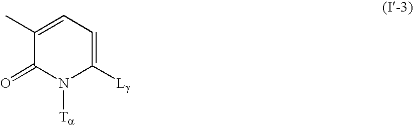
wherein T_{α} is as defined above, and L_{γ} represents a C1-C10 alkyl group;
a group represented by formula (I′-4): 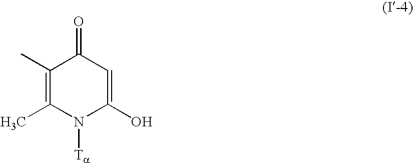
wherein T_{α} is as defined above;
a group represented by formula (I′-5): 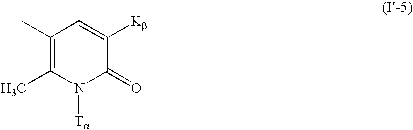
wherein T_{α} is as defined above, and K_{β} represents a cyano group or a UOCO— group (wherein U represents a hydrogen atom or a C1-C10 alkyl group);
a group represented by formula (I′-6): 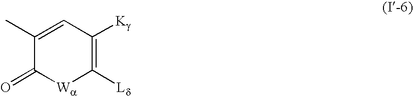
wherein W_{α} is as defined above, and K_{γ} and L_{δ} form an optionally substituted C3-C10 alkylene group or an optionally substituted C4-C10 alkenylene group;
a group represented by formula (I′-7): 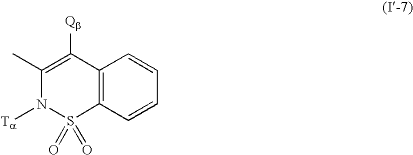
wherein T_{α} is as defined above, and Q_{β} represents an optionally substituted hydroxyl group; or
a group represented by formula (I′-8): 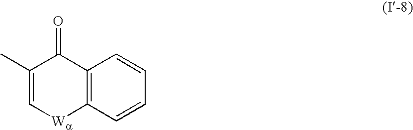
wherein W_{α} is as defined above;
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

13. A cinnamoyl compound represented by formula (II′): 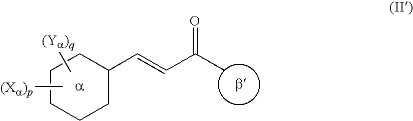
wherein,
I. α represents a benzene ring or a pyridine ring; in (Y_{α})_{q}, Y_{α} is a substituent on a carbon atom and represents a group included in the following X₀ group or the Y₀ group, q represents 0, 1, 2, 3, 4 or 5, and when q is not less than 2, Y_{α}s are the same or different, and when q is not less than 2, adjacent two same or different Y_{α}s may together form a group included in the Z₀ group to be fused to the α ring; and in (X_{α})_{p, X}_{α} is a substituent on a carbon atom and represents a group which does not belong to the following X₀ group, Y₀ group and Z₀ group, p represents 0, 1, 2, 3, 4 or 5, and when p is not less than 2, Xₐ s are the same or different and the sum of p and q is not more than 5;
(1) the X₀ group: a Mₐ-group, wherein Mₐ represents a R_{b}— group (wherein R_{b} represents a C1-C10 alkyl group optionally substituted with a halogen atom), a halogen atom, a nitro group, a cyano group, a hydroxyl group, a R_{c}—Bₐ—R_{d}— group (wherein R_{c} represents a C1-C10 alkyl group optionally substituted with a halogen atom, Bₐ represents an oxy group, a thio group, a sulfinyl group or a sulfonyl group, and R_{d} represents a single bond or a C1-C10 alkylene group), an HOR_{d}— group (wherein R_{d} is as defined above), a Rₑ—CO—R_{d}— group (wherein Rₑ represents a hydrogen atom, or a C1-C10 alkyl group optionally substituted with a halogen atom, and R_{d} is as defined above), a Rₑ—CO—O—R_{d}— group (wherein Rₑ and R_{d} are as defined above), a RₑO—CO—R_{d}— group (wherein Rₑ and R_{d} are as defined above), an HO—CO—CH═CH— group, a RₑRₑ′N—R_{d}— group (wherein Rₑ and Rₑ′ are the same or different, Rₑ is as defined above, Rₑ′ has the same meaning as Rₑ has, and R_{d} is as defined above), a Rₑ—CO—NRₑ′—R_{d}— group (wherein Rₑ, Rₑ′ and R_{d} are as defined above), a R_{b}O—CO—N(Rₑ)—R_{d}— group (wherein R_{b}, Rₑ and R_{d} are as defined above), a RₑRₑ′N—CO—R_{d}— group (wherein Rₑ, Rₑ′ and R_{d} are as defined above), a RₑRₑ′N—CO—NRₑ″—R_{d}— group (wherein Rₑ, Rₑ′ and Rₑ″ are the same or different, Rₑ and Rₑ′ are as defined above, Rₑ″ has the same meaning as Rₑ has, and R_{d} is as defined above), a RₑRₑ′N—C(═NRₑ″)—NRₑ′″—R_{d}— group (wherein Rₑ, Rₑ′, Rₑ″ and Rₑ′″ are the same or different, Rₑ, Rₑ′ and Rₑ″ are as defined above, Rₑ′″ has the same meaning as Rₑ has, and R_{d} is as defined above), a R_{b}—SO₂—NRₑ—R_{d}— group (wherein R_{b}, Rₑ and R_{d} are as defined above), a RₑRₑ′N—SO₂—R_{d}— group (wherein Rₑ, Rₑ′ and R_{d} are as defined above), a C2-C10 alkenyl group or a C2-C10 alkynyl group;
(2) the Y₀ group: a M_{b0}-R_{d}— group, wherein M_{b0} represents a M_{c0}- group
[wherein M_{c0} represents a M_{do}-R_{d}′— group {wherein M_{do} represents a 6 to 10-membered aryl group optionally substituted with a Mₐ- group (wherein Mₐ is as defined above), a 5 to 10-membered heteroaryl group optionally substituted with a Mₐ-group (wherein Mₐ is as defined above), a 3 to 10-membered cyclic hydrocarbon or heterocyclic group optionally substituted with a Mₐ- group (wherein Mₐ is as defined above) and optionally containing an unsaturated bond, a (b₀)- group represented by 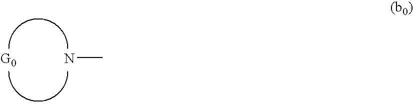
(wherein G₀ forms an optionally substituted, saturated or unsaturated, nonaromatic 5 to 14-membered hydrocarbon ring or heterocyclic ring), a (c₀)- group represented by 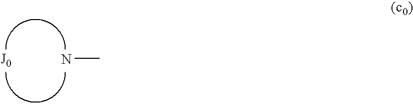
(wherein, J₀ forms a 5 to 7-membered aromatic ring optionally containing a nitrogen atom), a (d₀)- group represented by 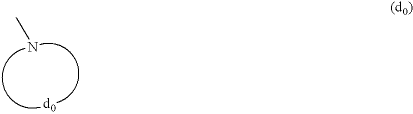
[wherein d₀ forms a 5 to 12-membered hydrocarbon ring which is substituted with a carbonyl group or a thiocarbonyl group and further which may be optionally substituted with an oxy group, a thio group, a —NR₁— group {wherein R₁ represents a hydrogen atom, or a C1-C10 alkyl group, or a C2-C10 alkyl group substituted with a halogen atom or a R₂—B₁— group (wherein R₂ represents a C1-C10 alkyl group, a C3-C 10 alkenyl group or a C3-C10 alkynyl group, and B₁ represents an oxy group, a thio group, a sulfinyl group or a sulfonyl group), a C3-C10 alkenyl group, or a C3-C10 alkynyl group}, a sulfinyl group, or a sulfonyl group], or a (e₀)- group represented by 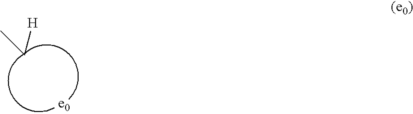
{wherein e₀ forms a 5 to 12-membered hydrocarbon ring optionally substituted with a carbonyl group, a thiocarbonyl group, an oxy group, a thio group, a —NR₁— group (wherein R₁ is as defined above), a sulfinyl group or a sulfonyl group}; and R_{d}′ is the same as or different from R_{d} and has the same meaning as R_{d} has], a M_{c0}-Bₐ— group (wherein M_{c0} and Bₐ are as defined above), a M_{c0}-CO— group (wherein M_{c0} is as defined above), a M_{c0}-CO—O— group (wherein M_{c0} is as defined above), a M_{c0}O—CO— group (wherein M_{c0} is as defined above), a M_{c0}RₑN— group (wherein M_{c0} and Rₑ are as defined above), a M_{c0}-CO—NRₑ— group (wherein M_{c0} and Rₑ are as defined above), a M_{c0}O—CO—NRₑ— group (wherein M_{c0} and Rₑ are as defined above), a M_{c0}RₑN—CO— group (wherein M_{c0} and Rₑ are as defined above), a M_{c0}RₑN—CO—NRₑ′— group (wherein M_{c0}, Rₑ and Rₑ′ are as defined above), a M_{c0}RₑN—C(═NRₑ′)—NRₑ″— group (wherein M_{c0}, Rₑ, Rₑ′ and Rₑ″ are as defined above), a M_{c0}-SO₂—NRₑ— group (wherein M_{c0} and Rₑ are as defined above) or a M_{c0}RₑN—SO₂— group (wherein M_{c0} and Rₑ are as defined above), and R_{d} is as defined above;
(3) the Z₀ group: a 5- to 12-membered hydrocarbon ring or heterocyclic ring which may be substituted with a halogen atom, a C1-C10 alkoxy group, C3-C10 alkenyloxy group, a C3-C10 alkynyloxy group, a carbonyl group, a thiocarbonyl group, an oxy group, a thio group, a sulfinyl group or a sulfonyl group, and which is aromatic or non-aromatic and monocyclic or fused ring and is fused to the α ring; and
II. β′ represents
a group represented by formula (II′-1): 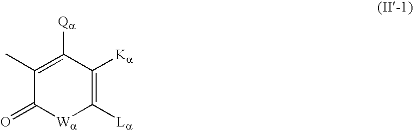
wherein,
(1) Q_{α} represents an optionally substituted hydroxyl group, or an optionally substituted amino group,
(2) W_{α} represents an oxygen atom or a —NTₐ- group (wherein T_{α} represents a hydrogen atom, or a substituent on the nitrogen atom),
(3) K_{α} and L_{α} form a —V_{α}═V_{α}′—V_{α}″═V_{α}′″— group (wherein V_{α}, V_{α}′, Vₐ″ and V_{α}′″ are the same or different, and represent an optionally substituted methine group or a —N═ group, and at least one of V_{α}, V_{α}′, V_{α}″ and V_{α}′″ represents a —N═ group);
a group represented by formula (II′-2): 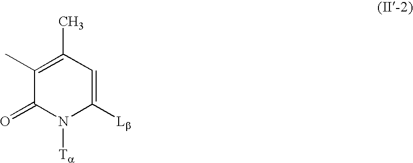
wherein T_{α} is as defined above, and L_{β} represents a hydroxyl group or a methyl group;
a group represented by formula (II′-3): 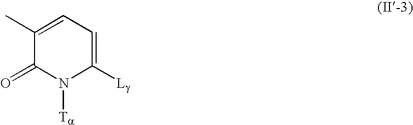
wherein T_{α} is as defined above, and L_{γ} represents a C1-C10 alkyl group;
a group represented by formula (II′-4): 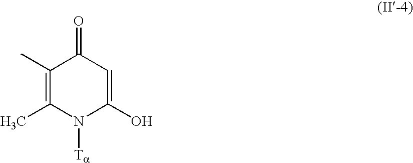
wherein T_{α} is as defined above;
a group represented by formula (II′-5): 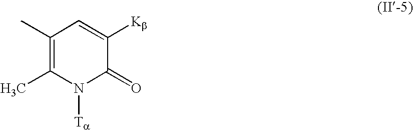
wherein T_{α} is as defined above, and K_{β} represents a cyano group or a UOCO— group (wherein U represents a hydrogen atom or a C1-C10 alkyl group);
a group represented by formula (II′-6): 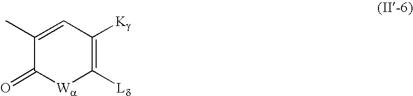
wherein W_{α} is as defined above, and K_{γ} and L_{δ} form an optionally substituted C3-C10 alkylene group or an optionally substituted C4-C10 alkenylene group;
a group represented by formula (II′-7): 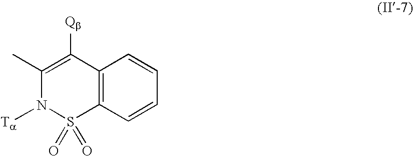
wherein T_{α} is as defined above, and Q_{β} represents an optionally substituted hydroxyl group; or
a group represented by formula (II′-8): 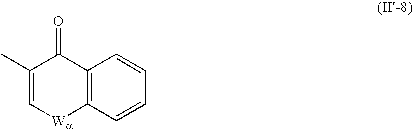
wherein W_{α} is as defined above;
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

14. A cinnamoyl compound represented by formula (V′): 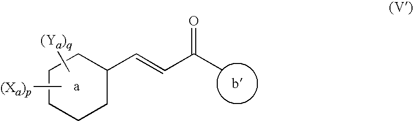
[wherein,
I. a represents a benzene ring or a pyridine ring, Xₐ is a substituent on a carbon atom and represents a C1-C10 alkyl group substituted with a cyano group, or a C1-C10 alkyl group substituted with a tetrahydropyran-4-ylidene group, or a C2-C10 alkenyl group substituted with a halogen atom or a cyano group, or a C2-C10 alkenyl group substituted with a C1-C10 alkoxycarbonyl group, or a C3-C10 alkynyl group substituted with a hydroxyl group, or an a₀-r₁-b-r₁′- group {wherein a₀ represents a methyl group substituted with a C1-C10 alkylthio group, a methyl group substituted with a C1-C10 alkylsulfinyl group, a methyl group substituted with a C1-C10 alkylsulfonyl group, a C2-C10 alkenyl group, a C2-C10 alkynyl group, a r₂O—CO— group (wherein r₂ represents a C1-C10 alkyl group or a C2-C10 alkyl group substituted with a hydroxyl group), a carboxy group, a rr′N—CO— group (wherein r and r′ are the same or different, and represent a hydrogen atom or a C1-C10 alkyl group), a₁-NH—CO— group (wherein a₁ represents a C2-C10 alkyl group substituted with a C1-C10 alkoxy group), an a₁′-CO— group (wherein a₁′ represents a morpholino group), a rr′N—CH₂— group (wherein r and r′ are as defined above), a r₀-(O)₁—CONH—CH₂— group (wherein r₀ represents a C1-C10 alkyl group, and l represents 0 or 1), a r-OCH₂— group (wherein r is as defined above), a r₀-CO— group (wherein r₀ is as defined above), a cyano group or a sulfomethyl group, r₁ represents a C1-C10 alkylene group, r₁′ represents a single bond or a C1-C10 alkylene group, and b represents an oxy group, a thio group, a sulfinyl group, a sulfonyl group, or an imino group}, or an a₂-y-CO—NH— group (wherein a₂ represents a C2-C10 alkyl group substituted with a C1-C10 alkoxy group, and y represents an oxy group or an imino group), or a rO—COCO—NH— group (wherein r is as defined above), or an a₃-z-NH— group (wherein a₃ represents a C2-C10 alkenyl group, or a C1-C10 alkyl group substituted with a C1-C10 alkoxy group, a C1-C10 alkoxycarbonyl group, a carboxy group or a cyano group, and z represents a carbonyl group or a sulfonyl group), or an a₄-NHCO— group {wherein a₄ represents a C1-C10 alkoxy group, or a C3-C10 alkenyloxy group, or a r₀-SO₂— group (wherein r₀ is as defined above), or a C2-C10 alkyl group substituted with a hydroxyl group or a C1-C10 alkoxy group, or a C2-C10 alkyl group substituted with a r₀r₀′N— group (wherein r₀ is as defined above, and r₀′ is the same as or different from r₀, and represents the same meaning as r₀ has), a C1-C10 alkyl group substituted with or a rO—CO— group (wherein r is as defined above), a cyano group or an aminocarbonyl group, or a rO—CO-(rO—COCH₂)CH— group (wherein r is as defined above)}, or an a₅-NHSO₂— group (wherein a₅ represents a C2-C10 alkyl group substituted with a C1-C10 alkoxyl group), or a r₀ON═CH— group (wherein r₀ is as defined above), or a r₀NHCSNH— group (wherein r₀ is as defined above), or a r₀NHC(—Sr₀′)═N— group (wherein r₀ and r₀′ are as defined above), or a (rO)₂P(═O)CH₂— group (wherein r is as defined above), p represents 0, 1, 2 or 3 and, and when p is not less than 2, Xₐs are same or different,
Yₐ represents are halogen atom, or a C1-C10 alkyl group optionally substituted with a halogen atom, or a C1-C10 alkyl group optionally substituted with a C1-C10 alkoxy group, or a C2-C10 alkenyl group, or a C2-C10 alkynyl group, or a 2-oxo-oxazolidin-3-yl group, or a [1,3]dioxolan-2-yl group, or a C1-C10 alkoxy group substituted with a morpholino group, or an a₀′-b′- group (wherein a₀′ represents a C1-C10 alkyl group optionally substituted with a halogen atom, and b′ represents an oxy group, a thio group, a sulfinyl group or a sulfonyl group), or a nitro group, or a cyano group, or a rO—CO— group (wherein r is as defined above), or a r₀r₀′N— group (wherein r₀ and r₀′ are as defined above), or a r₀CO—NH— group (wherein r₀ is as defined above), or a r₀r₀′NCONH— group (wherein r₀ and r₀′ are as defined above), or a rr′NCO— group (wherein r and r′ are as defined above), or a hydroxyl group, q represents 0, 1, 2 or 3, and when q is not less than 2, Yₐs are the same or different, and when q is not less than 2, adjacent Yₐs may be fused to the α ring to form a 4,5,6,7-tetrahydrobenzo[b]thiophene ring;
II. b is
a group represented by formula (V′-1): 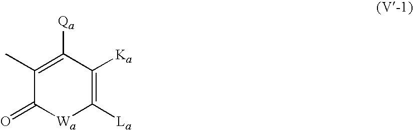
wherein Qₐ represents a rₐ-O— group {rₐ represents a hydrogen atom, or a C1-C10 alkyl group, or a C3-C10 alkenyl group, or a C3-C10 alkynyl, or a C1-C10 alkyl group substituted with a r₀r₀′N—CH₂— group (wherein r₀ and r₀′ are the same or different), a rOCH₂ group (wherein r is as defined above), a r₀-CO— group (wherein r₀ is as defined above), a C1-C10 alkoxycarbonyl group, a carboxy group, an aminocarbonyl group or a cyano group, or a r₃-r₁- group (wherein r₃ represents a phenyl group or a pyridyl group, and r₁ is as defined above)}, or a piperidino group, or a morpholino group, or a r₄r₄′N— group (wherein r₄ and r₄′ are the same of different, and represent a hydrogen atom, or a C1-C10 alkyl group, or C3-C10 alkenyl group, or a C3-C10 alkynyl group, or a C2-C10 alkyl group substituted with a C1-C10 alkoxy group, provided that they are not a hydrogen atom at the same time); W_{α} represents an oxygen atom or a —NTₐ- group [wherein T_{α} represents a r_{b}- group (wherein r_{b} is the same as or different from rₐ, and represents the same meaning as rₐ has) or a r₃′— group (wherein r₃′ is the same as or different from r₃, and represents the same meaning as r₃ has)], and Kₐ and Lₐ form a —Vₐ═Vₐ′—Vₐ″═Vₐ′″— group (wherein Vₐ, Vₐ′, Vₐ″ and Vₐ′″ are the same or different, and represent a methine group or a —N═ group, and at least one of Vₐ, Vₐ′Vₐ″ and Vₐ′″ represents a —N═ group);
a group represented by formula (V′-2): 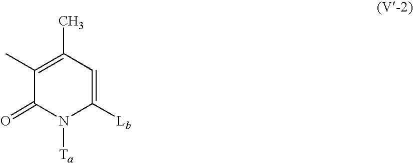
wherein Tₐ is as defined above, and L_{b} represents a hydroxyl group or a methyl group;
a group represented by formula (V′-3): 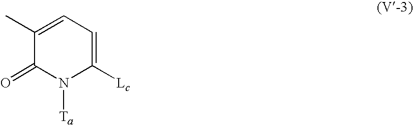
wherein Tₐ is as defined above, and L_{c} represents a C1-C10 alkyl group;
a group represented by formula (V′-4): 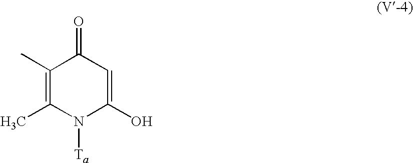
wherein Tₐ is as defined above;
a group represented by formula (V′-5): 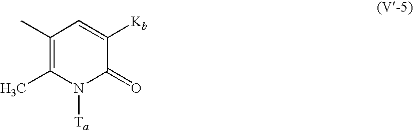
wherein Tₐ is as defined above, and K_{b} represents a cyano group or a UOCO— group (wherein U represents a hydrogen atom or C1-C10 alkyl group);
a group represented by formula (V′-6): 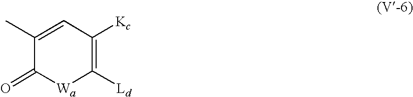
wherein Wₐ is as defined above, and K_{c} and L_{d} form a C3-C10 alkylene group or a C4-C10 alkenylene group;
a group represented by formula (V′-7): 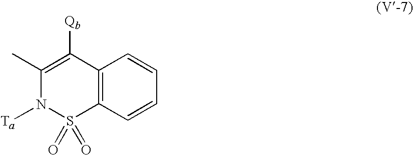
wherein Tₐ is as defined above, and Q_{b} represents a rₐ-O— group (wherein rₐ is as defined above); or
a group represented by formula (V′-8): 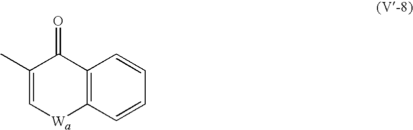
wherein Wₐ is as defined above; and
the term “as defined above” used for the same symbols among plural substituents means that the plural substituents independently represent the same meaning as that described above and, among the plural substituents, although the selection range of substituents to be selected is the same, selected substituents may be the same or different as long as they are selected within the range.

15. A composition for suppressing transcription of an extracellular matrix gene, which comprises the compound according to claim 2, and an inert carrier.

16. A composition for suppressing transcription of an extracellular matrix gene, which comprises the compound according to claim 5, and an inert carrier.

17. Use of a cinnamoyl compound contained in the composition according to claim 1 as an active ingredient, as an active ingredient for suppressing transcription of an extracellular matrix gene.

18. Use of the compound according to claim 2, as an active ingredient for suppressing transcription of an extracellular matrix gene.

19. Use of the compound according to claim 5, as an active ingredient for suppressing transcription of an extracellular matrix gene.

20. Use of a cinnamoyl compound contained in the composition according to claim 1 as an active ingredient, as an active ingredient for decreasing expression of an extracellular matrix gene to induce a reduction in accumulation of an extracellular matrix and thereby improving tissue fibrosis.

21. Use of the compound according to claim 2, as an active ingredient for decreasing expression of an extracellular matrix gene to induce a reduction in accumulation of an extracellular matrix and thereby improving tissue fibrosis.

22. A method for improving tissue fibrosis, which comprises administering an effective amount of a cinnamoyl compound contained in the composition according to claim 1 as an active ingredient to a mammalian patient in need thereof.

23. A method for improving tissue fibrosis, which comprises administering an effective amount of the compound according to claim 2 to a mammalian patient in need thereof.

24. An agent for treating chronic renal failure, which comprises the compound according to claim 2 and an inert carrier.

25. Use of a cinnamoyl compound contained in the composition according to claim 1 as an active ingredient for treating chronic renal failure.

26. A method for treating chronic renal failure, which comprises administering an effective amount of a cinnamoyl compound contained in the composition according to claim 1 as an active ingredient to a mammalian patient in need thereof.

27. An agent for treating heart failure, which comprises a cinnamoyl compound contained in the composition according to claim 1 as an active ingredient, and an inert carrier.

28. Use of a cinnamoyl compound contained in the composition according to claim 1 as an active ingredient for treating heart failure.

29. A method for treating heart failure, which comprises administering an effective amount of a cinnamoyl compound contained in the composition according to claim 1 as an active ingredient to a mammalian patient in need thereof.

30. A composition for suppressing the activity of TGF-β, which comprises a cinnamoyl compound contained in the composition according to claim 1 as an active ingredient, and an inert carrier.

31. A composition for suppressing the activity of TGF-β, which comprises the compound according to claim 2, and an inert carrier.

32. Use of a cinnamoyl compound contained in the composition according to claim 1 as an active ingredient, as an active ingredient for suppressing the activity of TGF-β.

33. Use of the compound according to claim 2, as an active ingredient for suppressing the activity of TGF-β.

34. A composition for hair growth which comprises a cinnamoyl compound contained in the composition according to claim 1 as an active ingredient, and an inert carrier.

35. Use of a cinnamoyl compound contained in the composition according to claim 1 as an active ingredient for inhibiting a promoting effect of TGF-β on transition to a hair regression phase to induce extension of a hair growth phase and thereby providing a hair-growing effect.

36. A method for growing hair, which comprises administering an effective amount of a cinnamoyl compound contained in the composition according to claim 1 as an active ingredient to a mammalian patient in need thereof.


SEQUENCE LISTING (5 sequences)

SEQ ID NO: 1 - DNA, 32 bp, Artificial Sequence
  Synthetic oligonucleotide
ccaagctagcgaaattatcttttctttcatag

SEQ ID NO: 2 - DNA, 28 bp, Artificial Sequence
  Synthetic oligonucleotide
ccaaaagcttgcagtcgtggccagtacc

SEQ ID NO: 3 - DNA, 22 bp, Artificial Sequence
  Synthetic oligonucloetide
tcgccatcagtagaaggtagca

SEQ ID NO: 4 - DNA, 25 bp, Artificial Sequence
  Synthetic oligonucleotide
tatactgaacaccaggttgcaagtc

SEQ ID NO: 5 - DNA, 30 bp, Artificial Sequence
  Synthetic oligonucleotide
ctcaaccttcctgaaactgcaaactccgtc